(19) United States
(12) Patent Application Publication
(10) Pub. No.: US 2004/0235845 A1
(43) Pub. Date: Nov. 25, 2004

(54) Use of phosphodiesterase iv inhibitors

(76) Inventors: Hans-Michael Eggenweiler, Darmstadt (DE); Michael Wolf, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/492,367

(22) PCT Filed: Sep. 19, 2002
(86) PCT No.: PCT/EP02/10507

(30) Foreign Application Priority Data
Oct. 12, 2001 (DE) 101-50-517.5

(51) Int. Cl. A61K 31/501; A61K 31/50

(52) U.S. Cl. 514/247; 514/252.01

(57) ABSTRACT

Use of phosphodiesterase IV inhibitors and/or physiologically acceptable salts thereof for the preparation of a medicament for the treatment of osteoporosis, tumours, tumour metastases, atherosclerosis, rheumatoid arthritis, multiple sclerosis, diabetes mellitus, ulcerative collitis and AIDS.

The invention relates to the use of

a) compounds of the formula I, disclosed in EP 0 738 715 A2,

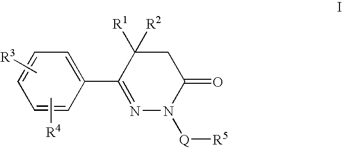

in which

R¹ and R² are each, independently of one another, H or A,

R³ and R⁴ are each, independently of one another, —OH, —OR¹⁰, —S—R¹⁰, —SO—R¹⁰, —SO₂—R¹⁰, Hal, methylenedioxy, —NO₂, —NH₂, —NHR¹⁰ or —NR¹⁰R¹¹,

R⁵ is a phenyl radical which is unsubstituted or monosubstituted or disubstituted by R⁶ and/or R⁷,

Q is absent or is alkylene having 1-6 carbon atoms,

R⁶ and R⁷ are each, independently of one another, —NH₂, —NR⁸R⁹, —NHR¹⁰, —NR¹⁰R¹¹, —NO₂, Hal, —CN, —OA, —COOH or —COOA,

R⁸ and R⁹ are each, independently of one another, H, acyl having 1-8 carbon atoms, which may be substituted by 1-5 F and/or Cl atoms, or —COOA, —SO-A, —SO₂A, —CONH₂, —CONHA, —CONA₂, —CO—COOH, —CO—COOA, —CO—CONH₂, —CO—CONHA or —CO—CONA₂,

A is alkyl having from 1 to 6 carbon atoms, which may be substituted by 1-5 F and/or Cl atoms,

R¹⁰ and R¹¹ are each, independently of one another, A, cycloalkyl having 3-7 carbon atoms, methylenecycloalkyl having 4-8 carbon atoms or alkenyl having 2-8 carbon atoms,

and

Hal is F, Cl, Br or l,

b) compounds of the formula I, disclosed in EP 0 723 962 B1,

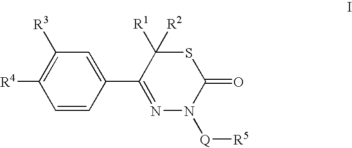

in which

R¹ and R² are each, independently of one another, H or A,

R³ and R⁴ are each, independently of one another, —OH, —OR¹⁰, —S—R¹⁰, —SO—R¹⁰, —SO₂—R¹⁰, Hal, methylenedioxy, —NO₂, —NH₂, —NHR¹⁰ or —NR¹⁰R¹¹,

R⁵ is a phenyl radical which is unsubstituted or monosubstituted or disubstituted by R⁶ and/or R⁷,

Q is absent or is alkylene having 1-6 carbon atoms,

R⁶ and R⁷ are each, independently of one another, —NH₂, —NR⁸R⁹, —NHR¹⁰, —NR¹⁰R¹¹, —NO₂, Hal, —CN, —OA, —COOH or —COOA,

R⁸ and R⁹ are each, independently of one another, H, acyl having 1-8 carbon atoms, which may be substituted by 1-5 F and/or Cl atoms, or —COOA, —SO-A, —SO₂A, —CONH₂, —CONHA, —CONA₂, —CO—COOH, —CO—COOA, —CO—CONH₂, —CO—CONHA or —CO—CONA₂,

A is alkyl having from 1 to 6 carbon atoms, which may be substituted by 1-5 F and/or Cl atoms,

R¹⁰ and R¹¹ are each, independently of one another, A, cycloalkyl having 3-7 carbon atoms, methylenecycloalkyl having 4-8 carbon atoms or alkenyl having 2-8 carbon atoms,

and

Hal is F, Cl, Br or l,

c) compounds of the formula I, disclosed in EP 0 763 534 A1,

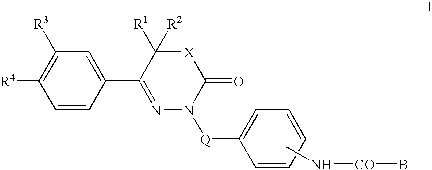

in which

B is an aromatic heterocyclic radical having from 1 to 4 N, O and/or S atoms, bonded via N or C, which may be unsubstituted or monosubstituted, disubstituted or trisubstituted by Hal, A and/or OA, and may also be fused to a benzene or pyridine ring,

Q is absent or is alkylene having 1-6 carbon atoms,

X is CH₂, S or O,

R¹ and R² are each, independently of one another, H or A,

R³ and R⁴ are each, independently of one another, —OH, —OR⁵, —S—R⁵, —SO—R⁵, —SO₂—R⁵, Hal, methylenedioxy, —NO₂, —NH₂, —NHR⁵ or —NR⁵R⁶,

R⁵ and R⁶ are each, independently of one another, A, cycloalkyl having 3-7 carbon atoms, methylenecycloalkyl having 4-8 carbon atoms or alkenyl having 2-8 carbon atoms,

A is alkyl having from 1 to 10 carbon atoms, which may be substituted by from 1 to 5 F and/or Cl atoms, and

Hal is F, Cl, Br or l,

d) compounds of the formula I, disclosed in EP 0 539 806 B1,

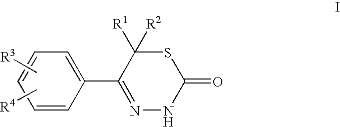

in which

R¹ and R² are each, independently of one another, H or A,

R³ is H, OA or O—CₘH₂ₘ₊₁₋ₙXₙ,

R⁴ is —O—CₘH₂ₘ₊₁₋ₙXₙ,

X is F or Cl,

A is alkyl having 1-6 carbon atoms,

m is 1, 2, 3, 4, 5 or 6, and

n is 1, 2, 3, 4, 5, 6, 7, 8, 9, 10, 11, 12 or 13,

e) compounds of the formula I, disclosed in EP 0 618 201 A1,

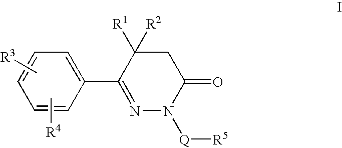

in which

R¹ and R² are each, independently of one another, H or A,

R³ and R⁴ are each, independently of one another, OH, OA, SA, SOA, SO₂A, Hal, methylenedioxy, cycloalkoxy having 3-7 carbon atoms or O—CₘH₂ₘ₊₁₋ₖFₖ,

R⁵ is —NR⁶R⁷ or

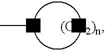

where one CH₂ group may also be replaced by oxygen,

R⁶ and R⁷ are each, independently of one another, H or A,

Q is alkylene having 1-6 carbon atoms,

A is alkyl having 1-6 carbon atoms,

Hal is F, Cl, Br or l,

m is 1, 2, 3, 4, 5 or 6,

n is 3, 4, 5 or 6,

k is 1, 2, 3, 4, 5, 6, 7, 8, 9, 10, 11, 12 or 13,

f) compounds of the formula I, disclosed in DE 196 04 388 A1,

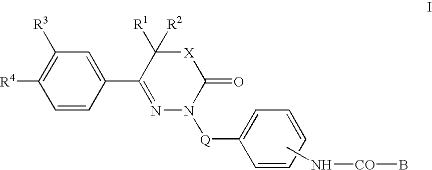

in which

R¹ and R² are each, independently of one another, H or A,

R³ and R⁴ are each, independently of one another, —OH, —OA, —S—A, —SO—A, —SO₂-A, Hal, methylenedioxy, —NO₂, —NH₂, —NHA or —NAA′,

A and A′ are each, independently of one another, alkyl having from 1 to 10 carbon atoms, which may be substituted by from 1 to 5 F and/or Cl atoms, or cycloalkyl having 3-7 carbon atoms or methylenecycloalkyl having 4-8 carbon atoms,

B is —Y—R⁵ or —O—Y—R⁵,

Q is absent or is alkylene having 1-4 carbon atoms,

Y is absent or is alkylene having 1 -10 carbon atoms,

X is CH₂ or S,

R⁵ is NH₂, NHA, NAA′ or a saturated 3-8-membered heterocyclic radical having at least one N atom which is unsubstituted or monosubstituted by A or OH and in which, in addition, further CH₂ groups may be replaced by NH, NA, S or O,

Hal is F, Cl, Br or l,

and/or physiologically acceptable salts thereof for the preparation of a medicament for the treatment of osteoporosis, tumours, tumour metastases, atherosclerosis, rheumatoid arthritis, multiple sclerosis, diabetes mellitus, ulcerative colitis and AIDS.

The invention had the object of finding novel uses of the compounds of the formulae I, in particular those which can lead to the preparation of medicaments.

It has been found that the compounds of the formulae I and their salts have very valuable pharmacological properties and are well tolerated.

In particular, they exhibit selective phosphodiesterase IV inhibition combined with an intracellular increase in cAMP (N. Sommer et al., Nature Medicine, 1, 244-248 (1995)).

The PDE IV inhibition can be demonstrated, for example, analogously to C. W. Davis in Biochim. Biophys. Acta 797, 354-362 (1984).

The compounds according to the invention can be employed for the treatment of asthmatic diseases. The antiasthmatic action of PDE IV inhibitors has been described, for example, by T. J. Torphy et al. in Thorax, 46, 512-523 (1991) and can be determined, for example, by the method of T. Olsson, Acta allergologica 26, 438-447 (1971).

Since cAMP inhibits bone-degrading cells and stimulates bone-forming cells (S. Kasugai et al., M 681 and K. Miyamoto, M 682, in Abstracts of the American Society for Bone and Mineral Research, 18^{th} Annual Meeting, 1996), the compounds according to the invention can be employed for the treatment of osteoporosis.

In addition, the compounds exhibit an inhibiting action to the formation of TNF (tumour necrosis factor) and are therefore suitable for the treatment of allergic and inflammatory illnesses, autoimmune diseases and transplant rejection reactions. They can be employed for the treatment of memory disorders, tumours, tumour metastases, atherosclerosis, rheumatoid arthritis, multiple sclerosis, Crohn's disease, atopic dermatitis, diabetes mellitus, ulcerative colitis and AIDS.

The action of PDE IV inhibitors in the treatment of asthma, inflammatory diseases, diabetes mellitus, atopic dermatitis, psoriasis, AIDS, tumour growth or tumour metastases is described, for example in EP 779 291.

The antiinflammatory action of the substances according to the invention and their efficacy for the treatment of, for example, autoimmune diseases, multiple sclerosis or rheumatoid arthritis, can be determined analogously to the methods of N. Sommer et al., Nature Medicine 1, 244-248 (1995) or L. Sekut et al., Clin. Exp. Immunol. 100, 126-132 (1995).

The action of PDE IV inhibitors in the treatment of tumours is described, for example, in WO 95/35281, WO 95/17399 and WO 96/00215.

Surprisingly, it has been found that the compounds of the formula I are suitable for the treatment of cancer.

The invention furthermore relates to the use of the compounds of the formulae I and their physiologically acceptable salts and/or solvates for the preparation of a medicament for inhibiting the growth of neoplastic cells.

The term neoplastic cells is taken to mean cancer cells.

The invention relates in particular to the use of the compounds of the formulae I and their physiologically acceptable salts and/or solvates for the preparation of a medicament for the treatment and/or prophylaxis of cancer diseases.

The invention furthermore relates to the use of the compounds of the formulae I and their physiologically acceptable salts and/or solvates for the preparation of a medicament for the treatment of neoplastic damage.

The invention furthermore relates to the use of the compounds of the formulae I and their physiologically acceptable salts and/or solvates for the preparation of a medicament for the treatment of pre-cancerogenic damage.

The term precancerogenic damage is taken to mean, for example, benign tumours in the intestine which could lead to intestinal cancer.

The term precancerogenic damage is taken to mean, in particular, the lesions mentioned in U.S. Pat. No. 5,948,911 in column 4, lines 49-60.

Irregularities in apoptosis (cell death) play a part in the formation of precancerogenic damage.

It is also known that the regulation of apoptosis plays an important role in diseases connected with abnormal cell growth, such as, for example, benign prostate hyperplasia, neurodegenerative diseases, such as, for example, Parkinson's, autoimmune diseases, including multiple sclerosis, and rheumatoid arthritis, or infection diseases, such as AIDS.

The compounds of the formulae I modulate apoptosis and are used in the treatment or prophylaxis of cancer diseases.

The invention thus relates to the use of the compounds of the formulae I and their physiologically acceptable salts and/or solvates for the preparation of a medicament for the regulation of apoptosis in human cells.

The compounds of the formulae I can be employed as medicament active ingredients in human and veterinary medicine. They can furthermore be employed as intermediates for the preparation of further medicament active ingredients.

The compounds of the formulae I can have a chiral centre and can therefore occur in a plurality of stereoisomeric forms. All these forms (for example R and S forms) and mixtures thereof (for example the R,S forms) are included in the formulae I.

These compounds of the formulae I can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise assistants, such as lubricants, preservatives, stabilisers and/or wefting agents, emulsifiers, salts for modifying the osmotic pressure, buffer substances, colorants and flavours and/or one or more further active ingredients, for example one or more vitamins.

The compounds of the formulae I and their physiologically acceptable salts can be employed for combating illnesses in which an increase in the cAMP (cycloadenosine monophosphate) level results in inflammation inhibition or prevention and muscle relaxation.

The compounds according to the invention can be used, in particular, in the treatment osteoporosis, tumours, tumour metastases, asthma, chronic bronchitis, atopic dermatitis, psoriasis and skin diseases and autoimmune diseases.

The substances according to the invention are in general preferably administered in doses of between about 1 and 500 mg, in particular between 5 and 100 mg per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

Compounds of the formulae I can have one or more centres of asymmetry. In this case, they are usually in racemic form. Racemates obtained can be separated into their enantiomers mechanically or chemically by methods known per se. Diastereomers are preferably formed from the racemic mixtures by reaction with an optically active resolving agent.

The invention relates, in particular, to the use of

a) compounds, disclosed in EP 0 738 715 A2, selected from the group consisting of

2-(4-nitrobenzyl)-6-(3,4-dimethoxyphenyl)-2,3,4,5-tetrahydropyridazin-3-one, m.p. 126°;

2-(3-nitrobenzyl)-6-(3,4-dimethoxyphenyl)-2,3,4,5-tetrahydropyridazin-3-one, m.p. 122°;

2-(2-nitrobenzyl)-6-(3,4-dimethoxyphenyl)-2,3,4,5-tetrahydropyridazin-3-one;

2-(2,3-dinitrobenzyl)-6-(3,4-dimethoxyphenyl)-2,3,4,5-tetrahydropyridazin-3-one;

2-(2,4-dinitrobenzyl)-6-(3,4-dimethoxyphenyl)-2,3,4,5-tetrahydropyridazin-3-one;

2-(2-methoxybenzyl)-6-(3,4-dimethoxyphenyl)-2,3,4,5-tetrahydropyridazin-3-one;

2-(4-methoxybenzyl)-6-(3,4-dimethoxyphenyl )-2,3,4,5-tetrahydropyridazin-3-one;

2-(2-chlorobenzyl)-6-(3,4-dimethoxyphenyl)-2,3,4,5-tetrahydropyridazin-3-one;

2-(2,6-dichlorobenzyl)-6-(3,4-dimethoxyphenyl)-2,3,4,5-tetrahydropyridazin-3-one;

2-(4-cyanobenzyl)-6-(3,4-dimethoxyphenyl)-2,3,4,5-tetrahydropyridazin-3-one;

2-(4-carboxybenzyl)-6-(3,4-dimethoxyphenyl)-2,3,4,5-tetrahydropyridazin-3-one;

2-(4-nitrobenzyl)-6-(3,4-dimethoxyphenyl)-5-ethyl-2,3,4,5-tetrahydropyridazin-3-one;

2-(3-nitrobenzyl)-6-(3,4-dimethoxyphenyl)-5-ethyl-2,3,4,5-tetrahydropyridazin-3-one;

2-(2-nitrobenzyl)-6-(3,4-dimethoxyphenyl)-5-ethyl-2,3,4,5-tetrahydropyridazin-3-one;

2-(2,3-dinitrobenzyl)-6-(3,4-dimethoxyphenyl)-5-ethyl-2,3,4,5-tetrahydropyridazin-3-one;

2-(2,4-dinitrobenzyl)-6-(3,4-dimethoxyphenyl)-5-ethyl-2,3,4,5-tetrahydropyridazin-3-one;

2-(2-methoxybenzyl)-6-(3,4-dimethoxyphenyl)-5-ethyl-2,3,4,5-tetrahydropyridazin-3-one;

2-(4-methoxybenzyl)-6-(3,4-dimethoxyphenyl)-5-ethyl-2,3,4,5-tetrahydropyridazin-3-one;

2-(2-chlorobenzyl)-6-(3,4-dimethoxyphenyl)-5-ethyl-2,3,4,5-tetrahydropyridazin-3-one;

2-(2,6-dichlorobenzyl)-6-(3,4-dimethoxyphenyl)-5-ethyl-2,3,4,5-tetrahydropyridazin-3-one;

2-(4-cyanobenzyl)-6-(3,4-dimethoxyphenyl)-5-ethyl-2,3,4,5-tetrahydropyridazin-3-one;

2-(4-carboxybenzyl)-6-(3,4-dimethoxyphenyl)-5-ethyl-2,3,4,5-tetrahydropyridazin-3-one;

2-(4-nitrobenzyl)-6-(3-methoxy-4-trifluoromethoxyphenyl)-5-ethyl-2,3,4,5-tetrahydropyridazin-3-one;

2-(4-nitrobenzyl)-6-(3-methoxy-4-difluoromethoxyphenyl)-5-ethyl-2,3,4,5-tetrahydropyridazin-3-one;

2-(4-nitrobenzyl)-6-(3-methoxy-4-fluoromethoxyphenyl)-5-ethyl-2,3,4,5-tetrahydropyridazin-3-one;

2-(4-nitrobenzyl)-6-(3-difluoromethoxy-4-methoxyphenyl)-5-ethyl-2,3,4,5-tetrahydropyridazin-3-one;

2-(4-nitrobenzyl)-6-(3-trifluoromethoxy-4-methoxyphenyl)-5-ethyl-2,3,4,5-tetrahydropyridazin-3-one;

2-(4-nitrobenzyl)-6-(3-fluoromethoxy-4-methoxyphenyl)-5-ethyl-2,3,4,5-tetrahydropyridazin-3-one;

2-(4-nitrobenzyl)-6-(3-methoxy-4-ethoxyphenyl)-5-ethyl-2,3,4,5-tetrahydropyridazin-3-one;

2-(4-nitrobenzyl)-6-(3-ethoxy-4-methoxyphenyl)-5-ethyl-2,3,4,5-tetrahydropyridazin-3-one;

2-(4-nitrobenzyl)-6-(3-hydroxy-4-methoxyphenyl)-5-ethyl-2,3,4,5-tetrahydropyridazin-3-one;

2-(4-nitrobenzyl)-6-(4-methylsulfonylphenyl)-5-ethyl-2,3,4,5-tetrahydropyridazin-3-one;

2-(4-nitrobenzyl)-6-(4-methyleneoxyphenyl)-5-ethyl-2,3,4,5-tetrahydropyridazin-3-one;

2-(4-nitrobenzyl)-6-(3-cyclopentyloxy-4-methoxyphenyl)-5-ethyl-2,3,4,5-tetrahydropyridazin-3-one;

2-(4-aminobenzyl)-6-(3,4-dimethoxyphenyl)-2,3,4,5-tetrahydropyridazin-3-one, m.p. 184°;

2-(3-aminobenzyl)-6-(3,4-dimethoxyphenyl)-2,3,4,5-tetrahydropyridazin-3-one, m.p. 140°;

2-(2-aminobenzyl)-6-(3,4-dimethoxyphenyl)-2,3,4,5-tetrahydropyridazin-3-one;

2-(2,3-diaminobenzyl)-6-(3,4-dimethoxyphenyl)-2,3,4,5-tetrahydropyridazin-3-one;

2-(2,4-diaminobenzyl)-6-(3,4-dimethoxyphenyl)-2,3,4,5-tetrahydropyridazin-3-one;

2-(4-aminobenzyl)-6-(3,4-dimethoxyphenyl)-5-ethyl-2,3,4,5-tetrahydropyridazin-3-one;

2-(3-aminobenzyl)-6-(3,4-dimethoxyphenyl)-5-ethyl-2,3,4,5-tetrahydropyridazin-3-one, m.p. 49°;

2-(2-aminobenzyl)-6-(3,4-dimethoxyphenyl)-5-ethyl-2,3,4,5-tetrahydropyridazin-3-one;

2-(2,3-diaminobenzyl)-6-(3,4-dimethoxyphenyl)-5-ethyl-2,3,4,5-tetrahydropyridazin-3-one;

2-(2,4-diaminobenzyl)-6-(3,4-dimethoxyphenyl)-5-ethyl-2,3,4,5-tetrahydropyridazin-3-one;

2-(4-aminobenzyl)-6-(3-methoxy-4-trifluoromethoxyphenyl)-5-ethyl-2,3,4,5-tetrahydropyridazin-3-one;

2-(4-aminobenzyl)-6-(3-methoxy-4-difluoromethoxyphenyl)-5-ethyl-2,3,4,5-tetrahydropyridazin-3-one;

2-(4-aminobenzyl)-6-(3-methoxy-4-fluoromethoxyphenyl)-5-ethyl-2,3,4,5-tetrahydropyridazin-3-one;

2-(4-aminobenzyl)-6-(3-difluoromethoxy-4-methoxyphenyl)-5-ethyl-2,3,4,5-tetrahydropyridazin-3-one;

2-(4-aminobenzyl)-6-(3-trifluoromethoxy-4-methoxyphenyl)-5-ethyl-2,3,4,5-tetrahydropyridazin-3-one;

2-(4-aminobenzyl)-6-(3-fluoromethoxy-4-methoxyphenyl)-5-ethyl-2,3,4,5-tetrahydropyridazin-3-one;

2-(4-aminobenzyl)-6-(3-methoxy-4-ethoxyphenyl)-5-ethyl-2,3,4,5-tetrahydropyridazin-3-one;

2-(4-aminobenzyl)-6-(3-ethoxy-4-methoxyphenyl )-5-ethyl-2,3,4,5-tetrahydropyridazin-3-one;

2-(4-aminobenzyl)-6-(3-hydroxy-4-methoxyphenyl)-5-ethyl-2,3,4,5-tetrahydropyridazin-3-one;

2-(4-aminobenzyl)-6-(4-methylsulfonylphenyl)-5-ethyl-2,3,4,5-tetrahydropyridazin-3-one;

2-(4-aminobenzyl)-6-(4-methyleneoxyphenyl)-5-ethyl-2,3,4,5-tetrahydropyridazin-3-one;

2-(4-aminobenzyl)-6-(3-cyclopentyloxy-4-methoxyphenyl)-5-ethyl-2,3,4,5-tetrahydropyridazin-3-one;

2-(3-aminobenzyl)-6-(3-cyclopentyloxy-4-methoxyphenyl)-5-ethyl-2,3,4,5-tetrahydropyridazin-3-one, m.p. 109°;

2-(4-aminophenethyl)-6-(3,4-dimethoxyphenyl)-2,3,4,5-tetrahydropyridazin-3-one;

2-(4-aminophenethyl)-6-(3,4-dimethoxyphenyl)-5-ethyl-2,3,4,5-tetrahydropyridazin-3-one;

2-(3-aminobenzyl)-6-(3-ethoxy-4-methoxyphenyl)-2,3,4,5-tetrahydropyridazin-3-one, m.p. 112°;

2-(4-carboxybenzyl)-6-(3,4-dihydroxyphenyl )-5-ethyl-2,3,4,5-tetrahydropyridazin-3-one;

2-(4-butyrylaminobenzyl)-6-(3,4-dimethoxyphenyl)-2,3,4,5-tetrahydropyridazin-3-one, m.p. 148°;

2-(4-acetamidobenzyl)-6-(3,4-dimethoxyphenyl)-2,3,4,5-tetrahydropyridazin-3-one, m.p. 183°;

2-(4-trifluoroacetamidobenzyl)-6-(3,4-dimethoxyphenyl)-2,3,4,5-tetrahydropyridazin-3-one, m.p. 210°;

2-(4-methylsulfonamidobenzyl)-6-(3,4-dimethoxyphenyl)-2,3,4,5-tetrahydropyridazin-3-one, m.p. 138°;

2-(4-propionylaminobenzyl)-6-(3,4-dimethoxyphenyl)-2,3,4,5-tetrahydropyridazin-3-one, m.p. 176°;

2-(4-tert-butylcarbonylaminobenzyl)-6-(3,4-dimethoxyphenyl)-2,3,4,5-tetrahydropyridazin-3-one, m.p. 155°;

2-(4-isobutyrylaminobenzyl)-6-(3,4-dimethoxyphenyl)-2,3,4,5-tetrahydropyridazin-3-one;

2-(4-methoxycarbonylaminobenzyl)-6-(3,4-dimethoxyphenyl)-2,3,4,5-tetrahydropyridazin-3-one;

2-(4-pivalylaminobenzyl)-6-(3,4-dimethoxyphenyl)-2,3,4,5-tetrahydropyridazin-3-one;

2-(4-cyclopentylcarbamoylbenzyl)-6-(3,4-dimethoxyphenyl)-2,3,4,5-tetrahydropyridazin-3-one;

2-(4-ethoxycarbonylaminobenzyl)-6-(3,4-dimethoxyphenyl)-2,3,4,5-tetrahydropyridazin-3-one, m.p. 147°;

2-(4-methoxalylaminobenzyl)-6-(3,4-dimethoxyphenyl)-2,3,4,5-tetrahydropyridazin-3-one;

2-(4-ureidobenzyl)-6-(3,4-dimethoxyphenyl)-2,3,4,5-tetrahydropyridazin-3-one;

2-(4-pentanoylaminobenzyl)-6-(3,4-dimethoxyphenyl)-2,3,4,5-tetrahydropyridazin-3-one;

2-(4-hexanoylaminobenzyl)-6-(3,4-dimethoxyphenyl)-2,3,4,5-tetrahydropyridazin-3-one;

2-(4-pentafluoropropionylaminobenzyl)-6-(3,4-dimethoxyphenyl)-2,3,4,5-tetrahydropyridazin-3-one;

2-(4-acetamidobenzyl)-6-(3,4-dimethoxyphenyl)-5-ethyl-2,3,4,5-tetrahydropyridazin-3-one;

2-(4-trifluoroacetamidobenzyl)-6-(3,4-dimethoxyphenyl)-5-ethyl-2,3,4,5-tetrahydropyridazin-3-one, m.p. 133°;

2-(4-methylsulfonamidobenzyl)-6-(3,4-dimethoxyphenyl)-5-ethyl-2,3,4,5-tetrahydropyridazin-3-one;

2-(4-propionylaminobenzyl)-6-(3,4-dimethoxyphenyl)-5-ethyl-2,3,4,5-tetrahydropyridazin-3-one, m.p. 81°;

2-(4-tert-butylcarbonylaminobenzyl)-6-(3,4-dimethoxyphenyl)-5-ethyl-2,3,4,5-tetrahydropyridazin-3-one;

2-(4-butyrylaminobenzyl)-6-(3,4-dimethoxyphenyl)-5-ethyl-2,3,4,5-tetrahydropyridazin-3-one, m.p. 117°;

2-(4-isobutyrylaminobenzyl)-6-(3,4-dimethoxyphenyl)-5-ethyl-2,3,4,5-tetrahydropyridazin-3-one;

2-(4-methoxycarbonylaminobenzyl)-6-(3,4-dimethoxyphenyl)-5-ethyl-2,3,4,5-tetrahydropyridazin-3-one, m.p. 144°;

2-(4-pivalylaminobenzyl)-6-(3,4-dimethoxyphenyl)-5-ethyl-2,3,4,5-tetrahydropyridazin-3-one;

2-(4-cyclopentylcarbamoylbenzyl)-6-(3,4-dimethoxyphenyl)-5-ethyl-2,3,4,5-tetrahydropyridazin-3-one;

2-(4-ethoxycarbonylaminobenzyl)-6-(3,4-dimethoxyphenyl)-5-ethyl-2,3,4,5-tetrahydropyridazin-3-one, m.p. 154°;

2-(4-methoxalylaminobenzyl)-6-(3,4-dimethoxyphenyl)-5-ethyl-2,3,4,5-tetrahydropyridazin-3-one;

b 2-(4-ureidobenzyl)-6-(3,4-dimethoxyphenyl)-5-ethyl-2,3,4,5-tetrahydropyridazin-3-one;

2-(4-pentanoylaminobenzyl)-6-(3,4-dimethoxyphenyl)-5-ethyl-2,3,4,5-tetrahydropyridazin-3-one;

2-(4-hexanoylaminobenzyl)-6-(3,4-dimethoxyphenyl)-5-ethyl-2,3,4,5-tetrahydropyridazin-3-one;

2-(4-pentafluoropropionylaminobenzyl)-6-(3,4-dimethoxyphenyl)-5-ethyl-2,3,4,5-tetrahydropyridazin-3-one;

2-(4-acetamidobenzyl)-6-(3-ethoxy-4-methoxyphenyl)-5-ethyl-2,3,4,5-tetrahydropyridazin-3-one;

2-(4-trifluoroacetamidobenzyl)-6-(3-ethoxy-4-methoxyphenyl)-5-ethyl-2,3,4,5-tetrahydropyridazin-3-one;

2-(4-methylsulfonamidobenzyl)-6-(3-ethoxy-4-methoxyphenyl)-5-ethyl-2,3,4,5-tetrahydropyridazin-3-one;

2-(4-propionylaminobenzyl)-6-(3-ethoxy-4-methoxyphenyl)-5-ethyl-2,3,4,5-tetrahydropyridazin-3-one;

2-(4-butyrylaminobenzyl)-6-(3-ethoxy-4-methoxyphenyl)-5-ethyl-2,3,4,5-tetrahydropyridazin-3-one;

2-(4-isobutyrylaminobenzyl)-6-(3-ethoxy-4-methoxyphenyl)-5-ethyl-2,3,4,5-tetrahydropyridazin-3-one;

2-(4-methoxycarbonylaminobenzyl)-6-(3-ethoxy-4-methoxyphenyl)-5-ethyl-2,3,4,5-tetrahydropyridazin-3-one;

2-(4-pivalylaminobenzyl)-6-(3-ethoxy-4-methoxyphenyl)-5-ethyl-2,3,4,5-tetrahydropyridazin-3-one;

2-(4-cyclopentylcarbamoylbenzyl)-6-(3-ethoxy-4-methoxyphenyl)-5-ethyl-2,3,4,5-tetrahydropyridazin-3-one;

2-(4-ethoxycarbonylaminobenzyl)-6-(3-ethoxy-4-methoxyphenyl)-5-ethyl-2,3,4,5-tetrahydropyridazin-3-one;

2-(4-methoxalylaminobenzyl)-6-(3-ethoxy-4-methoxyphenyl)-5-ethyl-2,3,4,5-tetrahydropyridazin-3-one;

2-(4-ureidobenzyl)-6-(3-ethoxy-4-methoxyphenyl)-5-ethyl-2,3,4,5-tetrahydropyridazin-3-one;

2-(4-pentanoylaminobenzyl)-6-(3-ethoxy-4-methoxyphenyl)-5-ethyl-2,3,4,5-tetrahydropyridazin-3-one;

2-(4-hexanoylaminobenzyl)-6-(3-ethoxy-4-methoxyphenyl)-5-ethyl-2,3,4,5-tetrahydropyridazin-3-one;

2-(4-pentafluoropropionylaminobenzyl)-6-(3-ethoxy-4-methoxyphenyl)-5-ethyl-2,3,4,5-tetrahydropyridazin-3-one;

2-(4-acetamidobenzyl)-6-(3-cyclopentyloxy-4-methoxyphenyl)-5-ethyl-2,3,4,5-tetrahydropyridazin-3-one;

2-(4-trifluoroacetamidobenzyl)-6-(3-cyclopentyloxy-4-methoxyphenyl )-5-ethyl-2,3,4,5-tetrahydropyridazin-3-one, m.p. 162°;

2-(4-methylsulfonamidobenzyl)-6-(3-cyclopentyloxy-4-methoxyphenyl)-5-ethyl-2,3,4,5-tetrahydropyridazin-3-one;

2-(4-propionylaminobenzyl)-6-(3-cyclopentyloxy-4-methoxyphenyl)-5-ethyl-2,3,4,5-tetrahydropyridazin-3-one, m.p. 69°;

2-(4-tert-butylcarbonylaminobenzyl)-6-(3-cyclopentyloxy-4-methoxyphenyl)-5-ethyl-2,3,4,5-tetrahydropyridazin-3-one;

2-(4-butyrylaminobenzyl)-6-(3-cyclopentyloxy-4-methoxyphenyl)-5-ethyl-2,3,4,5-tetrahydropyridazin-3-one;

2-(4-isobutyrylaminobenzyl)-6-(3-cyclopentyloxy-4-methoxyphenyl)-5-ethyl-2,3,4,5-tetrahydropyridazin-3-one;

2-(4-methoxycarbonylaminobenzyl)-6-(3-cyclopentyloxy-4-methoxyphenyl)-5-ethyl-2,3,4,5-tetrahydropyridazin-3-one;

2-(4-pivalylaminobenzyl)-6-(3-cyclopentyloxy-4-methoxyphenyl)-5-ethyl-2,3,4,5-tetrahydropyridazin-3-one;

2-(4-cyclopentylcarbamoylbenzyl)-6-(3-cyclopentyloxy-4-methoxyphenyl)-5-ethyl-2,3,4,5-tetrahydropyridazin-3-one;

2-(4-ethoxycarbonylaminobenzyl)-6-(3-cyclopentyloxy-4-methoxyphenyl)-5-ethyl-2,3,4,5-tetrahydropyridazin-3-one, m.p. 73°;

2-(4-methoxalylaminobenzyl)-6-(3-cyclopentyloxy-4-methoxyphenyl )-5-ethyl-2,3,4,5-tetrahydropyridazin-3-one;

2-(4-ureidobenzyl)-6-(3-cyclopentyloxy-4-methoxyphenyl)-5-ethyl-2,3,4,5-tetrahydropyridazin-3-one;

2-(4-pentanoylaminobenzyl)-6-(3-cyclopentyloxy-4-methoxyphenyl)-5-ethyl-2,3,4,5-tetrahydropyridazin-3-one;

2-(4-hexanoylaminobenzyl)-6-(3-cyclopentyloxy-4-methoxyphenyl)-5-ethyl-2,3,4,5-tetrahydropyridazin-3-one;

2-(4-pentafluoropropionylaminobenzyl)-6-(3-cyclopentyloxy-4-methoxyphenyl)-5-ethyl-2,3,4,5-tetrahydropyridazin-3-one;

2-(4-acetamidophenethyl)-6-(3,4-dimethoxyphenyl)-5-ethyl-2,3,4,5-tetrahydropyridazin-3-one;

2-(4-trifluoroacetamidophenethyl)-6-(3,4-dimethoxyphenyl)-5-ethyl-2,3,4,5-tetrahydropyridazin-3-one;

2-(4-methylsulfonamidophenethyl)-6-(3,4-dimethoxyphenyl)-5-ethyl-2,3,4,5-tetrahydropyridazin-3-one;

2-(4-propionylaminophenethyl)-6-(3,4-dimethoxyphenyl)-5-ethyl-2,3,4,5-tetrahydropyridazin-3-one;

2-(4-butyrylaminophenethyl)-6-(3,4-dimethoxyphenyl)-5-ethyl-2,3,4,5-tetrahydropyridazin-3-one;

2-(4-isobutyrylaminophenethyl)-6-(3,4-dimethoxyphenyl)-5-ethyl-2,3,4,5-tetrahydropyridazin-3-one;

2-(4-methoxycarbonylaminophenethyl)-6-(3,4-dimethoxyphenyl)-5-ethyl-2,3,4,5-tetrahydropyridazin-3-one;

2-(4-pivalylaminophenethyl)-6-(3,4-dimethoxyphenyl)-5-ethyl-2,3,4,5-tetrahydropyridazin-3-one;

2-(4-cyclopentylcarbamoylphenethyl)-6-(3,4-dimethoxyphenyl)-5-ethyl-2,3,4,5-tetrahydropyridazin-3-one;

2-(4-ethoxycarbonylaminophenethyl)-6-(3,4-dimethoxyphenyl)-5-ethyl-2,3,4,5-tetrahydropyridazin-3-one;

2-(4-methoxalylaminophenethyl)-6-(3,4-dimethoxyphenyl)-5-ethyl-2,3,4,5-tetrahydropyridazin-3-one;

2-(4-ureidophenethyl)-6-(3,4-dimethoxyphenyl)-5-ethyl-2,3,4,5-tetrahydropyridazin-3-one;

2-(4-pentanoylaminophenethyl)-6-(3,4-dimethoxyphenyl)-5-ethyl-2,3,4,5-tetrahydropyridazin-3-one;

2-(4-hexanoylaminophenethyl)-6-(3,4-dimethoxyphenyl)-5-ethyl-2,3,4,5-tetrahydropyridazin-3-one;

2-(4-pentafluoropropionylaminophenethyl)-6-(3,4-dimethoxyphenyl)-5-ethyl-2,3,4,5-tetrahydropyridazin-3-one;

2-(3-acetamidobenzyl)-6-(3,4-dimethoxyphenyl)-5-ethyl-2,3,4,5-tetrahydropyridazin-3-one;

2-(3-trifluoroacetamidobenzyl)-6-(3,4-dimethoxyphenyl )-5-ethyl-2,3,4,5-tetrahydropyridazin-3-one, m.p. 142°;

2-(3-methylsulfonamidobenzyl)-6-(3,4-dimethoxyphenyl)-5-ethyl-2,3,4,5-tetrahydropyridazin-3-one;

2-(3-propionylaminobenzyl)-6-(3,4-dimethoxyphenyl)-5-ethyl-2,3,4,5-tetrahydropyridazin-3-one, m.p. 126°;

2-(3-butyrylaminobenzyl)-6-(3,4-dimethoxyphenyl)-5-ethyl-2,3,4,5-tetrahydropyridazin-3-one;

2-(3-isobutyrylaminobenzyl)-6-(3,4-dimethoxyphenyl)-5-ethyl-2,3,4,5-tetrahydropyridazin-3-one;

2-(3-methoxycarbonylaminobenzyl)-6-(3,4-dimethoxyphenyl)-5-ethyl-2,3,4,5-tetrahydropyridazin-3-one;

2-(3-pivalylaminobenzyl)-6-(3,4-dimethoxyphenyl)-5-ethyl-2,3,4,5-tetrahydropyridazin-3-one;

2-(3-cyclopentylcarbamoylbenzyl)-6-(3,4-dimethoxyphenyl)-5-ethyl-2,3,4,5-tetrahydropyridazin-3-one;

2-(3-ethoxycarbonylaminobenzyl)-6-(3,4-dimethoxyphenyl)-5-ethyl-2,3,4,5-tetrahydropyridazin-3-one, m.p. 54°;

2-(3-methoxalylaminobenzyl)-6-(3,4-dimethoxyphenyl)-5-ethyl-2,3,4,5-tetrahydropyridazin-3-one;

2-(3-ureidobenzyl)-6-(3,4-dimethoxyphenyl)-5-ethyl-2,3,4,5-tetrahydropyridazin-3-one;

2-(3-pentanoylaminobenzyl)-6-(3,4-dimethoxyphenyl)-5-ethyl-2,3,4,5-tetrahydropyridazin-3-one;

2-(3-hexanoylaminobenzyl)-6-(3,4-dimethoxyphenyl)-5-ethyl-2,3,4,5-tetrahydropyridazin-3-one;

2-(3-pentafluoropropionylaminobenzyl)-6-(3,4-dimethoxyphenyl)-5-ethyl-2,3,4,5-tetrahydropyridazin-3-one;

2-(4-acetamidobenzyl)-6-(3-fluoromethoxy-4-methoxyphenyl)-5-ethyl-2,3,4,5-tetrahydropyridazin-3-one;

2-(4-trifluoroacetamidobenzyl)-6-(3-fluoromethoxy-4-methoxyphenyl)-5-ethyl-2,3,4,5-tetrahydropyridazin-3-one;

2-(4-methylsulfonamidobenzyl)-6-(3-fluoromethoxy-4-methoxyphenyl)-5-ethyl-2,3,4,5-tetrahydropyridazin-3-one;

2-(4-propionylaminobenzyl)-6-(3-fluoromethoxy-4-methoxyphenyl)-5-ethyl-2,3,4,5-tetrahydropyridazin-3-one;

2-(4-butyrylaminobenzyl)-6-(3-fluoromethoxy-4-methoxyphenyl)-5-ethyl-2,3,4,5-tetrahydropyridazin-3-one;

2-(4-isobutyrylaminobenzyl)-6-(3-fluoromethoxy-4-methoxyphenyl)-5-ethyl-2,3,4,5-tetrahydropyridazin-3-one;

2-(4-methoxycarbonylaminobenzyl)-6-(3-fluoromethoxy-4-methoxyphenyl)-5-ethyl-2,3,4,5-tetrahydropyridazin-3-one;

2-(4-pivalylaminobenzyl)-6-(3-fluoromethoxy-4-methoxyphenyl)-5-ethyl-2,3,4,5-tetrahydropyridazin-3-one;

2-(4-cyclopentylcarbamoylbenzyl)-6-(3-fluoromethoxy-4-methoxyphenyl)-5-ethyl-2,3,4,5-tetrahydropyridazin-3-one;

2-(4-ethoxycarbonylaminobenzyl)-6-(3-fluoromethoxy-4-methoxyphenyl)-5-ethyl-2,3,4,5-tetrahydropyridazin-3-one;

2-(4-methoxalylaminobenzyl)-6-(3-fluoromethoxy-4-methoxyphenyl)-5-ethyl-2,3,4,5-tetrahydropyridazin-3-one;

2-(4-ureidobenzyl)-6-(3-fluoromethoxy-4-methoxyphenyl)-5-ethyl-2,3,4,5-tetrahydropyridazin-3-one;

2-(4-pentanoylaminobenzyl)-6-(3-fluoromethoxy-4-methoxyphenyl)-5-ethyl-2,3,4,5-tetrahydropyridazin-3-one;

2-(4-hexanoylaminobenzyl)-6-(3-fluoromethoxy-4-methoxyphenyl)-5-ethyl-2,3,4,5-tetrahydropyridazin-3-one;

2-(4-pentafluoropropionylaminobenzyl)-6-(3-fluoromethoxy-4-methoxyphenyl)-5-ethyl-2,3,4,5-tetrahydropyridazin-3-one;

2-(4-acetamidobenzyl)-6-(3-difluoromethoxy-4-methoxyphenyl)-5-ethyl-2,3,4,5-tetrahydropyridazin-3-one;

2-(4-trifluoroacetamidobenzyl)-6-(3-difluoromethoxy-4-methoxyphenyl)-5-ethyl-2,3,4,5-tetrahydropyridazin-3-one;

2-(4-methylsulfonamidobenzyl)-6-(3-difluoromethoxy-4-methoxyphenyl)-5-ethyl-2,3,4,5-tetrahydropyridazin-3-one;

2-(4-propionylaminobenzyl)-6-(3-difluoromethoxy-4-methoxyphenyl)-5-ethyl-2,3,4,5-tetrahydropyridazin-3-one;

2-(4-butyrylaminobenzyl)-6-(3-difluoromethoxy-4-methoxyphenyl)-5-ethyl-2,3,4,5-tetrahydropyridazin-3-one;

2-(4-isobutyrylaminobenzyl)-6-(3-difluoromethoxy-4-methoxyphenyl)-5-ethyl-2,3,4,5-tetrahydropyridazin-3-one;

2-(4-methoxycarbonylaminobenzyl)-6-(3-difluoromethoxy-4-methoxyphenyl)-5-ethyl-2,3,4,5-tetrahydropyridazin-3-one;

2-(4-pivalylaminobenzyl)-6-(3-difluoromethoxy-4-methoxyphenyl)-5-ethyl-2,3,4,5-tetrahydropyridazin-3-one;

2-(4-cyclopentylcarbamoylbenzyl)-6-(3-difluoromethoxy-4-methoxyphenyl)-5-ethyl-2,3,4,5-tetrahydropyridazin-3-one;

2-(4-ethoxycarbonylaminobenzyl)-6-(3-difluoromethoxy-4-methoxyphenyl)-5-ethyl-2,3,4,5-tetrahydropyridazin-3-one;

2-(4-methoxalylaminobenzyl)-6-(3-difluoromethoxy-4-methoxyphenyl)-5-ethyl-2,3,4,5-tetrahydropyridazin-3-one;

2-(4-ureidobenzyl)-6-(3-difluoromethoxy-4-methoxyphenyl)-5-ethyl-2,3,4,5-tetrahydropyridazin-3-one;

2-(4-pentanoylaminobenzyl)-6-(3-difluoromethoxy-4-methoxyphenyl)-5-ethyl-2,3,4,5-tetrahydropyridazin-3-one;

2-(4-hexanoylaminobenzyl)-6-(3-difluoromethoxy-4-methoxyphenyl)-5-ethyl-2,3,4,5-tetrahydropyridazin-3-one;

2-(4-pentafluoropropionylaminobenzyl)-6-(3-difluoromethoxy-4-methoxyphenyl)-5-ethyl-2,3,4,5-tetrahydropyridazin-3-one;

2-(4-acetamidobenzyl)-6-(3-trifluoromethoxy-4-methoxyphenyl)-5-ethyl-2,3,4,5-tetrahydropyridazin-3-one;

2-(4-trifluoroacetamidobenzyl)-6-(3-trifluoromethoxy-4-methoxyphenyl)-5-ethyl-2,3,4,5-tetrahydropyridazin-3-one;

2-(4-methylsulfonamidobenzyl)-6-(3-trifluoromethoxy-4-methoxyphenyl)-5-ethyl-2,3,4,5-tetrahydropyridazin-3-one;

2-(4-propionylaminobenzyl)-6-(3-trifluoromethoxy-4-methoxyphenyl)-5-ethyl-2,3,4,5-tetrahydropyridazin-3-one;

2-(4-butyrylaminobenzyl)-6-(3-trifluoromethoxy-4-methoxyphenyl)-5-ethyl-2,3,4,5-tetrahydropyridazin-3-one;

2-(4-isobutyrylaminobenzyl)-6-(3-trifluoromethoxy-4-methoxyphenyl)-5-ethyl-2,3,4,5-tetrahydropyridazin-3-one;

2-(4-methoxycarbonylaminobenzyl)-6-(3-trifluoromethoxy-4-methoxyphenyl)-5-ethyl-2,3,4,5-tetrahydropyridazin-3-one;

2-(4-pivalylaminobenzyl)-6-(3-trifluoromethoxy-4-methoxyphenyl)-5-ethyl-2,3,4,5-tetrahydropyridazin-3-one;

2-(4-cyclopentylcarbamoylbenzyl)-6-(3-trifluoromethoxy-4-methoxyphenyl)-5-ethyl-2,3,4,5-tetrahydropyridazin-3-one;

2-(4-ethoxycarbonylaminobenzyl)-6-(3-trifluoromethoxy-4-methoxyphenyl)-5-ethyl-2,3,4,5-tetrahydropyridazin-3-one;

2-(4-methoxalylaminobenzyl)-6-(3-trifluoromethoxy-4-methoxyphenyl)-5-ethyl-2,3,4,5-tetrahydropyridazin-3-one;

2-(4-ureidobenzyl)-6-(3-trifluoromethoxy-4-methoxyphenyl)-5-ethyl-2,3,4,5-tetrahydropyridazin-3-one;

2-(4-pentanoylaminobenzyl)-6-(3-trifluoromethoxy-4-methoxyphenyl)-5-ethyl-2,3,4,5-tetrahydropyridazin-3-one;

2-(4-hexanoylaminobenzyl)-6-(3-trifluoromethoxy-4-methoxyphenyl)-5-ethyl-2,3,4,5-tetrahydropyridazin-3-one;

2-(4-pentafluoropropionylaminobenzyl)-6-(3-trifluoromethoxy-4-methoxyphenyl)-5-ethyl-2,3,4,5-tetrahydropyridazin-3-one;

2-(4-acetamidobenzyl)-6-(3-methoxy-4-fluoromethoxyphenyl)-5-ethyl-2,3,4,5-tetrahydropyridazin-3-one;

2-(4-trifluoroacetamidobenzyl)-6-(3-methoxy-4-fluoromethoxyphenyl)-5-ethyl-2,3,4,5-tetrahydropyridazin-3-one;

2-(4-methylsulfonamidobenzyl)-6-(3-methoxy-4-fluoromethoxyphenyl)-5-ethyl-2,3,4,5-tetrahydropyridazin-3-one;

2-(4-propionylaminobenzyl)-6-(3-methoxy-4-fluoromethoxyphenyl)-5-ethyl-2,3,4,5-tetrahydropyridazin-3-one;

2-(4-butyrylaminobenzyl)-6-(3-methoxy-4-fluoromethoxyphenyl)-5-ethyl-2,3,4,5-tetrahydropyridazin-3-one;

2-(4-isobutyrylaminobenzyl)-6-(3-methoxy-4-fluoromethoxyphenyl)-5-ethyl-2,3,4,5-tetrahydropyridazin-3-one;

2-(4-methoxycarbonylaminobenzyl)-6-(3-methoxy-4-fluoromethoxyphenyl)-5-ethyl-2,3,4,5-tetrahydropyridazin-3-one;

2-(4-pivalylaminobenzyl)-6-(3-methoxy-4-fluoromethoxyphenyl)-5-ethyl-2,3,4,5-tetrahydropyridazin-3-one;

2-(4-cyclopentylcarbamoylbenzyl)-6-(3-methoxy-4-fluoromethoxyphenyl)-5-ethyl-2,3,4,5-tetrahydropyridazin-3-one;

2-(4-ethoxycarbonylaminobenzyl)-6-(3-methoxy-4-fluoromethoxyphenyl)-5-ethyl-2,3,4,5-tetrahydropyridazin-3-one;

2-(4-methoxalylaminobenzyl)-6-(3-methoxy-4-fluoromethoxyphenyl)-5-ethyl-2,3,4,5-tetrahydropyridazin-3-one;

2-(4-ureidobenzyl)-6-(3-methoxy-4-fluoromethoxyphenyl)-5-ethyl-2,3,4,5-tetrahydropyridazin-3-one;

2-(4-pentanoylaminobenzyl)-6-(3-methoxy-4-fluoromethoxyphenyl)-5-ethyl-2,3,4,5-tetrahydropyridazin-3-one;

2-(4-hexanoylaminobenzyl)-6-(3-methoxy-4-fluoromethoxyphenyl)-5-ethyl-2,3,4,5-tetrahydropyridazin-3-one;

2-(4-pentafluoropropionylaminobenzyl)-6-(3-methoxy-4-fluoromethoxyphenyl)-5-ethyl-2,3,4,5-tetrahydropyridazin-3-one;

2-(4-acetamidobenzyl)-6-(3-methoxy-4-difluoromethoxyphenyl)-5-ethyl-2,3,4,5-tetrahydropyridazin-3-one;

2-(4-trifluoroacetamidobenzyl)-6-(3-methoxy-4-difluoromethoxyphenyl)-5-ethyl-2,3,4,5-tetrahydropyridazin-3-one;

2-(4-methylsulfonamidobenzyl)-6-(3-methoxy-4-difluoromethoxyphenyl)-5-ethyl-2,3,4,5-tetrahydropyridazin-3-one;

2-(4-propionylaminobenzyl)-6-(3-methoxy-4-difluoromethoxyphenyl)-5-ethyl-2,3,4,5-tetrahydropyridazin-3-one;

2-(4-butyrylaminobenzyl)-6-(3-methoxy-4-difluoromethoxyphenyl)-5-ethyl-2,3,4,5-tetrahydropyridazin-3-one;

2-(4-isobutyrylaminobenzyl)-6-(3-methoxy-4-difluoromethoxyphenyl)-5-ethyl-2,3,4,5-tetrahydropyridazin-3-one;

2-(4-methoxycarbonylaminobenzyl)-6-(3-methoxy-4-difluoromethoxyphenyl)-5-ethyl-2,3,4,5-tetrahydropyridazin-3-one;

2-(4-pivalylaminobenzyl)-6-(3-methoxy-4-difluoromethoxyphenyl)-5-ethyl-2,3,4,5-tetrahydropyridazin-3-one;

2-(4-cyclopentylcarbamoylbenzyl)-6-(3-methoxy-4-difluoromethoxyphenyl)-5-ethyl-2,3,4,5-tetrahydropyridazin-3-one;

2-(4-ethoxycarbonylaminobenzyl)-6-(3-methoxy-4-difluoromethoxyphenyl)-5-ethyl-2,3,4,5-tetrahydropyridazin-3-one;

2-(4-methoxalylaminobenzyl)-6-(3-methoxy-4-difluoromethoxyphenyl)-5-ethyl-2,3,4,5-tetrahydropyridazin-3-one;

2-(4-ureidobenzyl)-6-(3-methoxy-4-difluoromethoxyphenyl)-5-ethyl-2,3,4,5-tetrahydropyridazin-3-one;

2-(4-pentanoylaminobenzyl)-6-(3-methoxy-4-difluoromethoxyphenyl)-5-ethyl-2,3,4,5-tetrahydropyridazin-3-one;

2-(4-hexanoylaminobenzyl)-6-(3-methoxy-4-difluoromethoxyphenyl)-5-ethyl-2,3,4,5-tetrahydropyridazin-3-one;

2-(4-pentafluoropropionylaminobenzyl)-6-(3-methoxy-4-difluoromethoxyphenyl)-5-ethyl-2,3,4,5-tetrahydropyridazin-3-one;

2-(4-acetamidobenzyl)-6-(3-methoxy-4-trifluoromethoxyphenyl)-5-ethyl-2,3,4,5-tetrahydropyridazin-3-one;

2-(4-trifluoroacetamidobenzyl)-6-(3-methoxy-4-trifluoromethoxyphenyl)-5-ethyl-2,3,4,5-tetrahydropyridazin-3-one;

2-(4-methylsulfonamidobenzyl)-6-(3-methoxy-4-trifluoromethoxyphenyl)-5-ethyl-2,3,4,5-tetrahydropyridazin-3-one;

2-(4-propionylaminobenzyl)-6-(3-methoxy-4-trifluoromethoxyphenyl)-5-ethyl-2,3,4,5-tetrahydropyridazin-3-one;

2-(4-butyrylaminobenzyl)-6-(3-methoxy-4-trifluoromethoxyphenyl)-5-ethyl-2,3,4,5-tetrahydropyridazin-3-one;

2-(4-isobutyrylaminobenzyl)-6-(3-methoxy-4-trifluoromethoxyphenyl)-5-ethyl-2,3,4,5-tetrahydropyridazin-3-one;

2-(4-methoxycarbonylaminobenzyl)-6-(3-methoxy-4-trifluoromethoxyphenyl)-5-ethyl-2,3,4,5-tetrahydropyridazin-3-one;

2-(4-pivalylaminobenzyl)-6-(3-methoxy-4-trifluoromethoxyphenyl)-5-ethyl-2,3,4,5-tetrahydropyridazin-3-one;

2-(4-cyclopentylcarbamoylbenzyl)-6-(3-methoxy-4-trifluoromethoxyphenyl)-5-ethyl-2,3,4,5-tetrahydropyridazin-3-one;

2-(4-ethoxycarbonylaminobenzyl)-6-(3-methoxy-4-trifluoromethoxyphenyl)-5-ethyl-2,3,4,5-tetrahydropyridazin-3-one;

2-(4-methoxalylaminobenzyl)-6-(3-methoxy-4-trifluoromethoxyphenyl)-5-ethyl-2,3,4,5-tetrahydropyridazin-3-one;

2-(4-ureidobenzyl)-6-(3-methoxy-4-trifluoromethoxyphenyl)-5-ethyl-2,3,4,5-tetrahydropyridazin-3-one;

2-(4-pentanoylaminobenzyl)-6-(3-methoxy-4-trifluoromethoxyphenyl)-5-ethyl-2,3,4,5-tetrahydropyridazin-3-one;

2-(4-hexanoylaminobenzyl)-6-(3-methoxy-4-trifluoromethoxyphenyl)-5-ethyl-2,3,4,5-tetrahydropyridazin-3-one;

2-(4-pentafluoropropionylaminobenzyl)-6-(3-methoxy-4-trifluoromethoxyphenyl)-5-ethyl-2,3,4,5-tetrahydropyridazin-3-one;

2-(4-acetamidobenzyl)-6-(3-ethoxy-4-methoxyphenyl)-2,3,4,5-tetrahydropyridazin-3-one, m.p. 170°;

2-(4-trifluoroacetamidobenzyl)-6-(3-ethoxy-4-methoxyphenyl)-2,3,4,5-tetrahydropyridazin-3-one;

2-(4-methylsulfonamidobenzyl)-6-(3-ethoxy-4-methoxyphenyl)-2,3,4,5-tetrahydropyridazin-3-one;

2-(4-propionylaminobenzyl)-6-(3-ethoxy-4-methoxyphenyl)-2,3,4,5-tetrahydropyridazin-3-one;

2-(4-butyrylaminobenzyl)-6-(3-ethoxy-4-methoxyphenyl)-2,3,4,5-tetrahydropyridazin-3-one;

2-(4-isobutyrylaminobenzyl)-6-(3-ethoxy-4-methoxyphenyl)-2,3,4,5-tetrahydropyridazin-3-one;

2-(4-methoxycarbonylaminobenzyl)-6-(3-ethoxy-4-methoxyphenyl)-2,3,4,5-tetrahydropyridazin-3-one;

2-(4-pivalylaminobenzyl)-6-(3-ethoxy-4-methoxyphenyl)-2,3,4,5-tetrahydropyridazin-3-one;

2-(4-cyclopentylcarbamoylbenzyl)-6-(3-ethoxy-4-methoxyphenyl)-2,3,4,5-tetrahydropyridazin-3-one;

2-(4-ethoxycarbonylaminobenzyl)-6-(3-ethoxy-4-methoxyphenyl)-2,3,4,5-tetrahydropyridazin-3-one, m.p. 128°;

2-(4-methoxalylaminobenzyl)-6-(3-ethoxy-4-methoxyphenyl)-2,3,4,5-tetrahydropyridazin-3-one;

2-(4-ureidobenzyl)-6-(3-ethoxy-4-methoxyphenyl)-2,3,4,5-tetrahydropyridazin-3-one;

2-(4-pentanoylaminobenzyl)-6-(3-ethoxy-4-methoxyphenyl)-2,3,4,5-tetrahydropyridazin-3-one;

2-(4-hexanoylaminobenzyl)-6-(3-ethoxy-4-methoxyphenyl)-2,3,4,5-tetrahydropyridazin-3-one;

2-(4-pentafluoropropionylaminobenzyl)-6-(3-ethoxy-4-methoxyphenyl)-2,3,4,5-tetrahydropyridazin-3-one;

2-(4-acetamidobenzyl)-6-(3-cyclopentyloxy-4-methoxyphenyl)-2,3,4,5-tetrahydropyridazin-3-one;

2-(4-trifluoroacetamidobenzyl)-6-(3-cyclopentyloxy-4-methoxyphenyl)-2,3,4,5-tetrahydropyridazin-3-one;

2-(4-methylsulfonamidobenzyl)-6-(3-cyclopentyloxy-4-methoxyphenyl)-2,3,4,5-tetrahydropyridazin-3-one;

2-(4-propionylaminobenzyl)-6-(3-cyclopentyloxy-4-methoxyphenyl)-2,3,4,5-tetrahydropyridazin-3-one;

2-(4-tert-butylcarbonylaminobenzyl)-6-(3-cyclopentyloxy-4-methoxyphenyl)-2,3,4,5-tetrahydropyridazin-3-one;

2-(4-butyrylaminobenzyl)-6-(3-cyclopentyloxy-4-methoxyphenyl)-2,3,4,5-tetrahydropyridazin-3-one;

2-(4-isobutyrylaminobenzyl)-6-(3-cyclopentyloxy-4-methoxyphenyl)-2,3,4,5-tetrahydropyridazin-3-one;

2-(4-methoxycarbonylaminobenzyl)-6-(3-cyclopentyloxy-4-methoxyphenyl)-2,3,4,5-tetrahydropyridazin-3-one;

2-(4-pivalylaminobenzyl)-6-(3-cyclopentyloxy-4-methoxyphenyl)-2,3,4,5-tetrahydropyridazin-3-one;

2-(4-cyclopentylcarbamoylbenzyl)-6-(3-cyclopentyloxy-4-methoxyphenyl)-2,3,4,5-tetrahydropyridazin-3-one;

2-(4-ethoxycarbonylaminobenzyl)-6-(3-cyclopentyloxy-4-methoxyphenyl)-2,3,4,5-tetrahydropyridazin-3-one;

2-(4-methoxalylaminobenzyl)-6-(3-cyclopentyloxy-4-methoxyphenyl)-2,3,4,5-tetrahydropyridazin-3-one;

2-(4-ureidobenzyl)-6-(3-cyclopentyloxy-4-methoxyphenyl)-2,3,4,5-tetrahydropyridazin-3-one;

2-(4-pentanoylaminobenzyl)-6-(3-cyclopentyloxy-4-methoxyphenyl)-2,3,4,5-tetrahydropyridazin-3-one;

2-(4-hexanoylaminobenzyl)-6-(3-cyclopentyloxy-4-methoxyphenyl)-2,3,4,5-tetrahydropyridazin-3-one;

2-(4-pentafluoropropionylaminobenzyl)-6-(3-cyclopentyloxy-4-methoxyphenyl)-2,3,4,5-tetrahydropyridazin-3-one;

2-(4-acetamidophenethyl)-6-(3,4-dimethoxyphenyl)-2,3,4,5-tetrahydropyridazin-3-one;

2-(4-trifluoroacetamidophenethyl)-6-(3,4-dimethoxyphenyl)-2,3,4,5-tetrahydropyridazin-3-one;

2-(4-methylsulfonamidophenethyl)-6-(3,4-dimethoxyphenyl)-2,3,4,5-tetrahydropyridazin-3-one;

2-(4-propionylaminophenethyl)-6-(3,4-dimethoxyphenyl)-2,3,4,5-tetrahydropyridazin-3-one;

2-(4-butyrylaminophenethyl)-6-(3,4-dimethoxyphenyl)-2,3,4,5-tetrahydropyridazin-3-one;

2-(4-isobutyrylaminophenethyl)-6-(3,4-dimethoxyphenyl)-2,3,4,5-tetrahydropyridazin-3-one;

2-(4-methoxycarbonylaminophenethyl)-6-(3,4-dimethoxyphenyl)-2,3,4,5-tetrahydropyridazin-3-one;

2-(4-pivalylaminophenethyl)-6-(3,4-dimethoxyphenyl)-2,3,4,5-tetrahydropyridazin-3-one;

2-(4-cyclopentylcarbamoylphenethyl)-6-(3,4-dimethoxyphenyl)-2,3,4,5-tetrahydropyridazin-3-one;

2-(4-ethoxycarbonylaminophenethyl)-6-(3,4-dimethoxyphenyl)-2,3,4,5-tetrahydropyridazin-3-one;

2-(4-methoxalylaminophenethyl)-6-(3,4-dimethoxyphenyl)-2,3,4,5-tetrahydropyridazin-3-one;

2-(4-ureidophenethyl)-6-(3,4-dimethoxyphenyl)-2,3,4,5-tetrahydropyridazin-3-one;

2-(4-pentanoylaminophenethyl)-6-(3,4-dimethoxyphenyl)-2,3,4,5-tetrahydropyridazin-3-one;

2-(4-hexanoylaminophenethyl)-6-(3,4-dimethoxyphenyl)-2,3,4,5-tetrahydropyridazin-3-one;

2-(4-pentafluoropropionylaminophenethyl)-6-(3,4-dimethoxyphenyl)-2,3,4,5-tetrahydropyridazin-3-one;

2-(3-acetamidobenzyl)-6-(3,4-dimethoxyphenyl)-2,3,4,5-tetrahydropyridazin-3-one, m.p. 105°;

2-(3-trifluoroacetamidobenzyl)-6-(3,4-dimethoxyphenyl)-2,3,4,5-tetrahydropyridazin-3-one, m.p. 136°;

2-(3-methylsulfonamidobenzyl)-6-(3,4-dimethoxyphenyl)-2,3,4,5-tetrahydropyridazin-3-one, m.p. 177°;

2-(3-propionylaminobenzyl)-6-(3,4-dimethoxyphenyl)-2,3,4,5-tetrahydropyridazin-3-one, m.p. 152°;

2-(3-tert-butylcarbonylaminobenzyl)-6-(3,4-dimethoxyphenyl)-2,3,4,5-tetrahydropyridazin-3-one;

2-(3-butyrylaminobenzyl)-6-(3,4-dimethoxyphenyl)-2,3,4,5-tetrahydropyridazin-3-one;

2-(3-isobutyrylaminobenzyl)-6-(3,4-dimethoxyphenyl)-2,3,4,5-tetrahydropyridazin-3-one;

2-(3-methoxycarbonylaminobenzyl)-6-(3,4-dimethoxyphenyl)-2,3,4,5-tetrahydropyridazin-3-one;

2-(3-pivalylaminobenzyl)-6-(3,4-dimethoxyphenyl)-2,3,4,5-tetrahydropyridazin-3-one;

2-(3-cyclopentylcarbamoylbenzyl)-6-(3,4-dimethoxyphenyl)-2,3,4,5-tetrahydropyridazin-3-one;

2-(3-ethoxycarbonylaminobenzyl)-6-(3,4-dimethoxyphenyl)-2,3,4,5-tetrahydropyridazin-3-one, m.p. 179°;

2-(3-methoxalylaminobenzyl)-6-(3,4-dimethoxyphenyl)-2,3,4,5-tetrahydropyridazin-3-one;

2-(3-ureidobenzyl)-6-(3,4-dimethoxyphenyl)-2,3,4,5-tetrahydropyridazin-3-one;

2-(3-pentanoylaminobenzyl)-6-(3,4-dimethoxyphenyl)-2,3,4,5-tetrahydropyridazin-3-one;

2-(3-hexanoylaminobenzyl)-6-(3,4-dimethoxyphenyl)-2,3,4,5-tetrahydropyridazin-3-one;

2-(3-pentafluoropropionylaminobenzyl)-6-(3,4-dimethoxyphenyl)-2,3,4,5-tetrahydropyridazin-3-one;

2-(3-acetamidobenzyl)-6-(3-cyclopentyloxy-4-methoxyphenyl)-5-ethyl-2,3,4,5-tetrahydropyridazin-3-one;

2-(3-trifluoroacetamidobenzyl)-6-(3-cyclopentyloxy-4-methoxyphenyl)-5-ethyl-2,3,4,5-tetrahydropyridazin-3-one, m.p. 70°;

2-(3-methylsulfonamidobenzyl)-6-(3-cyclopentyloxy-4-methoxyphenyl)-5-ethyl-2,3,4,5-tetrahydropyridazin-3-one;

2-(3-propionylaminobenzyl)-6 (3-cyclopentyloxy-4-methoxyphenyl)-5-ethyl-2,3,4,5-tetrahydropyridazin-3-one, m.p. 113°;

2-(3-tert-butylcarbonylaminobenzyl)-6-(3-cyclopentyloxy-4-methoxyphenyl)-5-ethyl-2,3,4,5-tetrahydropyridazin-3-one;

2-(3-butyrylaminobenzyl)-6-(3-cyclopentyloxy-4-methoxyphenyl)-5-ethyl-2,3,4,5-tetrahydropyridazin-3-one;

2-(3-isobutyrylaminobenzyl)-6-(3-cyclopentyloxy-4-methoxyphenyl)-5-ethyl-2,3,4,5-tetrahydropyridazin-3-one;

2-(3-methoxycarbonylaminobenzyl)-6-(3-cyclopentyloxy-4-methoxyphenyl)-5-ethyl-2,3,4,5-tetrahydropyridazin-3-one;

2-(3-pivalylaminobenzyl)-6-(3-cyclopentyloxy-4-methoxyphenyl)-5-ethyl-2,3,4,5-tetrahydropyridazin-3-one;

2-(3-cyclopentylcarbamoylbenzyl)-6-(3-cyclopentyloxy-4-methoxyphenyl)-5-ethyl-2,3,4,5-tetrahydropyridazin-3-one;

2-(3-ethoxycarbonylaminobenzyl)-6-(3-cyclopentyloxy-4-methoxyphenyl)-5-ethyl-2,3,4,5-tetrahydropyridazin-3-one, m.p. 153°;

2-(3-methoxalylaminobenzyl)-6-(3-cyclopentyloxy-4-methoxyphenyl)-5-ethyl-2,3,4,5-tetrahydropyridazin-3-one;

2-(3-ureidobenzyl)-6-(3-cyclopentyloxy-4-methoxyphenyl)-5-ethyl-2,3,4,5-tetrahydropyridazin-3-one;

2-(3-pentanoylaminobenzyl)-6-(3-cyclopentyloxy-4-methoxyphenyl)-5-ethyl-2,3,4,5-tetrahydropyridazin-3-one;

2-(3-hexanoylaminobenzyl)-6-(3-cyclopentyloxy-4-methoxyphenyl)-5-ethyl-2,3,4,5-tetrahydropyridazin-3-one;

2-(3-pentafluoropropionylaminobenzyl)-6-(3-cyclopentyloxy-4-methoxyphenyl)-5-ethyl-2,3,4,5-tetrahydropyridazin-3-one;

2-(4-nitrobenzyl)-6-(3,4-dimethoxyphenyl)-2,3,4,5-tetrahydropyridazin-3-one, m.p. 126°;

2-(4-nitrophenyl)-6-(3,4-dimethoxyphenyl)-2,3,4,5-tetrahydropyridazin-3-one;

b) compounds, disclosed in EP 0 763 534,selected from the group consisting of

2-(4-nicotinoylaminobenzyl)-6-(3,4-dimethoxyphenyl)-2,3,4,5-tetrahydropyridazin-3-one;

2-(3-nicotinoylaminobenzyl)-6-(3,4-dimethoxyphenyl)-2,3,4,5-tetrahydropyridazin-3-one;

2-(2-nicotinoylaminobenzyl)-6-(3,4-dimethoxyphenyl)-2,3,4,5-tetrahydropyridazin-3-one;

2-(4-nicotinoylaminobenzyl)-6-(3,4-dimethoxyphenyl)-5-ethyl-2,3,4,5-tetrahydropyridazin-3-one;

2-(3-nicotinoylaminobenzyl)-6-(3,4-dimethoxyphenyl)-5-ethyl-2,3,4,5-tetrahydropyridazin-3-one;

2-(2-nicotinoylaminobenzyl)-6-(3,4-dimethoxyphenyl)-5-ethyl-2,3,4,5-tetrahydropyridazin-3-one;

2-(4-nicotinoylaminobenzyl)-6-(3-methoxy-4-trifluoromethoxyphenyl)-5-ethyl-2,3,4,5-tetrahydropyridazin-3-one;

2-(4-nicotinoylaminobenzyl)-6-(3-methoxy-4-difluoromethoxyphenyl)-5-ethyl-2,3,4,5-tetrahydropyridazin-3-one;

2-(4-nicotinoylaino-benzyl)-6-(3-methoxy-4-fluoromethoxyphenyl)-5-ethyl-2,3,4,5-tetrahydropyridazin-3-one;

2-(4-nicotinoylaminobenzyl)-6-(3-difluoromethoxy-4-methoxyphenyl)-5-ethyl-2,3,4,5-tetrahydropyridazin-3-one;

2-(4-nicotinoylaminobenzyl)-6-(3-trifluoromethoxy-4-methoxyphenyl)-5-ethyl-2,3,4,5-tetrahydropyridazin-3-one;

2-(4-nicotinoylaminobenzyl)-6-(3-fluoromethoxy-4-methoxyphenyl)-5-ethyl-2,3,4,5-tetrahydropyridazin-3-one;

2-(4-nicotinoylaminobenzyl)-6-(3-methoxy-4-ethoxyphenyl)-5-ethyl-2,3,4,5-tetrahydropyridazin-3-one;

2-(4-nicotinoylaminobenzyl)-6-(3-ethoxy-4-methoxyphenyl)-5-ethyl-2,3,4,5-tetrahydropyridazin-3-one;

2-(4-nicotinoylaminobenzyl)-6-(3-hydroxy-4-methoxyphenyl)-5-ethyl-2,3,4,5-tetrahydropyridazin-3-one;

2-(4-nicotinoylaminobenzyl)-6-(4-methylsulfonylphenyl)-5-ethyl-2,3,4,5-tetrahydropridazin-3-one;

2-(4-nicotinoylaminobenzyl)-6-(4-methyleneoxyphenyl)-5-ethyl-2,3,4,5-tetrahydropyridazin-3-one;

2-(4-nicotinoylaminobenzyl)-6-(3-cyclopentyloxy-4-methoxyphenyl)-5-ethyl-2,3,4,5-tetrahydropyridazin-3-one;

2-(3-nicotinoylaminobenzyl)-6-(3-cyclopentyloxy-4-methoxyphenyl)-5-ethyl-2,3,4,5-tetrahydropyridazin-3-one;

2-(4-nicotinoylaminophenethyl)-6-(3,4-dimethoxyphenyl)-2,3,4,5-tetrahydropyridazin-3-one;

2-(4-nicotinoylaminophenethyl)-6-(3,4-dimethoxyphenyl)-5-ethyl-2,3,4,5-tetrahydropyridazin-3-one;

3-(4-nicotinoylaminobenzyl)-5-(3,4-dimethoxyphenyl)-3,6-dihydro-1,3,4-thiadiazin-2-one;

3-(3-nicotinoylaminobenzyl)-5-(3,4-dimethoxyphenyl)-3,6-dihydro-1,3,4-thiadiazin-2-one;

3-(2-nicotinoylaminobenzyl)-5-(3,4-dimethoxyphenyl)-3,6-dihydro-1,3,4-thiadiazin-2-one;

3-(4-nicotinoylaminobenzyl)-5-(3,4-dimethoxyphenyl)-6-ethyl-3,6-dihydro-1,3,4-thiadiazin-2-one;

3-(3-nicotinoylaminobenzyl)-5-(3,4-dimethoxyphenyl)-6-ethyl-3,6-dihydro-1,3,4-thiadiazin-2-one;

3-(2-nicotinoylaminobenzyl)-5-(3,4-dimethoxyphenyl)-6-ethyl-3,6-dihydro-1,3,4-thiadiazin-2-one;

3-(4-nicotinoylaminobenzyl)-5-(3-methoxy-4-trifluoromethoxyphenyl)-6-ethyl-3,6-dihydro-1,3,4-thiadiazin-2-one;

3-(4-nicotinoylaminobenzyl)-5-(3-methoxy-4-difluoromethoxyphenyl)-6-ethyl-3,6-dihydro-1,3,4-thiadiazin-2-one;

3-(4-nicotinoylaminobenzyl)-5-(3-methoxy-4-fluoromethoxyphenyl)-6-ethyl-3,6-dihydro-1,3,4-thiadiazin-2-one;

3-(4-nicotinoylaminobenzyl)-5-(3-difluoromethoxy-4-methoxyphenyl)-6-ethyl-3,6-dihydro-1,3,4-thiadiazin-2-one;

3-(4-nicotinoylaminobenzyl)-5-(3-trifluoromethoxy-4-methoxyphenyl)-6-ethyl-3,6-dihydro-1,3,4-thiadiazin-2-one;

3-(4-nicotinoylaminobenzyl)-5-(3-fluoromethoxy-4-methoxyphenyl)-6-ethyl-3,6-dihydro-1,3,4-thiadiazin-2-one;

3-(4-nicotinoylaminobenzyl)-5-(3-methoxy-4-ethoxyphenyl)-6-ethyl-3,6-dihydro-1,3,4-thiadiazin-2-one;

3-(4-nicotinoylaminobenzyl)-5-(3-ethoxy-4-methoxyphenyl)-6-ethyl-3,6-dihydro-1,3,4-thiadiazin-2-one;

3-(4-nicotinoylaminobenzyl)-5-(3-hydroxy-4-methoxyphenyl)-6-ethyl-3,6-dihydro-1,3,4-thiadiazin-2-one;

3-(4-nicotinoylaminobenzyl)-5-(4-methylsulfonylphenyl)-6-ethyl-3,6-dihydro-1,3,4-thiadiazin-2-one;

3-(4-nicotinoylaminobenzyl)-5-(4-methyleneoxyphenyl)-6-ethyl-3,6-dihydro-1,3,4-thiadiazin-2-one;

3-(4-nicotinoylaminobenzyl)-5-(3-cyclopentyloxy-4-methoxyphenyl)-6-ethyl-3,6-dihydro-1,3,4-thiadiazin-2-one;

3-(3-nicotinoylaminobenzyl)-5-(3-cyclopentyloxy-4-methoxyphenyl)-6-ethyl-3,6-dihydro-1,3,4-thiadiazin-2-one;

3-(4-nicotinoylamino-phenethyl)-5-(3,4-dimethoxyphenyl)-3,6-dihydro-1,3,4-thiadiazin-2-one;

3-(4-nicotinoylaminophenethyl)-5-(3,4-dimethoxyphenyl)-6-ethyl-3,6-dihydro-1,3,4-thiadiazin-2-one;

3-(4-nicotinoylaminobenzyl)-5-(3,4-dimethoxyphenyl)-3,6-dihydro-1,3,4-oxadiazin-2-one;

3-(3-nicotinoylaminobenzyl)-5-(3,4-dimethoxyphenyl)-3,6-dihydro-1,3,4-oxadiazin-2-one;

3-(2-nicotinoylaminobenzyl)-5-(3,4-dimethoxyphenyl)-3,6-dihydro-1,3,4-oxadiazin-2-one;

3-(4-nicotinoylaminobenzyl)-5-(3,4-dimethoxyphenyl)-6-ethyl-3,6-dihydro-1,3,4-oxadiazin-2-one;

3-(3-nicotinoylaminobenzyl)-5-(3,4-dimethoxyphenyl)-6-ethyl-3,6-dihydro-1,3,4-oxadiazin-2-one;

3-(2-nicotinoylaminobenzyl)-5-(3,4-dimethoxyphenyl)-6-ethyl-3,6-dihydro-1,3,4-oxadiazin-2-one;

3-(4-nicotinoylaminobenzyl)-5-(3-methoxy-4-trifluoromethoxyphenyl)-6-ethyl-3,6-dihydro-1,3,4-oxadiazin-2-one;

3-(4-nicotinoylaminobenzyl)-5-(3-methoxy-4-difluoromethoxyphenyl)-6-ethyl-3,6-dihydro-1,3,4-oxadiazin-2-one;

3-(4-nicotinoylaminobenzyl)-5-(3-methoxy-4-fluoromethoxyphenyl)-6-ethyl-3,6-dihydro-1,3,4-oxadiazin-2-one;

3-(4-nicotinoylaminobenzyl)-5-(3-difluoromethoxy-4-methoxyphenyl)-6-ethyl-3,6-dihydro-1,3,4-oxadiazin-2-one;

3-(4-nicotinoylaminobenzyl)-5-(3-trifluoromethoxy-4-methoxyphenyl)-6-ethyl-3,6-dihydro-1,3,4-oxadiazin-2-one;

3-(4-nicotinoylaminobenzyl)-5-(3-fluoromethoxy-4-methoxyphenyl)-6-ethyl-3,6-dihydro-1,3,4-oxadiazin-2-one;

3-(4-nicotinoylaminobenzyl)-5-(3-methoxy-4-ethoxyphenyl)-6-ethyl-3,6-dihydro-1,3,4-oxadiazin-2-one;

3-(4-nicotinoylaminobenzyl)-5-(3-ethoxy-4-methoxyphenyl)-6-ethyl-3,6-dihydro-1,3,4-oxadiazin-2-one;

3-(4-nicotinoylaminobenzyl)-5-(3-hydroxy-4-methoxyphenyl)-6-ethyl-3,6-dihydro-1,3,4-oxadiazin-2-one;

3-(4-nicotinoylaminobenzyl)-5-(4-methylsulfonylphenyl)-6-ethyl-3,6-dihydro-1,3,4-oxadiazin-2-one;

3-(4-nicotinoylaminobenzyl)-5-(4-methyleneoxyphenyl)-6-ethyl-3,6-dihydro-1,3,4-oxadiazin-2-one;

3-(4-nicotinoylaminobenzyl)-5-(3-cyclopentyloxy-4-methoxyphenyl)-6-ethyl-3,6-dihydro-1,3,4-oxadiazin-2-one;

3-(3-nicotinoylaminobenzyl)-5-(3-cyclopentyloxy-4-methoxyphenyl)-6-ethyl-3,6-dihydro-1,3,4-oxadiazin-2-one;

3-(4-nicotinoylamino-phenethyl)-5-(3,4-dimethoxyphenyl)-3,6-dihydro-1,3,4-oxadiazin-2-one;

3-(4-nicotinoylamino-phenethyl)-5-(3,4-dimethoxyphenyl)-6-ethyl-3,6-dihydro-1,3,4-oxadiazin-2-one;

2-(4-isonicotinoylaminobenzyl)-6-(3,4-dimethoxyphenyl)-2,3,4,5-tetrahydropyridazin-3-one;

2-(3-isonicotinoylaminobenzyl)-6-(3,4-dimethoxyphenyl)-2,3,4,5-tetrahydropyridazin-3-one;

2-(2-isonicotinoylaminobenzyl)-6-(3,4-dimethoxyphenyl)-2,3,4,5-tetrahydropyridazin-3-one;

2-(4-isonicotinoylaminobenzyl)-6-(3,4-dimethoxyphenyl)-5-ethyl-2,3,4,5-tetrahydropyridazin-3-one;

2-(3-isonicotinoylaminobenzyl)-6-(3,4-dimethoxyphenyl)-5-ethyl-2,3,4,5-tetrahydropyridazin-3-one;

2-(2-isonicotinoylaminobenzyl)-6-(3,4-dimethoxyphenyl)-5-ethyl-2,3,4,5-tetrahydropyridazin-3-one;

2-(4-isonicotinoylaminobenzyl)-6-(3-methoxy-4-trifluoromethoxyphenyl)-5-ethyl-2,3,4,5-tetrahydropyridazin-3-one;

2-(4-isonicotinoylaminobenzyl)-6-(3-methoxy-4-difluoromethoxyphenyl)-5-ethyl-2,3,4,5-tetrahydropyridazin-3-one;

2-(4-isonicotinoylaminobenzyl)-6-(3-methoxy-4-fluoromethoxyphenyl)-5-ethyl-2,3,4,5-tetrahydropyridazin-3-one;

2-(4-isonicotinoylaminobenzyl)-6-(3-difluoromethoxy-4-methoxyphenyl)-5-ethyl-2,3,4,5-tetrahydropyridazin-3-one;

2-(4-isonicotinoylaminobenzyl)-6-(3-trifluoromethoxy-4-methoxyphenyl)-5-ethyl-2,3,4,5-tetrahydropyridazin-3-one;

2-(4-isonicotinoylaminobenzyl)-6-(3-fluoromethoxy-4-methoxyphenyl)-5-ethyl-2,3,4,5-tetrahydropyridazin-3-one;

2-(4-isonicotinoylaminobenzyl)-6-(3-methoxy-4-ethoxyphenyl)-5-ethyl-2,3,4,5-tetrahydropyridazin-3-one;

2-(4-isonicotinoylaminobenzyl)-6-(3-ethoxy-4-methoxyphenyl)-5-ethyl-2,3,4,5-tetrahydropyridazin-3-one;

2-(4-isonicotinoylaminobenzyl)-6-(3-hydroxy-4-methoxyphenyl)-5-ethyl-2,3,4,5-tetrahydropyridazin-3-one;

2-(4-isonicotinoylaminobenzyl)-6-(4-methylsulfonylphenyl)-5-ethyl-2,3,4,5-tetrahydropyridazin-3-one;

2-(4-isonicotinoylaminobenzyl)-6-(4-methyleneoxyphenyl)-5-ethyl-2,3,4,5-tetrahydropyridazin-3-one;

2-(4-isonicotinoylaminobenzyl)-6-(3-cyclopentyloxy-4-methoxyphenyl)-5-ethyl-2,3,4,5-tetrahydropyridazin-3-one;

2-(3-isonicotinoylaminobenzyl)-6-(3-cyclopentyloxy-4-methoxyphenyl)-5-ethyl-2,3,4,5-tetrahydropyridazin-3-one;

2-(4-isonicotinoylaminophenethyl)-6-(3,4-dimethoxyphenyl)-2,3,4,5-tetrahydropyridazin-3-one;

2-(4-isonicotinoylaminophenethyl)-6-(3,4-dimethoxyphenyl)-5-ethyl-2,3,4,5-tetrahydropyridazin-3-one;

3-(4-isonicotinoylaminobenzyl)-5-(3,4-dimethoxyphenyl)-3,6-dihydro-1,3,4-thiadiazin-2-one;

3-(3-isonicotinoylaminobenzyl)-5-(3,4-dimethoxyphenyl)-3,6-dihydro-1,3,4-thiadiazin-2-one;

3-(2-isonicotinoylaminobenzyl)-5-(3,4-dimethoxyphenyl)-3,6-dihydro-1,3,4-thiadiazin-2-one;

3-(4-isonicotinoylaminobenzyl)-5-(3,4-dimethoxyphenyl)-6-ethyl-3,6-dihydro-1,3,4-thiadiazin-2-one;

3-(3-isonicotinoylaminobenzyl)-5-(3,4-dimethoxyphenyl)-6-ethyl-3,6-dihydro-1,3,4-thiadiazin-2-one;

3-(2-isonicotinoylaminobenzyl)-5-(3,4-dimethoxyphenyl)-6-ethyl-3,6-dihydro-1,3,4-thiadiazin-2-one;

3-(4-isonicotinoylaminobenzyl)-5-(3-methoxy-4-trifluoromethoxyphenyl)-6-ethyl-3,6-dihydro-1,3,4-thiadiazin-2-one;

3-(4-isonicotinoylaminobenzyl)-5-(3-methoxy-4-difluoromethoxyphenyl)-6-ethyl-3,6-dihydro-1,3,4-thiadiazin-2-one;

3-(4-isonicotinoylaminobenzyl)-5-(3-methoxy-4-fluoromethoxyphenyl)-6-ethyl-3,6-dihydro-1,3,4-thiadiazin-2-one;

3-(4-isonicotinoylaminobenzyl)-5-(3-difluoromethoxy-4-methoxyphenyl)-6-ethyl-3,6-dihydro-1,3,4-thiadiazin-2-one;

3-(4-isonicotinoylaminobenzyl)-5-(3-trifluoromethoxy-4-methoxyphenyl)-6-ethyl-3,6-dihydro-1,3,4-thiadiazin-2-one;

3-(4-nicotinoylaminobenzyl)-5-(3-fluoromethoxy-4-methoxyphenyl)-6-ethyl-3,6-dihydro-1,3,4-thiadiazin-2-one;

3-(4-isonicotinoylaminobenzyl)-5-(3-methoxy-4-ethoxyphenyl)-6-ethyl-3,6-dihydro-1,3,4-thiadiazin-2-one;

3-(4-isonicotinoylaminobenzyl)-5-(3-ethoxy-4-methoxyphenyl)-6-ethyl-3,6-dihydro-1,3,4-thiadiazin-2-one;

3-(4-isonicotinoylaminobenzyl)-5-(3-hydroxy-4-methoxyphenyl)-6-ethyl-3,6-dihydro-1,3,4-thiadiazin-2-one;

3-(4-isonicotinoylaminobenzyl)-5-(4-methylsulfonylphenyl)-6-ethyl-3,6-dihydro-1,3,4-thiadiazin-2-one;

3-(4-isonicotinoylaminobenzyl)-5-(4-methyleneoxyphenyl)-6-ethyl-3,6-dihydro-1,3,4-thiadiazin-2-one;

3-(4-isonicotinoylaminobenzyl)-5-(3-cyclopentyloxy-4-methoxyphenyl)-6-ethyl-3,6-dihydro-1,3,4-thiadiazin-2-one;

3-(3-isonicotinoylaminobenzyl)-5-(3-cyclopentyloxy-4-methoxyphenyl)-6-ethyl-3,6-dihydro-1,3,4-thiadiazin-2-one;

3-(4-isonicotinoylamino-phenethyl)-5-(3,4-dimethoxyphenyl)-3,6-dihydro-1,3,4-thiadiazin-2-one;

3-(4-isonicotinoylaminophenethyl)-5-(3,4-dimethoxyphenyl)-6-ethyl-3,6-dihydro-1,3,4-thiadiazin-2-one;

3-(4-isonicotinoylaminobenzyl)-5-(3,4-dimethoxyphenyl)-3,6-dihydro-1,3,4-oxadiazin-2-one;

3-(3-isonicotinoylaminobenzyl)-5-(3,4-dimethoxyphenyl)-3,6-dihydro-1,3,4-oxadiazin-2-one;

3-(2-isonicotinoylaminobenzyl)-5-(3,4-dimethoxyphenyl)-3,6-dihydro-1,3,4-oxadiazin-2-one;

3-(4-isonicotinoylaminobenzyl)-5-(3,4-dimethoxyphenyl)-6-ethyl-3,6-dihydro-1,3,4-oxadiazin-2-one;

3-(3-isonicotinoylaminobenzyl)-5-(3,4-dimethoxyphenyl)-6-ethyl-3,6-dihydro-1,3,4-oxadiazin-2-one;

3-(2-isonicotinoylaminobenzyl)-5-(3,4-dimethoxyphenyl)-6-ethyl-3,6-dihydro-1,3,4-oxadiazin-2-one;

3-(4-isonicotinoylaminobenzyl)-5-(3-methoxy-4-trifluoromethoxyphenyl)-6-ethyl-3,6-dihydro-1,3,4-oxadiazin-2-one;

3-(4-isonicotinoylaminobenzyl)-5-(3-methoxy-4-difluoromethoxyphenyl)-6-ethyl-3,6-dihydro-1,3,4-oxadiazin-2-one;

3-(4-isonicotinoylaminobenzyl)-5-(3-methoxy-4-fluoromethoxyphenyl)-6-ethyl-3,6-dihydro-1,3,4-oxadiazin-2-one;

3-(4-isonicotinoylaminobenzyl)-5-(3-difluoromethoxy-4-methoxyphenyl)-6-ethyl-3,6-dihydro-1,3,4-oxadiazin-2-one;

3-(4-isonicotinoylaminobenzyl)-5-(3-trifluoromethoxy-4-methoxyphenyl)-6-ethyl-3,6-dihydro-1,3,4-oxadiazin-2-one;

3-(4-isonicotinoylaminobenzyl)-5-(3-fluoromethoxy-4-methoxyphenyl)-6-ethyl-3,6-dihydro-1,3,4-oxadiazin-2-one;

3-(4-isonicotinoylaminobenzyl)-5-(3-methoxy-4-ethoxyphenyl)-6-ethyl-3,6-dihydro-1,3,4-oxadiazin-2-one;

3-(4-isonicotinoylaminobenzyl)-5-(3-ethoxy-4-methoxyphenyl)-6-ethyl-3,6-dihydro-1,3,4-oxadiazin-2-one;

3-(4-isonicotinoylaminobenzyl)-5-(3-hydroxy-4-methoxyphenyl)-6-ethyl-3,6-dihydro-1,3,4-oxadiazin-2-one;

3-(4-isonicotinoylaminobenzyl)-5-(4-methylsulfonylphenyl)-6-ethyl-3,6-dihydro-1,3,4-oxadiazin-2-one;

3-(4-isonicotinoylaminobenzyl)-5-(4-methyleneoxyphenyl)-6-ethyl-3,6-dihydro-1,3,4-oxadiazin-2-one;

3-(4-isonicotinoylaminobenzyl)-5-(3-cyclopentyloxy-4-methoxyphenyl)-6-ethyl-3,6-dihydro-1,3,4-oxadiazin-2-one;

3-(3-isonicotinoylaminobenzyl)-5-(3-cyclopentyloxy-4-methoxyphenyl)-6-ethyl-3,6-dihydro-1,3,4-oxadiazin-2-one;

3-(4-isonicotinoylamino-phenethyl)-5-(3,4-dimethoxyphenyl)-3,6-dihydro-1,3,4-oxadiazin-2-one;

3-(4-isonicotinoylamino-phenethyl)-5-(3,4-dimethoxyphenyl)-6-ethyl-3,6-dihydro-1,3,4-oxadiazin-2-one;

2-(4-picolinoylaminobenzyl)-6-(3,4-dimethoxyphenyl)-2,3,4,5-tetrahydropyridazin-3-one;

2-(3-picolinoylaminobenzyl)-6-(3,4-dimethoxyphenyl)-2,3,4,5-tetrahydropyridazin-3-one;

2-(2-picolinoylaminobenzyl)-6-(3,4-dimethoxyphenyl)-2,3,4,5-tetrahydropyridazin-3-one;

2-(4-picolinoylaminobenzyl)-6-(3,4-dimethoxyphenyl)-5-ethyl-2,3,4,5-tetrahydropyridazin-3-one;

2-(3-picolinoylaminobenzyl)-6-(3,4-dimethoxyphenyl)-5-ethyl-2,3,4,5-tetrahydropyridazin-3-one;

2-(2-picolinoylaminobenzyl)-6-(3,4-dimethoxyphenyl)-5-ethyl-2,3,4,5-tetrahydropyridazin-3-one;

2-(4-picolinoylaminobenzyl)-6-(3-methoxy-4-trifluoromethoxyphenyl)-5-ethyl-2,3,4,5-tetrahydropyridazin-3-one;

2-(4-picolinoylaminobenzyl)-6-(3-methoxy-4-difluoromethoxyphenyl)-5-ethyl-2,3,4,5-tetrahydropyridazin-3-one;

2-(4-picolinoylaminobenzyl)-6-(3-methoxy-4-fluoromethoxyphenyl)-5-ethyl-2,3,4,5-tetrahydropyridazin-3-one;

2-(4-picolinoylaminobenzyl)-6-(3-difluoromethoxy-4-methoxyphenyl)-5-ethyl-2,3,4,5-tetrahydropyridazin-3-one;

2-(4-picolinoylaminobenzyl)-6-(3-trifluoromethoxy-4-methoxyphenyl)-5-ethyl-2,3,4,5-tetrahydropyridazin-3-one;

2-(4-picolinoylaminobenzyl)-6-(3-fluoromethoxy-4-methoxyphenyl)-5-ethyl-2,3,4,5-tetrahydropyridazin-3-one;

2-(4-picolinoylaminobenzyl)-6-(3-methoxy-4-ethoxyphenyl)-5-ethyl-2,3,4,5-tetrahydropyridazin-3-one;

2-(4-picolinoylaminobenzyl)-6-(3-ethoxy-4-methoxyphenyl)-5-ethyl-2,3,4,5-tetrahydropyridazin-3-one;

2-(4-picolinoylaminobenzyl)-6-(3-hydroxy-4-methoxyphenyl)-5-ethyl-2,3,4,5-tetrahydropyridazin-3-one;

2-(4-picolinoylaminobenzyl)-6-(4-methylsulfonylphenyl)-5-ethyl-2,3,4,5-tetrahydropyridazin-3-one;

2-(4-picolinoylaminobenzyl)-6-(4-methyleneoxyphenyl)-5-ethyl-2,3,4,5-tetrahydropyridazin-3-one;

2-(4-picolinoylaminobenzyl)-6-(3-cyclopentyloxy-4-methoxyphenyl)-5-ethyl-2,3,4,5-tetrahydropyridazin-3-one;

2-(3-picolinoylaminobenzyl)-6-(3-cyclopentyloxy-4-methoxyphenyl)-5-ethyl-2,3,4,5-tetrahydropyridazin-3-one;

2-(4-picolinoylaminophenethyl)-6-(3,4-dimethoxyphenyl)-2,3,4,5-tetrahydropyridazin-3-one;

2-(4-picolinoylaminophenethyl)-6-(3,4-dimethoxyphenyl)-5-ethyl-2,3,4,5-tetrahydropyridazin-3-one;

3-(4-picolinoylaminobenzyl)-5-(3,4-dimethoxyphenyl)-3,6-dihydro-1,3,4-thiadiazin-2-one;

3-(3-picolinoylaminobenzyl)-5-(3,4-dimethoxyphenyl)-3,6-dihydro-1,3,4-thiadiazin-2-one;

3-(2-picolinoylaminobenzyl)-5-(3,4-dimethoxyphenyl)-3,6-dihydro-1,3,4-thiadiazin-2-one;

3-(4-picolinoylaminobenzyl)-5-(3,4-dimethoxyphenyl)-6-ethyl-3,6-dihydro-1,3,4-thiadiazin-2-one;

3-(3-picolinoylaminobenzyl)-5-(3,4-dimethoxyphenyl)-6-ethyl-3,6-dihydro-1,3,4-thiadiazin-2-one;

3-(2-picolinoylaminobenzyl)-5-(3,4-dimethoxyphenyl)-6-ethyl-3,6-dihydro-1,3,4-thiadiazin-2-one;

3-(4-picolinoylaminobenzyl)-5-(3-methoxy-4-trifluoromethoxyphenyl)-6-ethyl-3,6-dihydro-1,3,4-thiadiazin-2-one;

3-(4-picolinoylaminobenzyl)-5-(3-methoxy-4-difluoromethoxyphenyl)-6-ethyl-3,6-dihydro-1,3,4-thiadiazin-2-one;

3-(4-picolinoylaminobenzyl)-5-(3-methoxy-4-fluoromethoxyphenyl)-6-ethyl-3,6-dihydro-1,3,4-thiadiazin-2-one;

3-(4-picolinoylaminobenzyl)-5-(3-difluoromethoxy-4-methoxyphenyl)-6-ethyl-3,6-dihydro-1,3,4-thiadiazin-2-one;

3-(4-picolinoylaminobenzyl)-5-(3-trifluoromethoxy-4-methoxyphenyl)-6-ethyl-3,6-dihydro-1,3,4-thiadiazin-2-one;

3-(4-picolinoylaminobenzyl)-5-(3-fluoromethoxy-4-methoxyphenyl)-6-ethyl-3,6-dihydro-1,3,4-thiadiazin-2-one;

3-(4-picolinoylaminobenzyl)-5-(3-methoxy-4-ethoxyphenyl)-6-ethyl-3,6-dihydro-1,3,4-thiadiazin-2-one;

3-(4-picolinoylaminobenzyl)-5-(3-ethoxy-4-methoxyphenyl)-6-ethyl-3,6-dihydro-1,3,4-thiadiazin-2-one;

3-(4-picolinoylaminobenzyl)-5-(3-hydroxy-4-methoxyphenyl)-6-ethyl-3,6-dihydro-1,3,4-thiadiazin-2-one;

3-(4-picolinoylaminobenzyl)-5-(4-methylsulfonylphenyl)-6-ethyl-3,6-dihydro-1,3,4-thiadiazin-2-one;

3-(4-picolinoylaminobenzyl)-5-(4-methyleneoxyphenyl)-6-ethyl-3,6-dihydro-1,3,4-thiadiazin-2-one;

3-(4-picolinoylaminobenzyl)-5-(3-cyclopentyloxy-4-methoxyphenyl)-6-ethyl-3,6-dihydro-1,3,4-thiadiazin-2-one;

3-(3-picolinoylaminobenzyl)-5-(3-cyclopentyloxy-4-methoxyphenyl)-6-ethyl-3,6-dihydro-1,3,4-thiadiazin-2-one;

3-(4-picolinoylamino-phenethyl)-5-(3,4-dimethoxyphenyl)-3,6-dihydro-1,3,4-thiadiazin-2-one;

3-(4-picolinoylaminophenethyl)-5-(3,4-dimethoxyphenyl)-6-ethyl-3,6-dihydro-1,3,4-thiadiazin-2-one;

3-(4-picolinoylaminobenzyl)-5-(3,4-dimethoxyphenyl)-3,6-dihydro-1,3,4-oxadiazin-2-one;

3-(3-picolinoylaminobenzyl)-5-(3,4-dimethoxyphenyl)-3,6-dihydro-1,3,4-oxadiazin-2-one;

3-(2-picolinoylaminobenzyl)-5-(3,4-dimethoxyphenyl)-3,6-dihydro-1,3,4-oxadiazin-2-one;

3-(4-picolinoylaminobenzyl)-5-(3,4-dimethoxyphenyl)-6-ethyl-3,6-dihydro-1,3,4-oxadiazin-2-one;

3-(3-picolinoylaminobenzyl)-5-(3,4-dimethoxyphenyl)-6-ethyl-3,6-dihydro-1,3,4-oxadiazin-2-one;

3-(2-picolinoylaminobenzyl)-5-(3,4-dimethoxyphenyl)-6-ethyl-3,6-dihydro-1,3,4-oxadiazin-2-one;

3-(4-picolinoylaminobenzyl)-5-(3-methoxy-4-trifluoromethoxyphenyl)-6-ethyl-3,6-dihydro-1,3,4-oxadiazin-2-one;

3-(4-picolinoylaminobenzyl)-5-(3-methoxy-4-difluoromethoxyphenyl)-6-ethyl-3,6-dihydro-1,3,4-oxadiazin-2-one;

3-(4-picolinoylaminobenzyl)-5-(3-methoxy-4-fluoromethoxyphenyl)-6-ethyl-3,6-dihydro-1,3,4-oxadiazin-2-one;

3-(4-picolinoylaminobenzyl)-5-(3-difluoromethoxy-4-methoxyphenyl)-6-ethyl-3,6-dihydro-1,3,4-oxadiazin-2-one;

3-(4-picolinoylaminobenzyl)-5-(3-trifluoromethoxy-4-methoxyphenyl)-6-ethyl-3,6-dihydro-1,3,4-oxadiazin-2-one;

3-(4-picolinoylaminobenzyl)-5-(3-fluoromethoxy-4-methoxyphenyl)-6-ethyl-3,6-dihydro-1,3,4-oxadiazin-2-one;

3-(4-picolinoylaminobenzyl)-5-(3-methoxy-4-ethoxyphenyl)-6-ethyl-3,6-dihydro-1,3,4-oxadiazin-2-one;

3-(4-picolinoylaminobenzyl)-5-(3-ethoxy-4-methoxyphenyl)-6-ethyl-3,6-dihydro-1,3,4-oxadiazin-2-one;

3-(4-picolinoylaminobenzyl)-5-(3-hydroxy-4-methoxyphenyl)-6-ethyl-3,6-dihydro-1,3,4-oxadiazin-2-one;

3-(4-picolinoylaminobenzyl)-5-(4-methylsulfonylphenyl)-6-ethyl-3,6-dihydro-1,3,4-oxadiazin-2-one;

3-(4-picolinoylaminobenzyl)-5-(4-methyleneoxyphenyl)-6-ethyl-3,6-dihydro-1,3,4-oxadiazin-2-one;

3-(4-picolinoylaminobenzyl)-5-(3-cyclopentyloxy-4-methoxyphenyl)-6-ethyl-3,6-dihydro-1,3,4-oxadiazin-2-one;

3-(3-picolinoylaminobenzyl)-5-(3-cyclopentyloxy-4-methoxyphenyl)-6-ethyl-3,6-dihydro-1,3,4-oxadiazin-2-one;

3-(4-picolinoylaminophenethyl)-5-(3,4-dimethoxyphenyl)-3,6-dihydro-1,3,4-oxadiazin-2-one;

3-(4-picolinoylaminophenethyl)-5-(3,4-dimethoxyphenyl)-6-ethyl-3,6-dihydro-1,3,4-oxadiazin-2-one;

2-(4-(furan-2-carbonylamino)benzyl)-6-(3,4-dimethoxyphenyl)-2,3,4,5-tetrahydropyridazin-3-one;

2-(3-(furan-2-carbonylamino)benzyl)-6-(3,4-dimethoxyphenyl)-2,3,4,5-tetrahydropyridazin-3-one;

2-(2-(furan-2-carbonylamino)benzyl)-6-(3,4-dimethoxyphenyl)-2,3,4,5-tetrahydropyridazin-3-one;

2-(4-(furan-2-carbonylamino)benzyl)-6-(3,4-dimethoxyphenyl)-5-ethyl-2,3,4,5-tetrahydropyridazin-3-one;

2-(3-(furan-2-carbonylamino)benzyl)-6-(3,4-dimethoxyphenyl)-5-ethyl-2,3,4,5-tetrahydropyridazin-3-one;

2-(2-(furan-2-carbonylamino)benzyl)-6-(3,4-dimethoxyphenyl)-5-ethyl-2,3,4,5-tetrahydropyridazin-3-one;

2-(4-(furan-2-carbonylamino)benzyl)-6-(3-methoxy-4-trifluoromethoxyphenyl)-5-ethyl-2,3,4,5-tetrahydropyridazin-3-one;

2-(4-(furan-2-carbonylamino)benzyl)-6-(3-methoxy-4-difluoromethoxyphenyl)-5-ethyl-2,3,4,5-tetrahydropyridazin-3-one;

2-(4-(furan-2-carbonylamino)benzyl)-6-(3-methoxy-4-fluoromethoxyphenyl)-5-ethyl-2,3,4,5-tetrahydropyridazin-3-one;

2-(4-(furan-2-carbonylamino)benzyl)-6-(3-difluoromethoxy-4-methoxyphenyl)-5-ethyl-2,3,4,5-tetrahydropyridazin-3-one;

2-(4-(furan-2-carbonylamino)benzyl)-6-(3-trifluoromethoxy-4-methoxyphenyl)-5-ethyl-2,3,4,5-tetrahydropyridazin-3-one;

2-(4-(furan-2-carbonylamino)benzyl)-6-(3-fluoromethoxy-4-methoxyphenyl)-5-ethyl-2,3,4,5-tetrahydropyridazin-3-one;

2-(4-(furan-2-carbonylamino)benzyl)-6-(3-methoxy-4-ethoxyphenyl)-5-ethyl-2,3,4,5-tetrahydropyridazin-3-one;

2-(4-(furan-2-carbonylamino)benzyl)-6-(3-ethoxy-4-methoxyphenyl)-5-ethyl-2,3,4,5-tetrahydropyridazin-3-one;

2-(4-(furan-2-carbonylamino)benzyl)-6-(3-hydroxy-4-methoxyphenyl)-5-ethyl-2,3,4,5-tetrahydropyridazin-3-one;

2-(4-(furan-2-carbonylamino)benzyl)-6-(4-methylsulfonylphenyl)-5-ethyl-2,3,4,5-tetrahydropyridazin-3-one;

2-(4-(furan-2-carbonylamino)benzyl)-6-(4-methyleneoxyphenyl)-5-ethyl-2,3,4,5-tetrahydropyridazin-3-one;

2-(4-(furan-2-carbonylamino)benzyl)-6-(3-cyclopentyloxy-4-methoxyphenyl)-5-ethyl-2,3,4,5-tetrahydropyridazin-3-one;

2-(3-(furan-2-carbonylamino)benzyl)-6-(3-cyclopentyloxy-4-methoxyphenyl)-5-ethyl-2,3,4,5-tetrahydropyridazin-3-one;

2-(4-(furan-2-carbonylamino)phenethyl)-6-(3,4-dimethoxyphenyl)-2,3,4,5-tetrahydropyridazin-3-one;

2-(4-(furan-2-carbonylamino)phenethyl)-6-(3,4-dimethoxyphenyl)-5-ethyl-2,3,4,5-tetrahydropyridazin-3-one;

3-(4-(furan-2-carbonylamino)benzyl)-5-(3,4-dimethoxyphenyl)-3,6-dihydro-1,3,4-thiadiazin-2-one;

3-(3-(furan-2-carbonylamino)benzyl)-5-(3,4-dimethoxyphenyl)-3,6-dihydro-1,3,4-thiadiazin-2-one;

3-(2-(furan-2-carbonylamino)benzyl)-5-(3,4-dimethoxyphenyl)-3,6-dihydro-1,3,4-thiadiazin-2-one;

3-(4-(furan-2-carbonylamino)benzyl)-5-(3,4-dimethoxyphenyl)-6-ethyl-3,6-dihydro-1,3,4-thiadiazin-2-one;

3-(3-(furan-2-carbonylamino)benzyl)-5-(3,4-dimethoxyphenyl)-6-ethyl-3,6-dihydro-1,3,4-thiadiazin-2-one;

3-(2-(furan-2-carbonylamino)benzyl)-5-(3,4-dimethoxyphenyl)-6-ethyl-3,6-dihydro-1,3,4-thiadiazin-2-one;

3-(4-(furan-2-carbonylamino)benzyl)-5-(3-methoxy-4-trifluoromethoxyphenyl)-6-ethyl-3,6-dihydro-1,3,4-thiadiazin-2-one;

3-(4-(furan-2-carbonylamino)benzyl)-5-(3-methoxy-4-difluoromethoxyphenyl)-6-ethyl-3,6-dihydro-1,3,4-thiadiazin-2-one;

3-(4-(furan-2-carbonylamino)benzyl)-5-(3-methoxy-4-fluoromethoxyphenyl)-6-ethyl-3,6-dihydro-1,3,4-thiadiazin-2-one;

3-(4-(furan-2-carbonylamino)benzyl)-5-(3-difluoromethoxy-4-methoxyphenyl)-6-ethyl-3,6-dihydro-1,3,4-thiadiazin-2-one;

3-(4-(furan-2-carbonylamino)benzyl)-5-(3-trifluoromethoxy-4-methoxyphenyl)-6-ethyl-3,6-dihydro-1,3,4-thiadiazin-2-one;

3-(4-(furan-2-carbonylamino)benzyl)-5-(3-fluoromethoxy-4-methoxyphenyl)-6-ethyl-3,6-dihydro-1,3,4-thiadiazin-2-one;

3-(4-(furan-2-carbonylamino)benzyl)-5-(3-methoxy-4-ethoxyphenyl)-6-ethyl-3,6-dihydro-1,3,4-thiadiazin-2-one;

3-(4-(furan-2-carbonylamino)benzyl)-5-(3-ethoxy-4-methoxyphenyl)-6-ethyl-3,6-dihydro-1,3,4-thiadiazin-2-one;

3-(4-(furan-2-carbonylamino)benzyl)-5-(3-hydroxy-4-methoxyphenyl)-6-ethyl-3,6-dihydro-1,3,4-thiadiazin-2-one;

3-(4-(furan-2-carbonylamino)benzyl)-5-(4-methylsulfonylphenyl)-6-ethyl-3,6-dihydro-1,3,4-thiadiazin-2-one;

3-(4-(furan-2-carbonylamino)benzyl)-5-(4-methyleneoxyphenyl)-6-ethyl-3,6-dihydro-1,3,4-thiadiazin-2-one;

3-(4-(furan-2-carbonylamino)benzyl)-5-(3-cyciopentyloxy-4-methoxyphenyl)-6-ethyl-3,6-dihydro-1,3,4-thiadiazin-2-one;

3-(3-(furan-2-carbonylamino)benzyl)-5-(3-cyclopentyloxy-4-methoxyphenyl)-6-ethyl-3,6-dihydro-1,3,4-thiadiazin-2-one;

3-(4-(furan-2-carbonylamino)phenethyl)-5-(3,4-dimethoxyphenyl)-3,6-dihydro-1,3,4-thiadiazin-2-one;

3-(4-(furan-2-carbonylamino)phenethyl)-5-(3,4-dimethoxyphenyl)-6-ethyl-3,6-dihydro-1,3,4-thiadiazin-2-one;

3-(4-(furan-2-carbonylamino)benzyl)-5-(3,4-dimethoxyphenyl)-3,6-dihydro-1,3,4-oxadiazin-2-one;

3-(3-(furan-2-carbonylamino)benzyl)-5-(3,4-dimethoxyphenyl)-3,6-dihydro-1,3,4-oxadiazin-2-one;

3-(2-(furan-2-carbonylamino)benzyl)-5-(3,4-dimethoxyphenyl)-3,6-dihydro-1,3,4-oxadiazin-2-one;

3-(4-(furan-2-carbonylamino)benzyl)-5-(3,4-dimethoxyphenyl)-6-ethyl-3,6-dihydro-1,3,4-oxadiazin-2-one;

3-(3-(furan-2-carbonylamino)benzyl)-5-(3,4-dimethoxyphenyl)-6-ethyl-3,6-dihydro-1,3,4-oxadiazin-2-one;

3-(2-(furan-2-carbonylamino)benzyl)-5-(3,4-dimethoxyphenyl)-6-ethyl-3,6-dihydro-1,3,4-oxadiazin-2-one;

3-(4-(furan-2-carbonylamino)benzyl)-5-(3-methoxy-4-trifluoromethoxyphenyl)-6-ethyl-3,6-dihydro-1,3,4-oxadiazin-2-one;

3-(4-(furan-2-carbonylamino)benzyl)-5-(3-methoxy-4-difluoromethoxyphenyl)-6-ethyl-3,6-dihydro-1,3,4-oxadiazin-2-one;

3-(4-(furan-2-carbonylamino)benzyl)-5-(3-methoxy-4-fluoromethoxyphenyl)-6-ethyl-3,6-dihydro-1,3,4-oxadiazin-2-one;

3-(4-(furan-2-carbonylamino)benzyl)-5-(3-difluoromethoxy-4-methoxyphenyl)-6-ethyl-3,6-dihydro-1,3,4-oxadiazin-2-one;

3-(4-(furan-2-carbonylamino)benzyl)-5-(3-trifluoromethoxy-4-methoxyphenyl)-6-ethyl-3,6-dihydro-1,3,4-oxadiazin-2-one;

3-(4-(furan-2-carbonylamino)benzyl)-5-(3-fluoromethoxy-4-methoxyphenyl)-6-ethyl-3,6-dihydro-1,3,4-oxadiazin-2-one;

3-(4-(furan-2-carbonylamino)benzyl)-5-(3-methoxy-4-ethoxyphenyl)-6-ethyl-3,6-dihydro-1,3,4-oxadiazin-2-one;

3-(4-(furan-2-carbonylamino)benzyl)-5-(3-ethoxy-4-methoxyphenyl)-6-ethyl-3,6-dihydro-1,3,4-oxadiazin-2-one;

3-(4-(furan-2-carbonylamino)benzyl)-5-(3-hydroxy-4-methoxyphenyl)-6-ethyl-3,6-dihydro-1,3,4-oxadiazin-2-one;

3-(4-(furan-2-carbonylamino)benzyl)-5-(4-methylsulfonylphenyl)-6-ethyl-3,6-dihydro-1,3,4-oxadiazin-2-one;

3-(4-(furan-2-carbonylamino)benzyl)-5-(4-methyleneoxyphenyl)-6-ethyl-3,6-dihydro-1,3,4-oxadiazin-2-one;

3-(4-(furan-2-carbonylamino)benzyl)-5-(3-cyclopentyloxy-4-methoxyphenyl)-6-ethyl-3,6-dihydro-1,3,4-oxadiazin-2-one;

3-(3-(furan-2-carbonylamino)benzyl)-5-(3-cyclopentyloxy-4-methoxyphenyl)-6-ethyl-3,6-dihydro-1,3,4-oxadiazin-2-one;

3-(4-(furan-2-carbonylamino)phenethyl)-5-(3,4-dimethoxyphenyl)-3,6-dihydro-1,3,4-oxadiazin-2-one;

3-(4-(furan-2-carbonylamino)phenethyl)-5-(3,4-dimethoxyphenyl)-6-ethyl-3,6-dihydro-1,3,4-oxadiazin-2-one;

2-(4-(pyrrole-2-carbonylamino)benzyl)-6-(3,4-dimethoxyphenyl)-2,3,4,5-tetrahydropyridazin-3-one;

2-(3-(pyrrole-2-carbonylamino)benzyl)-6-(3,4-dimethoxyphenyl)-2,3,4,5-tetrahydropyridazin-3-one;

2-(2-(pyrrole-2-carbonylamino)benzyl)-6-(3,4-dimethoxyphenyl)-2,3,4,5-tetrahydropyridazin-3-one;

2-(4-(pyrrole-2-carbonylamino)benzyl)-6-(3,4-dimethoxyphenyl)-5-ethyl-2,3,4,5-tetrahydropyridazin-3-one;

2-(3-(pyrrole-2-carbonylamino)benzyl)-6-(3,4-dimethoxyphenyl)-5-ethyl-2,3,4,5-tetrahydropyridazin-3-one;

2-(2-(pyrrole-2-carbonylamino)benzyl)-6-(3,4-dimethoxyphenyl)-5-ethyl-2,3,4,5-tetrahydropyridazin-3-one;

2-(4-(pyrrole-2-carbonylamino)benzyl)-6-(3-methoxy-4-trifluoromethoxyphenyl)-5-ethyl-2,3,4,5-tetrahydropyridazin-3-one;

2-(4-(pyrrole-2-carbonylamino)benzyl)-6-(3-methoxy-4-difluoromethoxyphenyl)-5-ethyl-2,3,4,5-tetrahydropyridazin-3-one;

2-(4-(pyrrole-2-carbonylamino)benzyl)-6-(3-methoxy-4-fluoromethoxyphenyl)-5-ethyl-2,3,4,5-tetrahydropyridazin-3-one;

2-(4-(pyrrole-2-carbonylamino)benzyl)-6-(3-difluoromethoxy-4-methoxyphenyl)-5-ethyl-2,3,4,5-tetrahydropyridazin-3-one;

2-(4-(pyrrole-2-carbonylamino)benzyl)-6-(3-trifluoromethoxy-4-methoxyphenyl)-5-ethyl-2,3,4,5-tetrahydropyridazin-3-one;

2-(4-(pyrrole-2-carbonylamino)benzyl)-6-(3-fluoromethoxy-4-methoxyphenyl)-5-ethyl-2,3,4,5-tetrahydropyridazin-3-one;

2-(4-(pyrrole-2-carbonylamino)benzyl)-6-(3-methoxy-4-ethoxyphenyl)-5-ethyl-2,3,4,5-tetrahydropyridazin-3-one;

2-(4-(pyrrole-2-carbonylamino)benzyl)-6-(3-ethoxy-4-methoxyphenyl)-5-ethyl-2,3,4,5-tetrahydropyridazin-3-one;

2-(4-(pyrrole-2-carbonylamino)benzyl)-6-(3-hydroxy-4-methoxyphenyl)-5-ethyl-2,3,4,5-tetrahydropyridazin-3-one;

2-(4-(pyrrole-2-carbonylamino)benzyl)-6-(4-methylsulfonylphenyl)-5-ethyl-2,3,4,5-tetrahydropyridazin-3-one;

2-(4-(pyrrole-2-carbonylamino)benzyl)-6-(4-methyleneoxyphenyl)-5-ethyl-2,3,4,5-tetrahydropyridazin-3-one;

2-(4-(pyrrole-2-carbonylamino)benzyl)-6-(3-cyclopentyloxy-4-methoxyphenyl)-5-ethyl-2,3,4,5-tetrahydropyridazin-3-one;

2-(3-(pyrrole-2-carbonylamino)benzyl)-6-(3-cyclopentyloxy-4-methoxyphenyl)-5-ethyl-2,3,4,5-tetrahydropyridazin-3-one;

2-(4-(pyrrole-2-carbonylamino)phenethyl)-6-(3,4-dimethoxyphenyl)-2,3,4,5-tetrahydropyridazin-3-one;

2-(4-(pyrrole-2-carbonylamino)phenethyl)-6-(3,4-dimethoxyphenyl)-5-ethyl-2,3,4,5-tetrahydropyridazin-3-one;

3-(4-(pyrrole-2-carbonylamino)benzyl)-5-(3,4-dimethoxyphenyl)-3,6-dihydro-1,3,4-thiadiazin-2-one;

3-(3-(pyrrole-2-carbonylamino)benzyl)-5-(3,4-dimethoxyphenyl)-3,6-dihydro-1,3,4-thiadiazin-2-one;

3-(2-(pyrrole-2-carbonylamino)benzyl)-5-(3,4-dimethoxyphenyl)-3,6-dihydro-1,3,4-thiadiazin-2-one;

3-(4-(pyrrole-2-carbonylamino)benzyl)-5-(3,4-dimethoxyphenyl)-6-ethyl-3,6-dihydro-1,3,4-thiadiazin-2-one;

3-(3-(pyrrole-2-carbonylamino)benzyl)-5-(3,4-dimethoxyphenyl)-6-ethyl-3,6-dihydro-1,3,4-thiadiazin-2-one;

3-(2-(pyrrole-2-carbonylamino)benzyl)-5-(3,4-dimethoxyphenyl)-6-ethyl-3,6-dihydro-1,3,4-thiadiazin-2-one;

3-(4-(pyrrole-2-carbonylamino)benzyl)-5-(3-methoxy-4-trifluoromethoxyphenyl)-6-ethyl-3,6-dihydro-1,3,4-thiadiazin-2-one;

3-(4-(pyrrole-2-carbonylamino)benzyl)-5-(3-methoxy-4-difluoromethoxyphenyl)-6-ethyl-3,6-dihydro-1,3,4-thiadiazin-2-one;

3-(4-(pyrrole-2-carbonylamino)benzyl)-5-(3-methoxy-4-fluoromethoxyphenyl)-6-ethyl-3,6-dihydro-1,3,4-thiadiazin-2-one;

3-(4-(pyrrole-2-carbonylamino)benzyl)-5-(3-difluoromethoxy-4-methoxyphenyl)-6-ethyl-3,6-dihydro-1,3,4-thiadiazin-2-one;

3-(4-(pyrrole-2-carbonylamino)benzyl)-5-(3-trifluoromethoxy-4-methoxyphenyl)-6-ethyl-3,6-dihydro-1,3,4-thiadiazin-2-one;

3-(4-(pyrrole-2-carbonylamino)benzyl)-5-(3-fluoromethoxy-4-methoxyphenyl)-6-ethyl-3,6-dihydro-1,3,4-thiadiazin-2-one;

3-(4-(pyrrole-2-carbonylamino)benzyl)-5-(3-methoxy-4-ethoxyphenyl)-6-ethyl-3,6-dihydro-1,3,4-thiadiazin-2-one;

3-(4-(pyrrole-2-carbonylamino)benzyl)-5-(3-ethoxy-4-methoxyphenyl)-6-ethyl-3,6-dihydro-1,3,4-thiadiazin-2-one;

3-(4-(pyrrole-2-carbonylamino)benzyl)-5-(3-hydroxy-4-methoxyphenyl)-6-ethyl-3,6-dihydro-1,3,4-thiadiazin-2-one;

3-(4-(pyrrole-2-carbonylamino)benzyl)-5-(4-methylsulfonylphenyl)-6-ethyl-3,6-dihydro-1,3,4-thiadiazin-2-one;

3-(4-( pyrrole-2-carbonylamino)benzyl)-5-(4-methyleneoxyphenyl)-6-ethyl-3,6-dihydro-1,3,4-thiadiazin-2-one;

3-(4-(pyrrole-2-carbonylamino)benzyl)-5-(3-cyclopentyloxy-4-methoxyphenyl)-6-ethyl-3,6-dihydro-1,3,4-thiadiazin-2-one;

3-(3-(pyrrole-2-carbonylamino)benzyl)-5-(3-cyclopentyloxy-4-methoxyphenyl)-6-ethyl-3,6-dihydro-1,3,4-thiadiazin-2-one;

3-(4-(pyrrole-2-carbonylamino)phenethyl)-5-(3,4-dimethoxyphenyl)-3,6-dihydro-1,3,4-thiadiazin-2-one;

3-(4-(pyrrole-2-carbonylamino)phenethyl)-5-(3,4-dimethoxyphenyl)-6-ethyl-3,6-dihydro-1,3,4-thiadiazin-2-one;

3-(4-(pyrrole-2-carbonylamino)benzyl)-5-(3,4-dimethoxyphenyl)-3,6-dihydro-1,3,4-oxadiazin-2-one;

3-(3-(pyrrole-2-carbonylamino)benzyl)-5-(3,4-dimethoxyphenyl)-3,6-dihydro-1,3,4-oxadiazin-2-one;

3-(2-(pyrrole-2-carbonylamino)benzyl)-5-(3,4-dimethoxyphenyl)-3,6-dihydro-1,3,4-oxadiazin-2-one;

3-(4-(pyrrole-2-carbonylamino)benzyl)-5-(3,4-dimethoxyphenyl)-6-ethyl-3,6-dihydro-1,3,4-oxadiazin-2-one;

3-(3-(pyrrole-2-carbonylamino)benzyl)-5-(3,4-dimethoxyphenyl)-6-ethyl-3,6-dihydro-1,3,4-oxadiazin-2-one;

3-(2-(pyrrole-2-carbonylamino)benzyl)-5-(3,4-dimethoxyphenyl)-6-ethyl-3,6-dihydro-1,3,4-oxadiazin-2-one;

3-(4-(pyrrole-2-carbonylamino)benzyl)-5-(3-methoxy-4-trifluoromethoxyphenyl)-6-ethyl-3,6-dihydro-1,3,4-oxadiazin-2-one;

3-(4-(pyrrole-2-carbonylamino)benzyl)-5-(3-methoxy-4-difluoromethoxyphenyl)-6-ethyl-3,6-dihydro-1,3,4-oxadiazin-2-one;

3-(4-(pyrrole-2-carbonylamino)benzyl)-5-(3-methoxy-4-fluoromethoxyphenyl)-6-ethyl-3,6-dihydro-1,3,4-oxadiazin-2-one;

3-(4-(pyrrole-2-carbonylamino)benzyl)-5-(3-difluoromethoxy-4-methoxyphenyl)-6-ethyl-3,6-dihydro-1,3,4-oxadiazin-2-one;

3-(4-(pyrrole-2-carbonylamino)benzyl)-5-(3-trifluoromethoxy-4-methoxyphenyl)-6-ethyl-3,6-dihydro-1,3,4-oxadiazin-2-one;

3-(4-(pyrrole-2-carbonylamino)benzyl)-5-(3-fluoromethoxy-4-methoxyphenyl)-6-ethyl-3,6-dihydro-1,3,4-oxadiazin-2-one;

3-(4-(pyrrole-2-carbonylamino)benzyl)-5-(3-methoxy-4-ethoxyphenyl)-6-ethyl-3,6-dihydro-1,3,4-oxadiazin-2-one;

3-(4-(pyrrole-2-carbonylamino)benzyl)-5-(3-ethoxy-4-methoxyphenyl)-6-ethyl-3,6-dihydro-1,3,4-oxadiazin-2-one;

3-(4-(pyrrole-2-carbonylamino)benzyl)-5-(3-hydroxy-4-methoxyphenyl)-6-ethyl-3,6-dihydro-1,3,4-oxadiazin-2-one;

3-(4-(pyrrole-2-carbonylamino)benzyl)-5-(4-methylsulfonylphenyl)-6-ethyl-3,6-dihydro-1,3,4-oxadiazin-2-one;

3-(4-(pyrrole-2-carbonylamino)benzyl)-5-(4-methyleneoxyphenyl)-6-ethyl-3,6-dihydro-1,3,4-oxadiazin-2-one;

3-(4-(pyrrole-2-carbonylamino)benzyl)-5-(3-cyclopentyloxy-4-methoxyphenyl)-6-ethyl-3,6-dihydro-1,3,4-oxadiazin-2-one;

3-(3-(pyrrole-2-carbonylamino)benzyl)-5-(3-cyclopentyloxy-4-methoxyphenyl)-6-ethyl-3,6-dihydro-1,3,4-oxadiazin-2-one;

3-(4-(pyrrole-2-carbonylamino)phenethyl)-5-(3,4-dimethoxyphenyl)-3,6-dihydro-1,3,4-oxadiazin-2-one;

3-(4-(pyrrole-2-carbonylamino)phenethyl)-5-(3,4-dimethoxyphenyl)-6-ethyl-3,6-dihydro-1,3,4-oxadiazin-2-one;

2-(4-(thiophene-2-carbonylamino)benzyl)-6-(3,4-dimethoxyphenyl)-2,3,4,5-tetrahydropyridazin-3-one;

2-(3-(thiophene-2-carbonylamino)benzyl)-6-(3,4-dimethoxyphenyl)-2,3,4,5-tetrahydropyridazin-3-one;

2-(2-(thiophene-2-carbonylamino)benzyl)-6-(3,4-dimethoxyphenyl)-2,3,4,5-tetrahydropyridazin-3-one;

2-(4-(thiophene-2-carbonylamino)benzyl)-6-(3,4-dimethoxyphenyl)-5-ethyl-2,3,4,5-tetrahydropyridazin-3-one;

2-(3-(thiophene-2-carbonylamino)benzyl)-6-(3,4-dimethoxyphenyl)-5-ethyl-2,3,4,5-tetrahydropyridazin-3-one;

2-(2-(thiophene-2-carbonylamino)benzyl)-6-(3,4-dimethoxyphenyl)-5-ethyl-2,3,4,5-tetrahydropyridazin-3-one;

2-(4-(thiophene-2-carbonylamino)benzyl)-6-(3-methoxy-4-trifluoromethoxyphenyl)-5-ethyl-2,3,4,5-tetrahydropyridazin-3-one;

2-(4-(thiophene-2-carbonylamino)benzyl)-6-(3-methoxy-4-difluoromethoxyphenyl)-5-ethyl-2,3,4,5-tetrahydropyridazin-3-one;

2-(4-(thiophene-2-carbonylamino)benzyl)-6-(3-methoxy-4-fluoromethoxyphenyl)-5-ethyl-2,3,4,5-tetrahydropyridazin-3-one;

2-(4-(thiophene-2-carbonylamino)benzyl)-6-(3-difluoromethoxy-4-methoxyphenyl)-5-ethyl-2,3,4,5-tetrahydropyridazin-3-one;

2-(4-(thiophene-2-carbonylamino)benzyl)-6-(3-trifluoromethoxy-4-methoxyphenyl)-5-ethyl-2,3,4,5-tetrahydropyridazin-3-one;

2-(4-(thiophene-2-carbonylamino)benzyl)-6-(3-fluoromethoxy-4-methoxyphenyl)-5-ethyl-2,3,4,5-tetrahydropyridazin-3-one;

2-(4-(thiophene-2-carbonylamino)benzyl)-6-(3-methoxy-4-ethoxyphenyl)-5-ethyl-2,3,4,5-tetrahydropyridazin-3-one;

2-(4-(thiophene-2-carbonylamino)benzyl)-6-(3-ethoxy-4-methoxyphenyl)-5-ethyl-2,3,4,5-tetrahydropyridazin-3-one;

2-(4-(thiophene-2-carbonylamino)benzyl)-6-(3-hydroxy-4-methoxyphenyl)-5-ethyl-2,3,4,5-tetrahydropyridazin-3-one;

2-(4-(thiophene-2-carbonylamino)benzyl)-6-(4-methylsulfonylphenyl)-5-ethyl-2,3,4,5-tetrahydropyridazin-3-one;

2-(4-(thiophene-2-carbonylamino)benzyl)-6-(4-methyleneoxyphenyl)-5-ethyl-2,3,4,5-tetrahydropyridazin-3-one;

2-(4-(thiophene-2-carbonylamino)benzyl)-6-(3-cyclopentyloxy-4-methoxyphenyl)-5-ethyl-2,3,4,5-tetrahydropyridazin-3-one;

2-(3-(thiophene-2-carbonylamino)benzyl)-6-(3-cyclopentyloxy-4-methoxyphenyl)-5-ethyl-2,3,4,5-tetrahydropyridazin-3-one;

2-(4-(thiophene-2-carbonylamino)phenethyl)-6-(3,4-dimethoxyphenyl)-2,3,4,5-tetrahydropyridazin-3-one;

2-(4-(thiophene-2-carbonylamino)phenethyl)-6-(3,4-dimethoxyphenyl)-5-ethyl-2,3,4,5-tetrahydropyridazin-3-one;

3-(4-(thiophene-2-carbonylamino)benzyl)-5-(3,4-dimethoxyphenyl)-3,6-dihydro-1,3,4-thiadiazin-2-one, m.p. 186°;

3-(3-(thiophene-2-carbonylamino)benzyl)-5-(3,4-dimethoxyphenyl)-3,6-dihydro-1,3,4-thiadiazin-2-one;

3-(2-(thiophene-2-carbonylamino)benzyl)-5-(3,4-dimethoxyphenyl)-3,6-dihydro-1,3,4-thiadiazin-2-one;

3-(4-(thiophene-2-carbonylamino)benzyl)-5-(3,4-dimethoxyphenyl)-6-ethyl-3,6-dihydro-1,3,4-thiadiazin-2-one;

3-(3-(thiophene-2-carbonylamino)benzyl)-5-(3,4-dimethoxyphenyl)-6-ethyl-3,6-dihydro-1,3,4-thiadiazin-2-one;

3-(2-(thiophene-2-carbonylamino)benzyl)-5-(3,4-dimethoxyphenyl)-6-ethyl-3,6-dihydro-1,3,4-thiadiazin-2-one;

3-(4-(thiophene-2-carbonylamino)benzyl)-5-(3-methoxy-4-trifluoromethoxyphenyl)-6-ethyl-3,6-dihydro-1,3,4-thiadiazin-2-one;

3-(4-(thiophene-2-carbonylamino)benzyl)-5-(3-methoxy-4-difluoromethoxyphenyl)-6-ethyl-3,6-dihydro-1,3,4-thiadiazin-2-one;

3-(4-(thiophene-2-carbonylamino)benzyl)-5-(3-methoxy-4-fluoromethoxyphenyl)-6-ethyl-3,6-dihydro-1,3,4-thiadiazin-2-one;

3-(4-(thiophene-2-carbonylamino)benzyl)-5-(3-difluoromethoxy-4-methoxyphenyl)-6-ethyl-3,6-dihydro-1,3,4-thiadiazin-2-one;

3-(4-(thiophene-2-carbonylamino)benzyl)-5-(3-trifluoromethoxy-4-methoxyphenyl)-6-ethyl-3,6-dihydro-1,3,4-thiadiazin-2-one;

3-(4-(thiophene-2-carbonylamino)benzyl)-5-(3-fluoromethoxy-4-methoxyphenyl)-6-ethyl-3,6-dihydro-1,3,4-thiadiazin-2-one;

3-(4-(thiophene-2-carbonylamino)benzyl)-5-(3-methoxy-4-ethoxyphenyl)-6-ethyl-3,6-dihydro-1,3,4-thiadiazin-2-one;

3-(4-(thiophene-2-carbonylamino)benzyl)-5-(3-ethoxy-4-methoxyphenyl)-6-ethyl-3,6-dihydro-1,3,4-thiadiazin-2-one;

3-(4-(thiophene-2-carbonylamino)benzyl)-5-(3-hydroxy-4-methoxyphenyl)-6-ethyl-3,6-dihydro-1,3,4-thiadiazin-2-one;

3-(4-(thiophene-2-carbonylamino)benzyl)-5-(4-methylsulfonylphenyl)-6-ethyl-3,6-dihydro-1,3,4-thiadiazin-2-one;

3-(4-(thiophene-2-carbonylamino)benzyl)-5-(4-methyleneoxyphenyl)-6-ethyl-3,6-dihydro-1,3,4-thiadiazin-2-one;

3-(4-(thiophene-2-carbonylamino)benzyl)-5-(3-cyclopentyloxy-4-methoxyphenyl)-6-ethyl-3,6-dihydro-1,3,4-thiadiazin-2-one;

3-(3-(thiophene-2-carbonylamino)benzyl)-5-(3-cyclopentyloxy-4-methoxyphenyl)-6-ethyl-3,6-dihydro-1,3,4-thiadiazin-2-one;

3-(4-(thiophene-2-carbonylamino)phenethyl)-5-(3,4-dimethoxyphenyl)-3,6-dihydro-1,3,4-thiadiazin-2-one;

3-(4-(thiophene-2-carbonylamino)phenethyl)-5-(3,4-dimethoxyphenyl)-6-ethyl-3,6-dihydro-1,3,4-thiadiazin-2-one;

3-(4-(thiophene-2-carbonylamino)benzyl)-5-(3,4-dimethoxyphenyl)-3,6-dihydro-1,3,4-oxadiazin-2-one;

3-(3-(thiophene-2-carbonylamino)benzyl)-5-(3,4-dimethoxyphenyl)-3,6-dihydro-1,3,4-oxadiazin-2-one;

3-(2-(thiophene-2-carbonylamino)benzyl)-5-(3,4-dimethoxyphenyl)-3,6-dihydro-1,3,4-oxadiazin-2-one;

3-(4-(thiophene-2-carbonylamino)benzyl)-5-(3,4-dimethoxyphenyl)-6-ethyl-3,6-dihydro-1,3,4-oxadiazin-2-one;

3-(3-(thiophene-2-carbonylamino)benzyl)-5-(3,4-dimethoxyphenyl)-6-ethyl-3,6-dihydro-1,3,4-oxadiazin-2-one;

3-(2-(thiophene-2-carbonylamino)benzyl)-5-(3,4-dimethoxyphenyl)-6-ethyl-3,6-dihydro-1,3,4-oxadiazin-2-one;

3-(4-(thiophene-2-carbonylamino)benzyl)-5-(3-methoxy-4-trifluoromethoxyphenyl)-6-ethyl-3,6-dihydro-1,3,4-oxadiazin-2-one;

3-(4-(thiophene-2-carbonylamino)benzyl)-5-(3-methoxy-4-difluoromethoxyphenyl)-6-ethyl-3,6-dihydro-1,3,4-oxadiazin-2-one;

3-(4-(thiophene-2-carbonylamino)benzyl)-5-(3-methoxy-4-fluoromethoxyphenyl)-6-ethyl-3,6-dihydro-1,3,4-oxadiazin-2-one;

3-(4-(thiophene-2-carbonylamino)benzyl)-5-(3-difluoromethoxy-4-methoxyphenyl)-6-ethyl-3,6-dihydro-1,3,4-oxadiazin-2-one;

3-(4-(thiophene-2-carbonylamino)benzyl)-5-(3-trifluoromethoxy-4-methoxyphenyl)-6-ethyl-3,6-dihydro-1,3,4-oxadiazin-2-one;

3-(4-(thiophene-2-carbonylamino)benzyl)-5-(3-fluoromethoxy-4-methoxyphenyl)-6-ethyl-3,6-dihydro-1,3,4-oxadiazin-2-one;

3-(4-(thiophene-2-carbonylamino)benzyl)-5-(3-methoxy-4-ethoxyphenyl)-6-ethyl-3,6-dihydro-1,3,4-oxadiazin-2-one;

3-(4-(thiophene-2-carbonylamino)benzyl)-5-(3-ethoxy-4-methoxyphenyl)-6-ethyl-3,6-dihydro-1,3,4-oxadiazin-2-one;

3-(4-(thiophene-2-carbonylamino)benzyl)-5-(3-hydroxy-4-methoxyphenyl)-6-ethyl-3,6-dihydro-1,3,4-oxadiazin-2-one;

3-(4-(thiophene-2-carbonylamino)benzyl)-5-(4-methylsulfonylphenyl)-6-ethyl-3,6-dihydro-1,3,4-oxadiazin-2-one;

3-(4-(thiophene-2-carbonylamino)benzyl)-5-(4-methyleneoxyphenyl)-6-ethyl-3,6-dihydro-1,3,4-oxadiazin-2-one;

3-(4-(thiophene-2-carbonylamino)benzyl)-5-(3-cyclopentyloxy-4-methoxyphenyl)-6-ethyl-3,6-dihydro-1,3,4-oxadiazin-2-one;

3-(3-(thiophene-2-carbonylamino)benzyl)-5-(3-cyclopentyloxy-4-methoxyphenyl)-6-ethyl-3,6-dihydro-1,3,4-oxadiazin-2-one;

3-(4-(thiophene-2-carbonylamino)phenethyl)-5-(3,4-dimethoxyphenyl)-3,6-dihydro-1,3,4-oxadiazin-2-one;

3-(4-(thiophene-2-carbonylamino)phenethyl)-5-(3,4-dimethoxyphenyl)-6-ethyl-3,6-dihydro-1,3,4-oxadiazin-2-one;

2-(4-pyrazinecarbonylaminobenzyl)-6-(3,4-dimethoxyphenyl)-2,3,4,5-tetrahydropyridazin-3-one, m.p. 197°;

2-(3-pyrazinecarbonylaminobenzyl)-6-(3,4-dimethoxyphenyl)-2,3,4,5-tetrahydropyridazin-3-one;

2-(2-pyrazinecarbonylaminobenzyl)-6-(3,4-dimethoxyphenyl)-2,3,4,5-tetrahydropyridazin-3-one;

2-(4-pyrazinecarbonylaminobenzyl)-6-(3,4-dimethoxyphenyl)-5-ethyl-2,3,4,5-tetrahydropyridazin-3-one;

2-(3-pyrazinecarbonylaminobenzyi)-6-(3,4-dimethoxyphenyl)-5-ethyl-2,3,4,5-tetrahydropyridazin-3-one;

2-(2-pyrazinecarbonylaminobenzyl)-6-(3,4-dimethoxyphenyl)-5-ethyl-2,3,4,5-tetrahydropyridazin-3-one;

2-(4-pyrazinecarbonylaminobenzyl)-6-(3-methoxy-4-trifluoromethoxyphenyl)-5-ethyl-2,3,4,5-tetrahydropyridazin-3-one;

2-(4-pyrazinecarbonylaminobenzyl)-6-(3-methoxy-4-difluoromethoxyphenyl)-5-ethyl-2,3,4,5-tetrahydropyridazin-3-one;

2-(4-pyrazinecarbonylaminobenzyl)-6-(3-methoxy-4-fluoromethoxyphenyl)-5-ethyl-2,3,4,5-tetrahydropyridazin-3-one;

2-(4-pyrazinecarbonylaminobenzyl)-6-(3-difluoromethoxy-4-methoxyphenyl)-5-ethyl-2,3,4,5-tetrahydropyridazin-3-one;

2-(4-pyrazinecarbonylaminobenzyl)-6-(3-trifluoromethoxy-4-methoxyphenyl)-5-ethyl-2,3,4,5-tetrahydropyridazin-3-one;

2-(4-pyrazinecarbonylaminobenzyl)-6-(3-fluoromethoxy-4-methoxyphenyl)-5-ethyl-2,3,4,5-tetrahydropyridazin-3-one;

2-(4-pyrazinecarbonylaminobenzyl)-6-(3-methoxy-4-ethoxyphenyl)-5-ethyl-2,3,4,5-tetrahydropyridazin-3-one;

2-(4-pyrazinecarbonylaminobenzyl)-6-(3-ethoxy-4-methoxyphenyl)-5-ethyl-2,3,4,5-tetrahydropyridazin-3-one;

2-(4-pyrazinecarbonylaminobenzyl)-6-(3-hydroxy-4-methoxyphenyl)-5-ethyl-2,3,4,5-tetrahydropyridazin-3-one;

2-(4-pyrazinecarbonylaminobenzyl)-6-(4-methylsulfonylphenyl)-5-ethyl-2,3,4,5-tetrahydropyridazin-3-one;

2-(4-pyrazinecarbonylaminobenzyl)-6-(4-methyleneoxyphenyl)-5-ethyl-2,3,4,5-tetrahydropyridazin-3-one;

2-(4-pyrazinecarbonylaminobenzyl)-6-(3-cyclopentyloxy-4-methoxyphenyl)-5-ethyl-2,3,4,5-tetrahydropyridazin-3-one;

2-(3-pyrazinecarbonylaminobenzyl)-6-(3-cyclopentyloxy-4-methoxyphenyl)-5-ethyl-2,3,4,5-tetrahydropyridazin-3-one;

2-(4-pyrazinecarbonylaminophenethyl)-6-(3,4-dimethoxyphenyl)-2,3,4,5-tetrahydropyridazin-3-one;

2-(4-pyrazinecarbonylaminophenethyl)-6-(3,4-dimethoxyphenyl)-5-ethyl-2,3,4,5-tetrahydropyridazin-3-one;

3-(4-pyrazinecarbonylaminobenzyl)-5-(3,4-dimethoxyphenyl)-3,6-dihydro-1,3,4-thiadiazin-2-one, m.p. 202°;

3-(3-pyrazinecarbonylaminobenzyl)-5-(3,4-dimethoxyphenyl)-3,6-dihydro-1,3,4-thiadiazin-2-one;

3-(2-pyrazinecarbonylaminobenzyl)-5-(3,4-dimethoxyphenyl)-3,6-dihydro-1,3,4-thiadiazin-2-one;

3-(4-pyrazinecarbonylaminobenzyl)-5-(3,4-dimethoxyphenyl)-6-ethyl-3,6-dihydro-1,3,4-thiadiazin-2-one;

3-(3-pyrazinecarbonylaminobenzyl)-5-(3,4-dimethoxyphenyl)-6-ethyl-3,6-dihydro-1,3,4-thiadiazin-2-one;

3-(2-pyrazinecarbonylaminobenzyl)-5-(3,4-dimethoxyphenyl)-6-ethyl-3,6-dihydro-1,3,4-thiadiazin-2-one;

3-(4-pyrazinecarbonylaminobenzyl)-5-(3-methoxy-4-trifluoromethoxyphenyl)-6-ethyl-3,6-dihydro-1,3,4-thiadiazin-2-one;

3-(4-pyrazinecarbonylaminobenzyl)-5-(3-methoxy-4-difluoromethoxyphenyl)-6-ethyl-3,6-dihydro-1,3,4-thiadiazin-2-one;

3-(4-pyrazinecarbonylaminobenzyl)-5-(3-methoxy-4-fluoromethoxyphenyl)-6-ethyl-3,6-dihydro-1,3,4-thiadiazin-2-one;

3-(4-pyrazinecarbonylaminobenzyl)-5-(3-difluoromethoxy-4-methoxyphenyl)-6-ethyl-3,6-dihydro-1,3,4-thiadiazin-2-one,

3-(4-pyrazinecarbonylaminobenzyl)-5-(3-trifluoromethoxy-4-methoxyphenyl)-6-ethyl-3,6-dihydro-1,3,4-thiadiazin-2-one;

3-(4-pyrazinecarbonylaminobenzyl)-5-(3-fluoromethoxy-4-methoxyphenyl)-6-ethyl-3,6-dihydro-1,3,4-thiadiazin-2-one;

3-(4-pyrazinecarbonylaminobenzyl)-5-(3-methoxy-4-ethoxyphenyl)-6-ethyl-3,6-dihydro-1,3,4-thiadiazin-2-one;

3-(4-pyrazinecarbonylaminobenzyl)-5-(3-ethoxy-4-methoxyphenyl)-6-ethyl-3,6-dihydro-1,3,4-thiadiazin-2-one;

3-(4-pyrazinecarbonylaminobenzyl)-5-(3-hydroxy-4-methoxyphenyl)-6-ethyl-3,6-dihydro-1,3,4-thiadiazin-2-one;

3-(4-pyrazinecarbonylaminobenzyl)-5-(4-methylsulfonylphenyl)-6-ethyl-3,6-dihydro-1,3,4-thiadiazin-2-one;

3-(4-pyrazinecarbonylaminobenzyl)-5-(4-methyleneoxyphenyl)-6-ethyl-3,6-dihydro-1,3,4-thiadiazin-2-one;

3-(4-pyrazinecarbonylaminobenzyl)-5-(3-cyclopentyloxy-4-methoxyphenyl)-6-ethyl-3,6-dihydro-1,3,4-thiadiazin-2-one;

3-(3-pyrazinecarbonylaminobenzyl)-5-(3-cyclopentyloxy-4-methoxyphenyl)-6-ethyl-3,6-dihydro-1,3,4-thiadiazin-2-one;

3-(4-pyrazinecarbonylamino-phenethyl)-5-(3,4-dimethoxyphenyl)-3,6-dihydro-1,3,4-thiadiazin-2-one;

3-(4-pyrazinecarbonylaminophenethyl)-5-(3,4-dimethoxyphenyl)-6-ethyl-3,6-dihydro-1,3,4-thiadiazin-2-one;

3-(4-pyrazinecarbonylaminobenzyl)-5-(3,4-dimethoxyphenyl)-3,6-dihydro-1,3,4-oxadiazin-2-one;

3-(3-pyrazinecarbonylaminobenzyl)-5-(3,4-dimethoxyphenyl)-3,6-dihydro-1,3,4-oxadiazin-2-one;

3-(2-pyrazinecarbonylaminobenzyl)-5-(3,4-dimethoxyphenyl)-3,6-dihydro-1,3,4-oxadiazin-2-one;

3-(4-pyrazinecarbonylaminobenzyl)-5-(3,4-dimethoxyphenyl)-6-ethyl-3,6-dihydro-1,3,4-oxadiazin-2-one;

3-(3-pyrazinecarbonylaminobenzyl)-5-(3,4-dimethoxyphenyl)-6-ethyl-3,6-dihydro-1,3,4-oxadiazin-2-one;

3-(2-pyrazinecarbonylaminobenzyl)-5-(3,4-dimethoxyphenyl)-6-ethyl-3,6-dihydro-1,3,4-oxadiazin-2-one;

3-(4-pyrazinecarbonylaminobenzyl)-5-(3-methoxy-4-trifluoromethoxyphenyl)-6-ethyl-3,6-dihydro-1,3,4-oxadiazin-2-one;

3-(4-pyrazinecarbonylaminobenzyl)-5-(3-methoxy-4-difluoromethoxyphenyl)-6-ethyl-3,6-dihydro-1,3,4-oxadiazin-2-one,

3-(4-pyrazinecarbonylaminobenzyl)-5-(3-methoxy-4-fluoromethoxyphenyl)-6-ethyl-3,6-dihydro-1,3,4-oxadiazin-2-one;

3-(4-pyrazinecarbonylaminobenzyl)-5-(3-difluoromethoxy-4-methoxyphenyl)-6-ethyl-3,6-dihydro-1,3,4-oxadiazin-2-one;

3-(4-pyrazinecarbonylaminobenzyl)-5-(3-trifluoromethoxy-4-methoxyphenyl)-6-ethyl-3,6-dihydro-1,3,4-oxadiazin-2-one;

3-(4-pyrazinecarbonylaminobenzyl)-5-(3-fluoromethoxy-4-methoxyphenyl)-6-ethyl-3,6-dihydro-1,3,4-oxadiazin-2-one;

3-(4-pyrazinecarbonylaminobenzyl)-5-(3-methoxy-4-ethoxyphenyl)-6-ethyl-3,6-dihydro-1,3,4-oxadiazin-2-one;

3-(4-pyrazinecarbonylaminobenzyl)-5-(3-ethoxy-4-methoxyphenyl)-6-ethyl-3,6-dihydro-1,3,4-oxadiazin-2-one;

3-(4-pyrazinecarbonylaminobenzyl)-5-(3-hydroxy-4-methoxyphenyl)-6-ethyl-3,6-dihydro-1,3,4-oxadiazin-2-one;

3-(4-pyrazinecarbonylaminobenzyl)-5-(4-methylsulfonylphenyl)-6-ethyl-3,6-dihydro-1,3,4-oxadiazin-2-one;

3-(4-pyrazinecarbonylaminobenzyl)-5-(4-methyleneoxyphenyl)-6-ethyl-3,6-dihydro-1,3,4-oxadiazin-2-one;

3-(4-pyrazinecarbonylaminobenzyl)-5-(3-cyclopentyloxy-4-methoxyphenyl)-6-ethyl-3,6-dihydro-1,3,4-oxadiazin-2-one;

3-(3-pyrazinecarbonylaminobenzyl)-5-(3-cyclopentyloxy-4-methoxyphenyl)-6-ethyl-3,6-dihydro-1,3,4-oxadiazin-2-one;

3-(4-pyrazinecarbonylamino-phenethyl)-5-(3,4-dimethoxyphenyl)-3,6-dihydro-1,3,4-oxadiazin-2-one;

3-(4-pyrazinecarbonylamino-phenethyl)-5-(3,4-dimethoxyphenyl)-6-ethyl-3,6-dihydro-1,3,4-oxadiazin-2-one;

2-(4-(6-chloropyridazine-3-carbonylamino)benzyl)-6-(3,4-dimethoxyphenyl)-2,3,4,5-tetrahydropyridazin-3-one;

2-(3-(6-chloropyridazine-3-carbonylamino)benzyl)-6-(3,4-dimethoxyphenyl)-2,3,4,5-tetrahydropyridazin-3-one;

2-(2-(6-chloropyridazine-3-carbonylamino)benzyl)-6-(3,4-dimethoxyphenyl)-2,3,4,5-tetrahydropyridazin-3-one;

2-(4-(6-chloropyridazine-3-carbonylamino)benzyl)-6-(3,4-dimethoxyphenyl)-5-ethyl-2,3,4,5-tetrahydropyridazin-3-one;

2-(3-(6-chloropyridazine-3-carbonylamino)benzyl)-6-(3,4-dimethoxyphenyl)-5-ethyl-2,3,4,5-tetrahydropyridazin-3-one;

2-(2-(6-chloropyridazine-3-carbonylamino)benzyl)-6-(3,4-dimethoxyphenyl)-5-ethyl-2,3,4,5-tetrahydropyridazin-3-one;

2-(4-(6-chloropyridazine-3-carbonylamino)benzyl)-6-(3-methoxy-4-trifluoromethoxyphenyl)-5-ethyl-2,3,4,5-tetrahydropyridazin-3-one;

2-(4-(6-chloropyridazine-3-carbonylamino)benzyl)-6-(3-methoxy-4-difluoromethoxyphenyl)-5-ethyl-2,3,4,5-tetrahydropyridazin-3-one;

2-(4-(6-chloropyridazine-3-carbonylamino)benzyl)-6-(3-methoxy-4-fluoromethoxyphenyl)-5-ethyl-2,3,4,5-tetrahydropyridazin-3-one;

2-(4-(6-chloropyridazine-3-carbonylamino)benzyl)-6-(3-difluoromethoxy-4-methoxyphenyl)-5-ethyl-2,3,4,5-tetrahydropyridazin-3-one;

2-(4-(6-chloropyridazine-3-carbonylamino)benzyl)-6-(3-trifluoromethoxy-4-methoxyphenyl)-5-ethyl-2,3,4,5-tetrahydropyridazin-3-one;

2-(4-(6-chloropyridazine-3-carbonylamino)benzyl)-6-(3-fluoromethoxy-4-methoxyphenyl)-5-ethyl-2,3,4,5-tetrahydropyridazin-3-one;

2-(4-(6-chloropyridazine-3-carbonylamino)benzyl)-6-(3-methoxy-4-ethoxyphenyl)-5-ethyl-2,3,4,5-tetrahydropyridazin-3-one;

2-(4-(6-chloropyridazine-3-carbonylamino)benzyl)-6-(3-ethoxy-4-methoxyphenyl)-5-ethyl-2,3,4,5-tetrahydropyridazin-3-one;

2-(4-(6-chloropyridazine-3-carbonylamino)benzyl)-6-(3-hydroxy-4-methoxyphenyl)-5-ethyl-2,3,4,5-tetrahydropyridazin-3-one;

2-(4-(6-chloropyridazine-3-carbonylamino)benzyl)-6-(4-methylsulfonylphenyl)-5-ethyl-2,3,4,5-tetrahydropyridazin-3-one;

2-(4-(6-chloropyridazine-3-carbonylamino)benzyl)-6-(4-methyleneoxyphenyl)-5-ethyl-2,3,4,5-tetrahydropyridazin-3-one;

2-(4-(6-chloropyridazine-3-carbonylamino)benzyl)-6-(3-cyclopentyloxy-4-methoxyphenyl)-5-ethyl-2,3,4,5-tetrahydropyridazin-3-one;

2-(3-(6-chloropyridazine-3-carbonylamino)benzyl)-6-(3-cyclopentyloxy-4-methoxyphenyl)-5-ethyl-2,3,4,5-tetrahydropyridazin-3-one;

2-(4-(6-chloropyridazine-3-carbonylamino)phenethyl)-6-(3,4-dimethoxyphenyl)-2,3,4,5-tetrahydropyridazin-3-one;

2-(4-(6-chloropyridazine-3-carbonylamino)phenethyl)-6-(3,4-dimethoxyphenyl)-5-ethyl-2,3,4,5-tetrahydropyridazin-3-one;

3-(4-(6-chloropyridazine-3-carbonylamino)benzyl-5-(3,4-dimethoxyphenyl)-3,6-dihydro-1,3,4-thiadiazin-2-one;

3-(3-(6-chloropyridazine-3-carbonylamino)benzyl)-5-(3,4-dimethoxyphenyl)-3,6-dihydro-1,3,4-thiadiazin-2-one;

3-(2-(6-chloropyridazine-3-carbonylamino)benzyl)-5-(3,4-dimethoxyphenyl)-3,6-dihydro-1,3,4-thiadiazin-2-one;

3-(4-(6-chloropyridazine-3-carbonylamino)benzyl)-5-(3,4-dimethoxyphenyl)-6-ethyl-3,6-dihydro-1,3,4-thiadiazin-2-one;

3-(3-(6-chloropyridazine-3-carbonylamino)benzyl)-5-(3,4-dimethoxyphenyl)-6-ethyl-3,6-dihydro-1,3,4-thiadiazin-2-one;

3-(2-(6-chloropyridazine-3-carbonylamino)benzyl)-5-(3,4-dimethoxyphenyl)-6-ethyl-3,6-dihydro-1,3,4-thiadiazin-2-one;

3-(4-(6-chloropyridazine-3-carbonylamino)benzyl)-5-(3-methoxy-4-trifluoromethoxyphenyl)-6-ethyl-3,6-dihydro-1,3,4-thiadiazin-2-one;

3-(4-(6-chloropyridazine-3-carbonylamino)benzyl)-5-(3-methoxy-4-difluoromethoxyphenyl)-6-ethyl-3,6-dihydro-1,3,4-thiadiazin-2-one;

3-(4-(6-chloropyridazine-3-carbonylamino)benzyl)-5-(3-methoxy-4-fluoromethoxyphenyl)-6-ethyl-3,6-dihydro-1,3,4-thiadiazin-2-one;

3-(4-(6-chloropyridazine-3-carbonylamino)benzyl)-5-(3-difluoromethoxy-4-methoxyphenyl)-6-ethyl-3,6-dihydro-1,3,4-thiadiazin-2-one;

3-(4-(6-chloropyridazine-3-carbonylamino)benzyl)-5-(3-trifluoromethoxy-4-methoxyphenyl)-6-ethyl-3,6-dihydro-1,3,4-thiadiazin-2-one;

3-(4-(6-chloropyridazine-3-carbonylamino)benzyl)-5-(3-fluoromethoxy-4-methoxyphenyl)-6-ethyl-3,6-dihydro-1,3,4-thiadiazin-2-one;

3-(4-(6-chloropyridazine-3-carbonylamino)benzyl)-5-(3-methoxy-4-ethoxyphenyl)-6-ethyl-3,6-dihydro-1,3,4-thiadiazin-2-one;

3-(4-(6-chloropyridazine-3-carbonylamino)benzyl)-5-(3-ethoxy-4-methoxyphenyl)-6-ethyl-3,6-dihydro-1,3,4-thiadiazin-2-one;

3-(4-(6-chloropyridazine-3-carbonylamino)benzyl)-5-(3-hydroxy-4-methoxyphenyl)-6-ethyl-3,6-dihydro-1,3,4-thiadiazin-2-one;

3-(4-(6-chloropyridazine-3-carbonylamino)benzyl)-5-(4-methylsulfonylphenyl)-6-ethyl-3,6-dihydro-1,3,4-thiadiazin-2-one;

3-(4-(6-chloropyridazine-3-carbonylamino)benzyl)-5-(4-methyleneoxyphenyl)-6-ethyl-3,6-dihydro-1,3,4-thiadiazin-2-one;

3-(4-(6-chloropyridazine-3-carbonylamino)benzyl)-5-(3-cyclopentyloxy-4-methoxyphenyl)-6-ethyl-3,6-dihydro-1,3,4-thiadiazin-2-one;

3-(3-(6-chloropyridazine-3-carbonylamino)benzyl)-5-(3-cyclopentyloxy-4-methoxyphenyl)-6-ethyl-3,6-dihydro-1,3,4-thiadiazin-2-one;

3-(4-(6-chloropyridazine-3-carbonylamino)phenethyl)-5-(3,4-dimethoxyphenyl)-3,6-dihydro-1,3,4-thiadiazin-2-one;

3-(4-(6-chloropyridazine-3-carbonylamino)phenethyl)-5-(3,4-dimethoxyphenyl)-6-ethyl-3,6-dihydro-1,3,4-thiadiazin-2-one;

3-(4-(6-chloropyridazine-3-carbonylamino)benzyl)-5-(3,4-dimethoxyphenyl)-3,6-dihydro-1,3,4-oxadiazin-2-one;

3-(3-(6-chloropyridazine-3-carbonylamino)benzyl)-5-(3,4-dimethoxyphenyl)-3,6-dihydro-1,3,4-oxadiazin-2-one;

3-(2-(6-chloropyridazine-3-carbonylamino)benzyl)-5-(3,4-dimethoxyphenyl)-3,6-dihydro-1,3,4-oxadiazin-2-one;

3-(4-(6-chloropyridazine-3-carbonylamino)benzyl)-5-(3,4-dimethoxyphenyl)-6-ethyl-3,6-dihydro-1,3,4-oxadiazin-2-one;

3-(3-(6-chloropyridazine-3-carbonylamino)benzyl)-5-(3,4-dimethoxyphenyl)-6-ethyl-3,6-dihydro-1,3,4-oxadiazin-2-one;

3-(2-(6-chloropyridazine-3-carbonylamino)benzyl)-5-(3-methoxy-4-trifluoromethoxyphenyl)-6-ethyl-3,6-dihydro-1,3,4-oxadiazin-2-one;

3-(4-(6-chloropyridazine-3-carbonylamino)benzyl)-5-(3-methoxy-4-difluoromethoxyphenyl)-6-ethyl-3,6-dihydro-1,3,4-oxadiazin-2-one;

3-(4-(6-chloropyridazine-3-carbonylamino)benzyl)-5-(3-methoxy-4-fluoromethoxyphenyl)-6-ethyl-3,6-dihydro-1,3,4-oxadiazin-2-one;

3-(4-(6-chloropyridazine-3-carbonylamino)benzyl)-5-(3-difluoromethoxy-4-methoxyphenyl)-6-ethyl-3,6-dihydro-1,3,4-oxadiazin-2-one;

3-(4-(6-chloropyridazine-3-carbonylamino)benzyl)-5-(3-trifluoromethoxy-4-methoxyphenyl)-6-ethyl-3,6-dihydro-1,3,4-oxadiazin-2-one;

3-(4-(6-chloropyridazine-3-carbonylamino)benzyl)-5-(3-fluoromethoxy-4-methoxyphenyl)-6-ethyl-3,6-dihydro-1,3,4-oxadiazin-2-one;

3-(4-(6-chloropyridazine-3-carbonylamino)benzyl)-5-(3-methoxy-4-ethoxyphenyl)-6-ethyl-3,6-dihydro-1,3,4-oxadiazin-2-one;

3-(4-(6-chloropyridazine-3-carbonylamino)benzyl)-5-(3-ethoxy-4-methoxyphenyl)-6-ethyl-3,6-dihydro-1,3,4-oxadiazin-2-one;

3-(4-(6-chloropyridazine-3-carbonylamino)benzyl)-5-(3-hydroxy-4-methoxyphenyl)-6-ethyl-3,6-dihydro-1,3,4-oxadiazin-2-one;

3-(4-(6-chloropyridazine-3-carbonylamino)benzyl)-5-(4-methylsulfonylphenyl)-6-ethyl-3,5-dihydro-1,3,4-oxadiazin-2-one;

3-(4-(6-chloropyridazine-3-carbonylamino)benzyl)-5-(4-methyleneoxyphenyl)-6-ethyl-3,6-dihydro-1,3,4-oxadiazin-2-one;

3-(4-(6-chloropyridazine-3-carbonylamino)benzyl)-5-(3-cyclopentyloxy-4-methoxyphenyl)-6-ethyl-3,6-dihydro-1,3,4-oxadiazin-2-one;

3-(3-(6-chloropyridazine-3-carbonylamino)benzyl)-5-(3-cyclopentyloxy-4-methoxyphenyl)-6-ethyl-3,6-dihydro-1,3,4-oxadiazin-2-one;

3-(4-(6-chloropyridazine-3-carbonylamino)phenethyl)-5-(3,4-dimethoxyphenyl)-3,6-dihydro-1,3,4-oxadiazin-2-one;

3-(4-(6-chloropyridazine-3-carbonylamino)phenethyl)-5-(3,4-dimethoxyphenyl)-6-ethyl-3,6-dihydro-1,3,4-oxadiazin-2-one;

2-(4-(imidazole-4-carbonylamino)benzyl)-6-(3,4-dimethoxyphenyl)-2,3,4,5-tetrahydropyridazin-3-one;

2-(3-(imidazole-4-carbonylamino)benzyl)-6-(3,4-dimethoxyphenyl)-2,3,4,5-tetrahydropyridazin-3-one;

2-(2-(imidazole-4-carbonylamino)benzyl)-6-(3,4-dimethoxyphenyl)-2,3,4,5-tetrahydropyridazin-3-one;

2-(4-(imidazole-4-carbonylamino)benzyl)-6-(3,4-dimethoxyphenyl)-5-ethyl-2,3,4,5-tetrahydropyridazin-3-one;

2-(3-(imidazole-4-carbonylamino)benzyl)-6-(3,4-dimethoxyphenyl)-5-ethyl-2,3,4,5-tetrahydropyridazin-3-one;

2-(2-(imidazole-4-carbonylamino)benzyl)-6-(3,4-dimethoxyphenyl)-5-ethyl-2,3,4,5-tetrahydropyridazin-3-one;

2-(4-(imidazole-4-carbonylamino)benzyl)-6-(3-methoxy-4-trifluoromethoxy-5-ethyl-2,3,4,5-tetrahydropyridazin-3-one;

2-(4-(imidazole-4-carbonylamino)benzyl)-6-(3-methoxy-4-difluoromethoxyphenyl)-5-ethyl-2,3,4,5-tetrahydropyridazin-3-one;

2-(4-(imidazole-4-carbonylamino)benzyl)-6-(3-methoxy-4-fluoromethoxyphenyl)-5-ethyl-2,3,4,5-tetrahydropyridazin-3-one;

2-(4-(imidazole-4-carbonylamino)benzyl)-6-(3-difluoromethoxy-4-methoxyphenyl)-5-ethyl-2,3,4,5-tetrahydropyridazin-3-one;

2-(4-(imidazole-4-carbonylamino)benzyl)-6-(3-trifluoromethoxy-4-methoxyphenyl)-5-ethyl-2,3,4,5-tetrahydropyridazin-3-one;

2-(4-(imidazole-4-carbonylamino)benzyl)-6-(3-fluoromethoxy-4-methoxyphenyl)-5-ethyl-2,3,4,5-tetrahydropyridazin-3-one;

2-(4-(imidazole-4-carbonylamino)benzyl)-6-(3-methoxy-4-ethoxyphenyl)-5-ethyl-2,3,4,5-tetrahydropyridazin-3-one;

2-(4-(imidazole-4-carbonylamino)benzyl)-6-(3-ethoxy-4-methoxyphenyl)-5-ethyl-2,3,4,5-tetrahydropyridazin-3-one;

2-(4-(imidazole-4-carbonylamino)benzyl)-6-(3-hydroxy-4-methoxyphenyl)-5-ethyl-2,3,4,5-tetrahydropyridazin-3-one;

2-(4-(imidazole-4-carbonylamino)benzyl)-6-(4-methylsulfonylphenyl)-5-methyl-2,3,4,5-tetrahydropyridazin-3-one;

2-(4-(imidazole-4-carbonylamino)benzyl)-6-(4-methyleneoxyphenyl)-5-ethyl-2,3,4,5-tetrahydropyridazin-3-one;

2-(4-(imidazole-4-carbonylamino)benzyl)-6-(3-cyclopentyloxy-4-methoxyphenyl)-5-ethyl-2,3,4,5-tetrahydropyridazin-3-one;

2-(3-(imidazole-4-carbonylamino)benzyl)-6-(3-cyclopentyloxy-4-methoxyphenyl)-5-ethyl-2,3,4,5-tetrahydropyridazin-3-one;

2-(4-(imidazole-4-carbonylamino)phenethyl)-6-(3,4-dimethoxyphenyl)-2,3,4,5-tetrahydropyridazin-3-one;

2-(4-(imidazole-4-carbonylamino)phenethyl)-6-(3,4-dimethoxyphenyl)-5-ethyl-2,3,4,5-tetrahydropyridazin-3-one;

3-(4-(imidazole-4-carbonylamino)benzyl)-5-(3,4-dimethoxyphenyl)-3,6-dihydro-1,3,4-thiadiazin-2-one;

3-(3-(imidazole-4-carbonylamino)benzyl)-5-(3,4-dimethoxyphenyl)-3,6-dihydro-1,3,4-thiadiazin-2-one;

3-(2-(imidazole-4-carbonylamino)benzyl)-5-(3,4-dimethoxyphenyl)-3,6-dihydro-1,3,4-thiadiazin-2-one;

3-(4-(imidazole-4-carbonylamino)benzyl)-5-(3,4-dimethoxyphenyl)-6-ethyl-3,6-dihydro-1,3,4-thiadiazin-2-one;

3-(3-(imidazole-4-carbonylamino)benzyl)-5-(3,4-dimethoxyphenyl)-6-ethyl-3,6-dihydro-1,3,4-thiadiazin-2-one;

3-(2-(imidazole-4-carbonylamino)benzyl)-5-(3,4-dimethoxyphenyl)-6-ethyl-3,6-dihydro-1,3,4-thiadiazin-2-one;

3-(4-(imidazole-4-carbonylamino)benzyl)-5-(3-methoxy-4-trifluoromethoxyphenyl)-6-ethyl-3,6-dihydro-1,3,4-thiadiazin-2-one;

3-(4-(imidazole-4-carbonylamino)benzyl)-5-(3-methoxy-4-difluoromethoxyphenyl)-6-ethyl-3,6-dihydro-1,3,4-thiadiazin-2-one;

3-(4-(imidazole-4-carbonylamino)benzyl)-5-(3-methoxy-4-fluoromethoxyphenyl)-6-ethyl-3,6-dihydro-1,3,4-thiadiazin-2-one;

3-(4-(imidazole-4-carbonylamino)benzyl)-5-(3-difluoromethoxy-4-methoxyphenyl)-6-ethyl-3,6-dihydro-1,3,4-thiadiazin-2-one;

3-(4-(imidazole-4-carbonylamino)benzyl)-5-(3-trifluoromethoxy-4-methoxyphenyl)-6-ethyl-3,6-dihydro-1,3,4-thiadiazin-2-one;

3-(4-(imidazole-4-carbonylamino)benzyl)-5-(3-fluoromethoxy-4-methoxyphenyl)-6-ethyl-3,6-dihydro-1,3,4-thiadiazin-2-one;

3-(4-(imidazole-4-carbonylamino)benzyl)-5-(3-methoxy-4-ethoxyphenyl)-6-ethyl-3,6-dihydro-1,3,4-thiadiazin-2-one;

3-(4-(imidazole-4-carbonylamino)benzyl)-5-(3-ethoxy-4-methoxyphenyl)-6-ethyl-3,6-dihydro-1,3,4-thiadiazin-2-one;

3-(4-(imidazole-4-carbonylamino)benzyl)-5-(3-hydroxy-4-methoxyphenyl)-6-ethyl-3,6-dihydro-1,3,4-thiadiazin-2-one;

3-(4-(imidazole-4-carbonylamino)benzyl)-5-(4-methylsulfonylphenyl)-6-ethyl-3,6-dihydro-1,3,4-thiadiazin-2-one;

3-(4-(imidazole-4-carbonylamino)benzyl)-5-(4-methyleneoxyphenyl)-6-ethyl-3,6-dihydro-1,3,4-thiadiazin-2-one;

3-(4-(imidazole-4-carbonylamino)benzyl)-5-(3-cyclopentyloxy-4-methoxyphenyl)-6-ethyl-3,6-dihydro-1,3,4-thiadiazin-2-one;

3-(3-(imidazole-4-carbonylamino)benzyl)-5-(3-cyclopentyloxy-4-methoxyphenyl)-6-ethyl-3,6-dihydro-1,3,4-thiadiazin-2-one;

3-(4-(imidazole-4-carbonylamino)phenethyl)-5-(3,4-dimethoxyphenyl)-3,6-dihydro-1,3,4-thiadiazin-2-one;

3-(4-(imidazole-4-carbonylamino)phenethyl)-5-(3,4-dimethoxyphenyl)-6-ethyl-3,6-dihydro-1,3,4-thiadiazin-2-one;

3-(4-(imidazole-4-carbonylamino)benzyl)-5-(3,4-dimethoxyphenyl)-3,5-dihydro-1,3,4-oxadiazin-2-one;

3-(3-(imidazole-4-carbonylamino)benzyl)-5-(3,4-dimethoxyphenyl)-3,6-dihydro-1,3,4-oxadiazin-2-one;

3-(2-(imidazole-4-carbonylamino)benzyl)-5-(3,4-dimethoxyphenyl)-3,6-dihydro-1,3,4-oxadiazin-2-one;

3-(4-(imidazole-4-carbonylamino)benzyl)-5-(3,4-dimethoxyphenyl)-6-ethyl-3,6-dihydro-1,3,4-oxadiazin-2-one;

3-(3-(imidazole-4-carbonylamino)benzyl)-5-(3,4-dimethoxyphenyl)-6-ethyl-3,6-dihydro-1,3,4-oxadiazin-2-one;

3-(2-(imidazole-4-carbonylamino)benzyl)-5-(3,4-dimethoxyphenyl)-6-ethyl-3,6-dihydro-1,3,4-oxadiazin-2-one;

3-(4-(imidazole-4-carbonylamino)benzyl)-5-(3-methoxy-4-trifluoromethoxyphenyl)-6-ethyl-3,6-dihydro-1,3,4-oxadiazin-2-one;

3-(4-(imidazole-4-carbonylamino)benzyl)-5-(3-methoxy-4-difluoromethoxyphenyl)-6-ethyl-3,6-dihydro-1,3,4-oxadiazin-2-one;

3-(4-(imidazole-4-carbonylamino)benzyl)-5-(3-methoxy-4-fluoromethoxyphenyl)-6-ethyl-3,6-dihydro-1,3,4-oxadiazin-2-one;

3-(4-(imidazole-4-carbonylamino)benzyl)-5-(3-difluoromethoxy-4-methoxyphenyl)-6-ethyl-3,6-dihydro-1,3,4-oxadiazin-2-one;

3-(4-(imidazole-4-carbonylamino)benzyl)-5-(3-trifluoromethoxy-4-methoxyphenyl)-6-ethyl-3,6-dihydro-1,3,4-oxadiazin-2-one;

3-(4-(imidazole-4-carbonylamino)benzyl)-5-(3-fluoromethoxy-4-methoxyphenyl)-6-ethyl-3,6-dihydro-1,3,4-oxadiazin-2-one;

3-(4-(imidazole-4-carbonylamino)benzyl)-5-(3-methoxy-4-ethoxyphenyl)-6-ethyl-3,6-dihydro-1,3,4-oxadiazin-2-one;

3-(4-(imidazole-4-carbonylamino)benzyl)-5-(3-ethoxy-4-methoxyphenyl)-6-ethyl-3,6-dihydro-1,3,4-oxadiazin-2-one;

3-(4-(imidazole-4-carbonylamino)benzyl)-5-(3-hydroxy-4-methoxyphenyl)-6-ethyl-3,6-dihydro-1,3,4-oxadizin-2-one;

3-(4-(imidazole-4-carbonylamino)benzyl)-5-(4-methylsulfonylphenyl)-6-ethyl-3,6-dihydro-1,3,4-oxadiazin-2-one;

3-(4-(imidazole-4-carbonylamino)benzyl)-5-(4-methyleneoxyphenyl)-6-ethyl-3,6-dihydro-1,3,4-oxadiazin-2-one;

3-(4-(imidazole-4-carbonylamino)benzyl)-5-(3-cyclopentyloxy-4-methoxyphenyl)-6-ethyl-3,6-dihydro-1,3,4-oxadiazin-2-one;

3-(3-(imidazole-4-carbonylamino)benzyl)-5-(3-cyclopentyloxy-4-methoxyphenyl)-6-ethyl-3,6-dihydro-1,3,4-oxadiazin-2-one;

3-(4-(imidazole-4-carbonylamino)phenethyl)-5-(3,4-dimethoxyphenyl)-3,6-dihydro-1,3,4-oxadiazin-2-one;

3-(4-(imidazole-4-carbonylamino)phenethyl)-5-(3,1-dimethoxyphenyl)-6-ethyl-3,6-dihydro-1,3,4-oxadiazin-2-one;

2-(4-(2,4-dimethylthiazole-5-carbonylamino)benzyl)-6-(3,4-dimethoxyphenyl)-2,3,4,5-tetrahydropyridazin-3-one;

2-(4-(2,4-dimethylthiazole-5-carbonylamino)benzyl)-6-(3,4-dimethoxyphenyl)-2,3,4,5-tetrahydropyridazin-3-one;

2-(3-(2,4-dimethylthiazole-5-carbonylamino)benzyl)-6-(3,4-dimethoxyphenyl)-2,3,4,5-tetrahydropyridazin-3-one;

2-(4-(2,4-dimethylthiazole-5-carbonylamino)benzyl)-6-(3,4-dimethoxyphenyl)-5-ethyl-2,3,4,5-tetrahydropyridazin-3-one;

2-(3-(2,4-dimethylthiazole-5-carbonylamino)benzyl)-6-(3,4-dimethoxyphenyl)-5-ethyl-2,3,4,5-tetrahydropyridazin-3-one;

2-(3-(2,4-dimethylthiazole-5-carbonylamino)benzyl)-6-(3,4-dimethoxyphenyl)-5-ethyl-2,3,4,5-tetrahydropyridazin-3-one;

2-(2-(2,4-dimethylthiazole-5-carbonylamino)benzyl)-6-(3-methoxy-4-trifluoromethoxyphenyl)-5-ethyl-2,3,4,5-tetrahydropyridazin-3-one;

2-(4-(2,4-dimethylthiazole-5-carbonylamino)benzyl)-6-(3-methoxy-4-difluoromethoxyphenyl)-5-ethyl-2,3,4,5-tetrahydropyridazin-3-one;

2-(4-(2,4-dimethylthiazole-5-carbonylamino)benzyl)-6-(3-methoxy-4-fluoromethoxyphenyl)-5-ethyl-2,3,4,5-tetrahydropyridazin-3-one;

2-(4-(2,4-dimethylthiazole-5-carbonylamino)benzyl)-6-(3-difluoromethoxy-4-methoxyphenyl)-5-ethyl-2,3,4,5-tetrahydropyridazin-3-one;

2-(4-(2,4-dimethylthiazole-5-carbonylamino)benzyl)-6-(3-trifluoromethoxy-4-methoxyphenyl)-5-ethyl-2,3,4,5-tetrahydropyridazin-3-one;

2-(4-(2,4-dimethylthiazole-5-carbonylamino)benzyl)-6-(3-fluoromethoxy-4-methoxyphenyl)-5-ethyl-2,3,4,5-tetrahydropyridazin-3-one;

2-(4-(2,4-dimethylthiazole-5-carbonylamino)benzyl)-6-(3-methoxy-4-ethoxyphenyl)-5-ethyl-2,3,4,5-tetrahydropyridazin-3-one;

2-(4-(2,4-dimethylthiazole-5-carbonylamino)benzyl)-6-(3-ethoxy-4-methoxyphenyl)-5-ethyl-2,3,4,5-tetrahydropyridazin-3-one;

2-(4-(2,4-dimethylthiazole-5-carbonylamino)benzyl)-6-(3-hydroxy-4-methoxyphenyl)-5-ethyl-2,3,4,5-tetrahydropyridazin-3-one;

2-(4-(2,4-dimethylthiazole-5-carbonylamino)benzyl)-6-(4-methylsulfonylphenyl)-5-ethyl-2,3,4,5-tetrahydropyridazin-3-one;

2-(4-(2,4-dimethylthiazole-5-carbonylamino)benzyl)-6-(4-methyleneoxyphenyl)-5-ethyl-2,3,4,5-tetrahydropyridazin-3-one;

2-(4-(2,4-dimethylthiazole-5-carbonylamino)benzyl)-6-(3-cyclopentyloxy-4-methoxyphenyl)-5-ethyl-2,3,4,5-tetrahydropyridazin-3-one;

2-(3-(2,4-dimethylthiazole-5-carbonylamino)benzyl)-6-(3-cyclopentyloxy-4-methoxyphenyl)-5-ethyl-2,3,4,5-tetrahydropyridazin-3-one;

2-(4-(2,4-dimethylthiazole-5-carbonylamino)phenethyl)-6-(3,4-dimethoxyphenyl)-2,3,4,5-tetrahydropyridazin-3-one;

2-(4-(2,4-dimethylthiazole-5-carbonylamino)phenethyl)-6-(3,4-dimethoxyphenyl)-2,3,4,5-tetrahydropyridazin-3-one;

3-(4-(2,4-dimethylthiazole-5-carbonylamino)benzyl)-5-(3,4-dimethoxyphenyl)-3,6-dihydro-1,3,4-thiadiazin-2-one;

3-(3-(2,4-dimethylthiazole-5-carbonylamino)benzyl)-5-(3,4-dimethoxyphenyl)-3,6-dihydro-1,3,4-thiadiazin-2-one;

3-(2-(2,4-dimethylthiazole-5-carbonylamino)benzyl)-5-(3,4-dimethoxyphenyl)-3,6-dihydro-1,3,4-thiadiazin-2-one;

3-(4-(2,4-dimethylthiazole-5-carbonylamino)benzyl)-5-(3,4-dimethoxyphenyl)-6-ethyl-3,6-dihydro-1,3,4-thiadiazin-2-one;

3-(3-(2,4-dimethylthiazole-5-carbonylamino)benzyl)-5-(3,4-dimethoxyphenyl)-6-ethyl-3,6-dihydro-1,3,4-thiadiazin-2-one;

3-(2-(2,4-dimethylthiazole-5-carbonylamino)benzyl)-5-(3,4-dimethoxyphenyl)-6-ethyl-3,6-dihydro-1,3,4-thiadiazin-2-one;

3-(4-(2,4-dimethylthiazole-5-carbonylamino)benzyl)-5-(3-methoxy-4-trifluoromethoxyphenyl)-6-ethyl-3,6-dihydro-1,3,4-thiadiazin-2-one;

3-(4-(2,4-dimethylthiazole-5-carbonylamino)benzyl)-5-(3-methoxy-4-difluoromethoxyphenyl)-6-ethyl-3,6-dihydro-1,3,4-thiadiazin-2-one;

3-(4-(2,4-dimethylthiazole-5-carbonylamino)benzyl)-5-(3-methoxy-4-fluoromethoxyphenyl)-6-ethyl-3,6-dihydro-1,3,4-thiadiazin-2-one;

3-(4-(2,4-dimethylthiazole-5-carbonylamino)benzyl)-5-(3-difluoromethoxy-4-methoxyphenyl)-6-ethyl-3,6-dihydro-1,3,4-thiadiazin-2-one;

3-(4-(2,4-dimethylthiazole-5-carbonylamino)benzyl)-5-(3-trifluoromethoxy-4-methoxyphenyl)-6-ethyl-3,6-dihydro-1,3,4-thiadiazin-2-one;

3-(4-(2,4-dimethylthiazole-5-carbonylamino)benzyl)-5-(3-fluoromethoxy-4-methoxyphenyl)-6-ethyl-3,6-dihydro-1,3,4-thiadiazin-2-one;

3-(4-(2,4-dimethylthiazole-5-carbonylamino)benzyl)-5-(3-methoxy-4-ethoxyphenyl)-6-ethyl-3,6-dihydro-1,3,4-thiadiazin-2-one;

3-(4-(2,4-dimethylthiazole-5-carbonylamino)benzyl)-5-(3-ethoxy-4-methoxyphenyl)-6-ethyl-3,6-dihydro-1,3,4-thiadiazin-2-one;

3-(4-(2,4-dimethylthiazole-5-carbonylamino)benzyl)-5-(3-hydroxy-4-methoxyphenyl)-6-ethyl-3,6-dihydro-1,3,4-thiadiazin-2-one;

3-(4-(2,4-dimethylthiazole-5-carbonylamino)benzyl)-5-(4-methylsulfonylphenyl)-6-ethyl-3,6-dihydro-1,3,4-thiadiazin-2-one;

3-(4-(2,4-dimethylthiazole-5-carbonylamino)benzyl)-5-(4-methyleneoxyphenyl)-6-ethyl-3,6-dihydro-1,3,4-thiadiazin-2-one;

3-(3-(2,4-dimethylthiazole-5-carbonylamino)benzyl)-5-(3-cyclopentyloxy-4-methoxyphenyl)-6-ethyl-3,6-dihydro-1,3,4-thiadiazin-2-one;

3-(4-(2,4-dimethylthiazole-5-carbonylamino)benzyl)-5-(3-cyclopentyloxy-4-methoxyphenyl)-6-ethyl-3,6-dihydro-1,3,4-thiadiazin-2-one;

3-(4-(2,4-dimethylthiazole-5-carbonylamino)phenethyl)-5-(3,4-dimethoxyphenyl)-3,6-dihydro-1,3,4-thiadiazin-2-one;

3-(4-(2,4-dimethylthiazole-5-carbonylamino)phenethyl)-5-(3,4-dimethoxyphenyl)-6-ethyl-3,6-dihydro-1,3,4-thiadiazin-2-one;

3-(4-(2,4-dimethylthiazole-5-carbonylamino)benzyl)-5-(3,4-dimethoxyphenyl)-3,6-dihydro-1,3,4-oxadiazin-2-one;

3-(3-(imidazole-4-carbonylamino)benzyl)-5-(3,4-dimethoxyphenyl)-3,6-dihydro-1,3,4-oxadiazin-2-one;

3-(2-(2,4-dimethylthiazole-5-carbonylamino)benzyl)-5-(3,4-dimethoxyphenyl)-3,6-dihydro-1,3,4-oxadiazin-2-one;

3-(4-(2,4-dimethylthiazole-5-carbonylamino)benzyl)-5-(3,4-dimethoxyphenyl)-6-ethyl-3,4-dihydro-1,3,4-oxadiazin-2-one;

3-(3-(2,4-dimethylthiazole-5-carbonylamino)benzyl)-5-(3,4-dimethoxyphenyl)-6-ethyl-3,6-dihydro-1,3,4-oxadiazin-2-one;

3-(2-(2,4-dimethylthiazole-5-carbonylamino)benzyl)-5-(3,4-dimethoxyphenyl)-6-ethyl-3,6-dihydro-1,3,4-oxadiazin-2-one;

3-(4-(2,4-dimethylthiazole-5-carbonylamino)benzyl)-5-(3-methoxy-4-trifluoromethoxyphenyl)-6-ethyl-3,6-dihydro-1,3,4-oxadiazin-2-one;

3-(4-(2,4-dimethylthiazole-5-carbonylamino)benzyl)-5-(3-methoxy-4-difluoromethoxyphenyl)-6-ethyl-3,6-dihydro-1,3,4-oxadiazin-2-one;

3-(4-(2,4-dimethylthiazole-5-carbonylamino)benzyl)-5-(3-methoxy-4-fluoromethoxyphenyl)-6-ethyl-3,6-dihydro-1,3,4-oxadiazin-2-one;

3-(4-(2,4-dimethylthiazole-5-carbonylamino)benzyl)-5-(3-difluoromethoxy-4-methoxyphenyl)-6-ethyl-3,6-dihydro-1,3,4-oxadiazin-2-one;

3-(4-(2,4-dimethylthiazole-5-carbonylamino)benzyl)-5-(3-trifluoromethoxy-4-methoxyphenyl)-6-ethyl-3,6-dihydro-1,3,4-oxadiazin-2-one;

3-(4-(2,4-dimethylthiazole-5-carbonylamino)benzyl)-5-(3-fluoromethoxy-4-methoxyphenyl)-6-ethyl-3,6-dihydro-1,3,4-oxadiazin-2-one;

3-(4-(2,4-dimethylthiazole-5-carbonylamino)benzyl)-5-(3-methoxy-4-ethoxyphenyl)-6-ethyl-3,6-dihydro-1,3,4-oxadiazin-2-one;

3-(4-(2,4-dimethylthiazole-5-carbonylamino)benzyl)-5-(3-ethoxy-4-methoxyphenyl)-6-ethyl-3,6-dihydro-1,3,4-oxadiazin-2-one;

3-(4-(2,4-dimethylthiazole-5-carbonylamino)benzyl)-5-(3-hydroxy-4-methoxyphenyl)-6-ethyl-3,6-dihydro-1,3,4-oxadiazin-2-one;

3-(4-(2,4-dimethylthiazole-5-carbonylamino)benzyl-5-(4-methylsulfonylphenyl)-6-ethyl-3,6-dihydro-1,3,4-oxadiazin-2-one;

3-(4-(2,4-dimethylthiazole-5-carbonylamino)benzyl)-5-(4-methyleneoxyphenyl)-6-ethyl-3,6-dihydro-1,3,4-oxadiazin-2-one;

3-(4-(2,4-dimethylthiazole-5-carbonylamino)benzyl)-5-(3-cyclopentyloxy-4-methoxyphenyl)-6-ethyl-3,6-dihydro-1,3,4-oxadiazin-2-one;

3-(3-(2,4-dimethylthiazole-5-carbonylamino)benzyl)-5-(3-cyclopentyloxy-4-methoxyphenyl)-6-ethyl-3,6-dihydro-1,3,4-oxadiazin-2-one;

3-(4-(2,4-dimethylthiazole-5-carbonylamino)phenethyl)-5-(3,4-dimethoxyphenyl)-3,6-dihydro-1,3,4-oxadiazin-2-one;

3-(4-(2,4-dimethylthiazole-5-carbonylamino)phenethyl)-5-(3,4-dimethoxyphenyl)-6-ethyl-3,6-dihydro-1,3,4-oxadiazin-2-one;

2-(4-(isoxazole-5-carbonylamino)benzyl)-6-(3,4-dimethoxyphenyl)-2,3,4,5-tetrahydropyridazin-3-one, m.p. 217°;

2-(3-(isoxazole-5-carbonylamino)benzyl)-6-(3,4-dimethoxyphenyl)-2,3,4,5-tetrahydropyridazin-3-one;

2-(2-(isoxazole-5-carbonylamino)benzyl)-6-(3,4-dimethoxyphenyl)-2,3,4,5-tetrahydropyridazin-3-one;

2-(4-(isoxazole-5-carbonylamino)benzyl)-6-(3,4-dimethoxyphenyl)-5-ethyl-2,3,4,5-tetrahydropyridazin-3-one;

2-(3-(isoxazole-5-carbonylamino)benzyl)-6-(3,4-dimethoxyphenyl)-5-ethyl-2,3,4,5-tetrahydropyridazin-3-one;

2-(2-(isoxazole-5-carbonylamino)benzyl)-6-(3,4-dimethoxyphenyl)-5-ethyl-2,3,4,5-tetrahydropyridazin-3-one;

2-(4-(isoxazole-5-carbonylamino)benzyl)-6-(3-methoxy-4-trifluoromethoxyphenyl)-5-ethyl-2,3,4,5-tetrahydropyridazin-3-one;

2-(4-(isoxazole-5-carbonylamino)benzyl)-6-(3-methoxy-4-difluoromethoxyphenyl)-5-ethyl-2,3,4,5-tetrahydropyridazin-3-one;

2-(4-(isoxazole-5-carbonylamino)benzyl)-6-(3-methoxy-4-fluoromethoxyphenyl)-5-ethyl-2,3,4,5-tetrahydropyridazin-3-one;

2-(isoxazole-5-carbonylamino)benzyl)-6-(3-difluoromethoxy-4-methoxyphenyl)-5-ethyl-2,3,4,5-tetrahydropyridazin-3-one;

2-(4-(isoxazole-5-carbonylamino)benzyl)-6-(3-trifluoromethoxy-4-methoxyphenyl)-5-ethyl-2,3,4,5-tetrahydropyridazin-3-one;

2-(4-(isoxazole-5-carbonylamino)benzyl)-6-(3-fluoromethoxy-4-methoxyphenyl)-5-ethyl-2,3,4,5-tetrahydropyridazin-3-one;

2-(4-(isoxazole-5-carbonylamino)benzyl)-6-(3-methoxy-4-ethoxyphenyl)-5-ethyl-2,3,4,5-tetrahydropyridazin-3-one;

2-(4-(isoxazole-5-carbonylamino)benzyl)-6-(3-ethoxy-4-methoxyphenyl)-5-ethyl-2,3,4,5-tetrahydropyridazin-3-one;

2-(4-(isoxazole-5-carbonylamino)benzyl)-6-(3-hydroxy-4-methoxyphenyl)-5-ethyl-2,3,4,5-tetrahydropyridazin-3-one;

2-(4-(isoxazole-5-carbonylamino)benzyl)-6-(4-methylsulfonylphenyl)-5-ethyl-2,3,4,5-tetrahydropyridazin-3-one;

2-(4-(isoxazole-5-carbonylamino)benzyl)-6-(4-methyleneoxyphenyl)-5-ethyl-2,3,4,5-tetrahydropyridazin-3-one;

2-(4-(isoxazole-5-carbonylamino)benzyl)-6-(3-cyclopentyloxy-4-methoxyphenyl)-5-ethyl-2,3,4,5-tetrahydropyridazin-3-one;

2-(3-(isoxazole-5-carbonylamino)benzyl)-6-(3-cyclopentyloxy-4-methoxyphenyl)-5-ethyl-2,3,4,5-tetrahydropyridazin-3-one;

2-(4-(isoxazole-5-carbonylamino)phenethyl)-6-(3,4-dimethoxyphenyl)-2,3,4,5-tetrahydropyridazin-3-one;

2-(4-(isoxazole-5-carbonylamino)phenethyl)-6-(3,4-dimethoxyphenyl)-5-ethyl-2,3,4,5-tetrahydropyridazin-3-one;

3-(4-(isoxazole-5-carbonylamino)benzyl)-5-(3,4-dimethoxyphenyl)-3,6-dihydro-1,3,4-thiadiazin-2-one, m.p. 196°;

3-(3-(isoxazole-5-carbonylamino)benzyl)-5-(3,4-dimethoxyphenyl)-3,6-dihydro-1,3,4-thiadiazin-2-one;

3-(2-(isoxazole-5-carbonylamino)benzyl)-5-(3,4-dimethoxyphenyl)-3,6-dihydro-1,3,4-thiadiazin-2-one;

3-(4-(isoxazole-5-carbonylamino)benzyl)-5-(3,4-dimethoxyphenyl)-6-ethyl-3,6-dihydro-1,3,4-thiadiazin-2-one;

3-(3-(isoxazole-5-carbonylamino)benzyl)-5-(3,4-dimethoxyphenyl)-6-ethyl-3,6-dihydro-1,3,4-thiadiazin-2-one;

3-(2-(isoxazole-5-carbonylamino)benzyl)-5-(3,4-dimethoxyphenyl)-6-ethyl-3,6-dihydro-1,3,4-thiadiazin-2-one;

3-(4-(isoxazole-5-carbonylamino)benzyl)-5-(3-methoxy-4-trifluoromethoxyphenyl)-6-ethyl-3,6-dihydro-1,3,4-thiadiazin-2-one;

3-(4-(isoxazole-5-carbonylamino)benzyl)-5-(3-methoxy-4-difluoromethoxyphenyl)-6-ethyl-3,6-dihydro-1,3,4-thiadiazin-2-one;

3-(4-(isoxazole-5-carbonylamino)benzyl)-5-(3-methoxy-4-fluoromethoxyphenyl)-6-ethyl-3,6-dihydro-1,3,4-thiadiazin-2-one;

3-(4-(isoxazole-5-carbonylamino)benzyl)-5-(3-difluoromethoxy-4-methoxyphenyl)-6-ethyl-3,6-dihydro-1,3,4-thiadiazin-2-one;

3-(4-(isoxazole-5-carbonylamino)benzyl)-5-(3-trifluoromethoxy-4-methoxyphenyl)-6-ethyl-3,6-dihydro-1,3,4-thiadiazin-2-one;

3-(4-(isoxazole-5-carbonylamino)benzyl)-5-(3-fluoromethoxy-4-methoxyphenyl)-6-ethyl-3,6-dihydro-1,3,4-thiadiazin-2-one;

3-(4-(isoxazole-5-carbonylamino)benzyl)-5-(3-methoxy-4-ethoxyphenyl)-6-ethyl-3,6-dihydro-1,3,4-thiadiazin-2-one;

3-(4-(isoxazole-5-carbonylamino)benzyl)-5-(3-ethoxy-4-methoxyphenyl)-6-ethyl-3,6-dihydro-1,3,4-thiadiazin-2-one;

3-(4-(isoxazole-5-carbonylamino)benzyl)-5-(3-hydroxy-4-methoxyphenyl)-6-ethyl-3,6-dihydro-1,3,4-thiadiazin-2-one;

3-(4-(isoxazole-5-carbonylamino)benzyl)-5-(4-methylsulfonylphenyl)-6-ethyl-3,6-dihydro-1,3,4-thiadiazin-2-one;

3-(4-(isoxazole-5-carbonylamino)benzyl)-5-(4-methyleneoxyphenyl)-6-ethyl-3,6-dihydro-1,3,4-thiadiazin-2-one;

3-(4-(isoxazole-5-carbonylamino)benzyl)-5-(3-cyclopentyloxy-4-methoxyphenyl)-6-ethyl-3,6-dihydro-1,3,4-thiadiazin-2-one;

3-(3-(isoxazole-5-carbonylamino)benzyl)-5-(3-cyclopentyloxy-4-methoxyphenyl)-6-ethyl-3,6-dihydro-1,3,4-thiadiazin-2-one;

3-(4-(isoxazole-5-carbonylamino)phenethyl)-5-(3,4-dimethoxyphenyl)-3,6-dihydro-1,3,4-thiadiazin-2-one;

3-(4-(isoxazole-5-carbonylamino)phenethyl)-5-(3,4-dimethoxyphenyl)-6-ethyl-3,6-dihydro-1,3,4-thiadiazin-2-one;

3-(4-(isoxazole-5-carbonylamino)benzyl)-5-(3,4-dimethoxyphenyl)-3,6-dihydro-1,3,4-oxadiazin-2-one;

3-(3-(isoxazole-5-carbonylamino)benzyl)-5-(3,4-dimethoxyphenyl)-3,6-dihydro-1,3,4-oxadiazin-2-one;

3-(2-(isoxazole-5-carbonylamino)benzyl)-5-(3,4-dimethoxyphenyl)-3,6-dihydro-1,3,4-oxadiazin-2-one;

3-(4-(isoxazole-5-carbonylamino)benzyl)-5-(3,4-dimethoxyphenyl)-6-ethyl-3,6-dihydro-1,3,4-oxadiazin-2-one;

3-(3-(isoxazole-5-carbonylamino)benzyl)-5-(3,4-dimethoxyphenyl)-6-ethyl-3,6-dihydro-1,3,4-oxadiazin-2-one;

3-(2-(isoxazole-5-carbonylamino)benzyl)-5-(3,4-dimethoxyphenyl)-6-ethyl-3,6-dihydro-1,3,4-oxadiazin-2-one;

3-(4-(isoxazole-5-carbonylamino)benzyl)-5-(3-methoxy-4-trifluoromethoxyphenyl)-6-ethyl-3,6-dihydro-1,3,4-oxadiazin-2-one;

3-(4-(isoxazole-5-carbonylamino)benzyl)-5-(3-methoxy-4-difluoromethoxyphenyl)-6-ethyl-3,6-dihydro-1,3,4-oxadiazin-2-one;

3-(4-(isoxazole-5-carbonylamino)benzyl)-5-(3-methoxy-4-fluoromethoxyphenyl)-6-ethyl-3,6-dihydro-1,3,4-oxadiazin-2-one;

3-(4-(isoxazole-5-carbonylamino)benzyl)-5-(3-difluoromethoxy-4-methoxyphenyl)-6-ethyl-3,6-dihydro-1,3,4-oxadiazin-2-one;

3-(4-(isoxazole-5-carbonylamino)benzyl)-5-(3-trifluoromethoxy-4-methoxyphenyl)-6-ethyl-3,6-dihydro-1,3,4-oxadiazin-2-one;

3-(4-(isoxazole-5-carbonylamino)benzyl)-5-(3-fluoromethoxy-4-methoxyphenyl)-6-ethyl-3,6-dihydro-1,3,4-oxadiazin-2-one;

3-(4-(isoxazole-5-carbonylamino)benzyl)-5-(3-methoxy-4-ethoxyphenyl)-6-ethyl-3,6-dihydro-1,3,4-oxadiazin-2-one;

3-(4-(isoxazole-5-carbonylamino)benzyl)-5-(3-ethoxy-4-methoxyphenyl)-6-ethyl-3,6-dihydro-1,3,4-oxadiazin-2-one;

3-(4-(isoxazole-5-carbonylamino)benzyl)-5-(3-hydroxy-4-methoxyphenyl)-6-ethyl-3,6-dihydro-1,3,4-oxadiazin-2-one;

3-(4-(isoxazole-5-carbonylamino)benzyl)-5-(4-methylsulfonylphenyl)-6-ethyl-3,6-dihydro-1,3,4-oxadiazin-2-one;

3-(4-(isoxazole-5-carbonylamino)benzyl)-5-(4-methyleneoxyphenyl)-6-ethyl-3,6-dihydro-1,3,4-oxadiazin-2-one;

3-(4-(isoxazole-5-carbonylamino)benzyl)-5-(3-cyclopentyloxy-4-methoxyphenyl)-6-ethyl-3,6-dihydro-1,3,4-oxadiazin-2-one;

3-(4-(isoxazole-5-carbonylamino)benzyl)-5-(3-cyclopentyloxy-4-methoxyphenyl)-6-ethyl-3,6-dihydro-1,3,4-oxadiazin-2-one;

3-(4-(isoxazole-5-carbonylamino)phenethyl)-5-(3,4-dimethoxyphenyl)-3,6-dihydro-1,3,4-oxadiazin-2-one;

3-(4-(isoxazole-5-carbonylamino)phenethyl)-5-(3,4-dimethoxyphenyl)-6-ethyl-3,6-dihydro-1,3,4-oxadiazin-2-one;

2-(4-(oxazole-5-carbonylamino)benzyl)-6-(3,4-dimethoxyphenyl)-2,3,4,5-tetrahydropyridazin-3-one;

2-(3-(oxazole-5-carbonylamino)benzyl)-6-(3,4-dimethoxyphenyl)-2,3,4,5-tetrahydropyridazin-3-one;

2-(2-(oxazole-5-carbonylamino)benzyl)-6-(3,4-dimethoxyphenyl)-2,3,4,5-tetrahydropyridazin-3-one;

2-(4-(oxazole-5-carbonylamino)benzyl)-6-(3,4-dimethoxyphenyl)-5-ethyl-2,3,4,5-tetrahydropyridazin-3-one;

2-(3-(oxazole-5-carbonylamino)benzyl)-6-(3,4-dimethoxyphenyl)-5-ethyl-2,3,4,5-tetrahydropyridazin-3-one;

2-(2-(oxazole-5-carbonylamino)benzyl)-6-(3,4-dimethoxyphenyl)-5-ethyl-2,3,4,5-tetrahydropyridazin-3-one;

2-(4-(oxazole-5-carbonylamino)benzyl)-6-(3-methoxy-4-trifluoromethoxyphenyl)-5-ethyl-2,3,4,5-tetrahydropyridazin-3-one;

2-(4-(oxazole-5-carbonylamino)benzyl)-6-(3-methoxy-4-difluoromethoxyphenyl)-5-ethyl-2,3,4,5-tetrahydropyridazin-3-one;

2-(4-(oxazole-5-carbonylamino)benzyl)-6-(3-methoxy-4-fluoromethoxyphenyl)-5-ethyl-2,3,4,5-tetrahydropyridazin-3-one;

2-(4-(oxazole-5-carbonylamino)benzyl)-6-(3-difluoromethoxy-4-methoxyphenyl)-5-ethyl-2,3,4,5-tetrahydropyridazin-3-one;

2-(4-(oxazole-5-carbonylamino)benzyl)-6-(3-trifluoromethoxy-4-methoxyphenyl)-5-ethyl-2,3,4,5-tetrahydropyridazin-3-one;

2-(4-(oxazole-5-carbonylamino)benzyl)-6-(3-fluoromethoxy-4-methoxyphenyl)-5-ethyl-2,3,4,5-tetrahydropyridazin-3-one;

2-(4-(oxazole-5-carbonylamino)benzyl)-6-(3-methoxy-4-ethoxyphenyl)-5-ethyl-2,3,4,5-tetrahydropyridazin-3-one;

2-(4-(oxazole-5-carbonylamino)benzyl)-6-(3-ethoxy-4-methoxyphenyl)-5-ethyl-2,3,4,5-tetrahydropyridazin-3-one;

2-(4-(oxazole-5-carbonylamino)benzyl)-6-(3-hydroxy-4-methoxyphenyl)-5-ethyl-2,3,4,5-tetrahydropyridazin-3-one;

2-(4-(oxazole-5-carbonylamino)benzyl)-6-(4-methylsulfonylphenyl)-5-ethyl-2,3,4,5-tetrahydropyridazin-3-one;

2-(4-(oxazole-5-carbonylamino)benzyl)-6-(4-methyleneoxyphenyl)-5-ethyl-2,3,4,5-tetrahydropyridazin-3-one;

2-(4-(oxazole-5-carbonylamino)benzyl)-6-(3-cyclopentyloxy-4-methoxyphenyl)-5-ethyl-2,3,4,5-tetrahydropyridazin-3-one;

2-(3-(oxazole-5-carbonylamino)benzyl)-6-(3-cyclopentyloxy-4-methoxyphenyl)-5-ethyl-2,3,4,5-tetrahydropyridazin-3-one;

2-(4-(oxazole-5-carbonylamino)phenethyl)-6-(3,4-dimethoxyphenyl)-2,3,4,5-tetrahydropyridazin-3-one;

2-(4-(oxazole-5-carbonylamino)phenethyl)-6-(3,4-dimethoxyphenyl)-5-ethyl-2,3,4,5-tetrahydropyridazin-3-one;

3-(4-(oxazole-5-carbonylamino)benzyl)-5-(3,4-dimethoxyphenyl)-3,6-dihydro-1,3,4-thiadiazin-2-one;

3-(3-(oxazole-5-carbonylamino)benzyl)-5-(3,4-dimethoxyphenyl)-3,6-dihydro-1,3,4-thiadiazin-2-one;

3-(2-(oxazole-5-carbonylamino)benzyl)-5-(3,4-dimethoxyphenyl)-3,6-dihydro-1,3,4-thiadiazin-2-one;

3-(4-(oxazole-5-carbonylamino)benzyl)-5-(3,4-dimethoxyphenyl)-6-ethyl-3,6-dihydro-1,3,4-thiadiazin-2-one;

3-(3-(oxazole-5-carbonylamino)benzyl)-5-(3,4-dimethoxyphenyl)-6-ethyl-3,6-dihydro-1,3,4-thiadiazin-2-one;

3-(2-(oxazole-5-carbonylamino)benzyl)-5-(3,4-dimethoxyphenyl)-6-ethyl-3,6-dihydro-1,3,4-thiadiazin-2-one;

3-(4-(oxazole-5-carbonylamino)benzyl)-5-(3-methoxy-4-trifluoromethoxyphenyl)-6-ethyl-3,6-dihydro-1,3,4-thiadiazin-2-one;

3-(4-(oxazole-5-carbonylamino)benzyl)-5-(3-methoxy-4-difluoromethoxyphenyl)-6-ethyl-3,6-dihydro-1,3,4-thiadiazin-2-one;

3-(4-(oxazole-5-carbonylamino)benzyl)-5-(3-methoxy-4-fluoromethoxyphenyl)-6-ethyl-3,6-dihydro-1,3,4-thiadiazin-2-one;

3-(4-(oxazole-5-carbonylamino)benzyl)-5-(3-difluoromethoxy-4-methoxyphenyl)-6-ethyl-3,6-dihydro-1,3,4-thiadiazin-2-one;

3-(4-(oxazole-5-carbonylamino)benzyl)-5-(3-trifluoromethoxy-4-methoxyphenyl)-6-ethyl-3,6-dihydro-1,3,4-thiadiazin-2-one;

3-(4-(oxazole-5-carbonylamino)benzyl)-5-(3-fluoromethoxy-4-methoxyphenyl)-6-ethyl-3,6-dihydro-1,3,4-thiadiazin-2-one;

3-(4-(oxazole-5-carbonylamino)benzyl)-5-(3-methoxy-4-ethoxyphenyl)-6-ethyl-3,6-dihydro-1,3,4-thiadiazin-2-one;

3-(4-(oxazole-5-carbonylamino)benzyl)-5-(3-ethoxy-4-methoxyphenyl)-6-ethyl-3,6-dihydro-1,3,4-thiadiazin-2-one;

3-(4-(oxazole-5-carbonylamino)benzyl)-5-(3-hydroxy-4-methoxyphenyl)-6-ethyl-3,6-dihydro-1,3,4-thiadiazin-2-one;

3-(4-(oxazole-5-carbonylamino)benzyl)-5-(4-methylsulfonylphenyl)-6-ethyl-3,6-dihydro-1,3,4-thiadiazin-2-one;

3-(4-(oxazole-5-carbonylamino)benzyl)-5-(4-methyleneoxyphenyl)-6-ethyl-3,6-dihydro-1,3,4-thiadiazin-2-one;

3-(4-(oxazole-5-carbonylamino)benzyl)-5-(3-cyclopentyloxy-4-methoxyphenyl)-6-ethyl-3,6-dihydro-1,3,4-thiadiazin-2-one;

3-(3-(oxazole-5-carbonylamino)benzyl)-5-(3-cyclopentyloxy-4-methoxyphenyl)-6-ethyl-3,6-dihydro-1,3,4-thiadiazin-2-one;

3-(4-(oxazole-5-carbonylamino)phenethyl)-5-(3,4-dimethoxyphenyl)-3,6-dihydro-1,3,4-thiadiazin-2-one;

3-(4-(oxazole-5-carbonylamino)phenethyl)-5-(3,4-dimethoxyphenyl)-6-ethyl-3,6-dihydro-1,3,4-thiadiazin-2-one;

3-(4-(oxazole-5-carbonylamino)benzyl)-5-(3,4-dimethoxyphenyl)-3,6-dihydro-1,3,4-oxadiazin-2-one;

3-(3-(oxazole-5-carbonylamino)-benzyl)-5-(3,4-dimethoxyphenyl)-3,5-dihydro-1,3,4-oxadiazin-2-one;

3-(2-(oxazole-5-carbonylamino)benzyl)-5-(3,4-dimethoxyphenyl)-3,6-dihydro-1,3,4-oxadiazin-2-one;

3-(4-(oxazole-5-carbonylamino)benzyl)-5-(3,4-dimethoxyphenyl)-6-ethyl-3,5-dihydro-1,3,4-oxadiazin-2-one;

3-(3-(oxazole-5-carbonylamino)benzyl)-5-(3,4-dimethoxyphenyl)-6-ethyl-3,6-dihydro-1,3,4-oxadiazin-2-one;

3-(2-(oxazole-5-carbonylamino)benzyl)-5-(3,4-dimethoxyphenyl)-6-ethyl-3,6-dihydro-1,3,4-oxadiazin-2-one;

3-(4-(oxazole-5-carbonylamino)benzyl)-5-(3-methoxy-4-trifluoromethoxyphenyl)-6-ethyl-3,6-dihydro-1,3,4-oxadiazin-2-one;

3-(4-(oxazole-5-carbonylamino)benzyl)-5-(3-methoxy-4-difluoromethoxyphenyl)-6-ethyl-3,6-dihydro-1,3,4-oxadiazin-2-one;

3-(4-(oxazole-5-carbonylamino)benzyl)-5-(3-methoxy-4-fluoromethoxyphenyl)-6-ethyl-3,6-dihydro-1,3,4-oxadiazin-2-one;

3-(4-(oxazole-5-carbonylamino)benzyl)-5-(3-difluoromethoxy-4-methoxyphenyl)-6-ethyl-3,6-dihydro-1,3,4-oxadiazin-2-one;

3-(4-(oxazole-5-carbonylamino)benzyl)-5-(3-trifluoromethoxy-4-methoxyphenyl)-6-ethyl-3,6-dihydro-1,3,4-oxadiazin-2-one;

3-(4-(oxazole-5-carbonylamino)benzyl)-5-(3-fluoromethoxy-4-methoxyphenyl)-6-ethyl-3,6-dihydro-1,3,4-oxadiazin-2-one;

3-(4-(oxazole-5-carbonylamino)benzyl)-5-(3-methoxy-4-ethoxyphenyl)-6-ethyl-3,6-dihydro-1,3,4-oxadiazin-2-one;

3-(4-(oxazole-5-carbonylamino)benzyl)-5-(3-ethoxy-4-methoxyphenyl)-6-ethyl-3,6-dihydro-1,3,4-oxadiazin-2-one;

3-(4-(oxazole-5-carbonylamino)benzyl)-5-(3-hydroxy-4-methoxyphenyl)-6-ethyl-3,6-dihydro-1,3,4-oxadiazin-2-one;

3-(4-(oxazole-5-carbonylamino)benzyl)-5-(4-methylsulfonylphenyl)-6-ethyl-3,6-dihydro-1,3,4-oxadiazin-2-one;

3-(4-(oxazole-5-carbonylamino)benzyl)-5-(4-methyleneoxyphenyl)-6-ethyl-3,6-dihydro-1,3,4-oxadiazin-2-one;

3-(4-(oxazole-5-carbonylamino)benzyl)-5-(3-cyclopentyloxy-4-methoxyphenyl)-6-ethyl-3,6-dihydro-1,3,4-oxadiazin-2-one;

3-(3-(oxazole-5-carbonylamino)benzyl)-5-(3-cyclopentyloxy-4-methoxyphenyl)-6-ethyl-3,6-dihydro-1,3,4-oxadiazin-2-one;

3-(4-(oxazole-5-carbonylamino)phenethyl)-5-(3,4-dimethoxyphenyl)-3,6-dihydro-1,3,4-oxadiazin-2-one;

3-(4-(oxazole-5-carbonylamino)phenethyl)-5-(3,4-dimethoxyphenyl)-6-ethyl-3,6-dihydro-1,3,4-oxadiazin-2-one;

2-(4-(pyrimidine-2-carbonylamino)benzyl)-6-(3,4-dimethoxyphenyl)-2,3,4,5-tetrahydropyridazin-3-one;

2-(3-(pyrimidine-2-carbonylamino)benzyl)-6-(3,4-dimethoxyphenyl)-2,3,4,5-tetrahydropyridazin-3-one;

2-(2-(pyrimidine-2-carbonylamino)benzyl)-6-(3,4-dimethoxyphenyl)-2,3,4,5-tetrahydropyridazin-3-one;

2-(4-(pyrimidine-2-carbonylamino)benzyl)-6-(3,4-dimethoxyphenyl)-5-ethyl-2,3,4,5-tetrahydropyridazin-3-one;

2-(3-(pyrimidine-2-carbonylamino)benzyl)-6-(3,4-dimethoxyphenyl)-5-ethyl-2,3,4,5-tetrahydropyridazin-3-one;

2-(2-(pyrimidine-2-carbonylamino)benzyl)-6-(3,4,-dimethoxyphenyl)-5-ethyl-2,3,4,5-tetrahydropyridazin-3-one;

2-(4-(pyrimidine-2-carbonylamino)benzyl)-6-(3-methoxy-4-trifluoromethoxyphenyl)-5-ethyl-2,3,4,5-tetrahydropyridazin-3-one;

2-(4-(pyrimidine-2-carbonylamino)benzyl)-6-(3-methoxy-4-difluoromethoxyphenyl)-5-ethyl-2,3,4,5-tetrahydropyridazin-3-one;

2-(4-(pyrimidine-2-carbonylamino)benzyl)-6-(3-methoxy-4-fluoromethoxyphenyl)-5-ethyl-2,3,4,5-tetrahydropyridazin-3-one;

2-(4-(pyrimidine-2-carbonylamino)benzyl)-6-(3-difluoromethoxy-4-methoxyphenyl)-5-ethyl-2,3,4,5-tetrahydropyridazin-3-one;

2-(4-(pyrimidine-2-carbonylamino)benzyl)-6-(3-trifluoromethoxy-4-methoxyphenyl)-5-ethyl-2,3,4,5-tetrahydropyridazin-3-one;

2-(4-(pyrimidine-2-carbonylamino)benzyl)-6-(3-fluoromethoxy-4-methoxyphenyl)-5-ethyl-2,3,4,5-tetrahydropyridazin-3-one;

2-(4-(pyrimidine-2-carbonylamino)benzyl)-6-(3-methoxy-4-ethoxyphenyl)-5-ethyl-2,3,4,5-tetrahydropyridazin-3-one;

2-(4-(pyrimidine-2-carbonylamino)benzyl)-6-(3-ethoxy-4-methoxyphenyl)-5-ethyl-2,3,4,5-tetrahydropyridazin-3-one;

2-(4-(pyrimidine-2-carbonylamino)benzyl)-6-(3-hydroxy-4-methoxyphenyl)-5-ethyl-2,3,4,5-tetrahydropyridazin-3-one;

2-(4-(pyrimidine-2-carbonylamino)benzyl)-6-(4-methylsulfonylphenyl)-5-ethyl-2,3,4,5-tetrahydropyridazin-3-one;

2-(4-(pyrimidine-2-carbonylamino)benzyl)-6-(4-methyleneoxyphenyl)-5-ethyl-2,3,4,5-tetrahydropyridazin-3-one;

2-(4-(pyrimidine-2-carbonylamino)benzyl)-6-(3-cyclopentyloxy-4-methoxyphenyl)-5-ethyl-2,3,4,5-tetrahydropyridazin-3-one;

2-(3-(pyrimidine-2-carbonylamino)benzyl)-6-(3-cyclopentyloxy-4-methoxyphenyl)-5-ethyl-2,3,4,5-tetrahydropyridazin-3-one;

2-(4-(pyrimidine-2-carbonylamino)phenethyl)-6-(3,4-dimethoxyphenyl)-2,3,4,5-tetrahydropyridazin-3-one;

2-(4-(pyrimidine-2-carbonylamino)phenethyl)-6-(3,4-dimethoxyphenyl)-5-ethyl-2,3,4,5-tetrahydropyridazin-3-one;

3-(4-(pyrimidine-2-carbonylamino)benzyl)-5-(3,4-dimethoxyphenyl)-3,6-dihydro-1,3,4-thiadiazin-2-one;

3-(3-(pyrimidine-2-carbonylamino)benzyl)-5-(3,4-dimethoxyphenyl)-3,6-dihydro-1,3,4-thiadiazin-2-one;

3-(2-(pyrimidine-2-carbonylamino)benzyl)-5-(3,4-dimethoxyphenyl)-3,6-dihydro-1,3,4-thiadiazin-2-one;

3-(4-(pyrimidine-2-carbonylamino)benzyl)-5-(3,4-dimethoxyphenyl)-6-ethyl-3,6-dihydro-1,3,4-thiadiazin-2-one;

3-(3-(pyrimidine-2-carbonylamino)benzyl)-5-(3,4-dimethoxyphenyl)-6-ethyl-3,6-dihydro-1,3,4-thiadiazin-2-one;

3-(2-(pyrimidine-2-carbonylamino)benzyl)-5-(3,4-dimethoxyphenyl)-6-ethyl-3,6-dihydro-1,3,4-thiadiazin-2-one;

3-(4-(pyrimidine-2-carbonylamino)benzyl)-5-(3-methoxy-4-trifluoromethoxyphenyl)-6-ethyl-3,6-dihydro-1,3,4-thiadiazin-2-one;

3-(4-(pyrimidine-2-carbonylamino)benzyl)-5-(3-methoxy-4-difluoromethoxyphenyl)-6-ethyl-3,6-dihydro-1,3,4-thiadiazin-2-one;

3-(4-(pyrimidine-2-carbonylamino)benzyl)-5-(3-methoxy-4-fluoromethoxyphenyl)-6-ethyl-3,6-dihydro-1,3,4,-thiadiazin-2-one;

3-(4-(pyrimidine-2-carbonylamino)benzyl)-5-(3-difluoromethoxy-4-methoxyphenyl)-6-ethyl-3,6-dihydro-1,3,4-thiadiazin-2-one;

3-(4-(pyrimidine-2-carbonylamino)benzyl)-5-(3-trifluoromethoxy-4-methoxyphenyl)-6-ethyl-3,6-dihydro-1,3,4-thiadiazin-2-one;

3-(4-(pyrimidine-2-carbonylamino)benzyl)-5-(3-fluoromethoxy-4-methoxyphenyl)-6-ethyl-3,6-dihydro-1,3,4-thiadiazin-2-one;

3-(4-(pyrimidine-2-carbonylamino)benzyl)-5-(3-methoxy-4-ethoxyphenyl)-6-ethyl-3,6-dihydro-1,3,4-thiadiazin-2-one;

3-(4-(pyrimidine-2-carbonylamino)benzyl)-5-(3-ethoxy-4-methoxyphenyl)-6-ethyl-3,6-dihydro-1,3,4-thiadiazin-2-one;

3-(4-(pyrimidine-2-carbonylamino)benzyl)-5-(3-hydroxy-4-methoxyphenyl)-6-ethyl-3,6-dihydro-1,3,4-thiadiazin-2-one;

3-(4-(pyrimidine-2-carbonylamino)benzyl)-5-(4-methylsulfonylphenyl)-6-ethyl-3,6-dihydro-1,3,4-thiadiazin-2-one;

3-(4-(pyrimidine-2-carbonylamino)benzyl)-5-(4-methyleneoxyphenyl)-6-ethyl-3,6-dihydro-1,3,4-thiadiazin-2-one;

3-(4-(pyrimidine-2-carbonylamino)benzyl)-5-(3-cyclopentyloxy-4-methoxyphenyl)-6-ethyl-3,6-dihydro-1,3,4-thiadiazin-2-one;

3-(3-(pyrimidine-2-carbonylamino)benzyl)-5-(3-cyclopentyloxy-4-methoxyphenyl)-6-ethyl-3,6-dihydro-1,3,4-thiadiazin-2-one;

3-(4-(pyrimidine-2-carbonylamino)phenethyl)-5-(3,4-dimethoxyphenyl)-3,6-dihydro-1,3,4-thiadiazin-2-one;

3-(4-(pyrimidine-2-carbonylamino)phenethyl)-5-(3,4-dimethoxyphenyl)-6-ethyl-3,6-dihydro-1,3,4-thiadiazin-2-one;

3-(4-(pyrimidine-2-carbonylamino)benzyl)-5-(3,4-dimethoxyphenyl)-3,6-dihydro-1,3,4-oxadiazin-2-one;

3-(3-(pyrimidine-2-carbonylamino)benzyl)-5-(3,4-dimethoxyphenyl)-3,6-dihydro-1,3,4-oxadiazin-2-one;

3-(3-(pyrimidine-2-carbonylamino)benzyl)-5-(3,4-dimethoxyphenyl)-3,6-dihydro-1,3,4-oxadiazin-2-one;

3-(4-(pyrimidine-2-carbonylamino)benzyl)-5-(3,4-dimethoxyphenyl)-6-ethyl-3,6-dihydro-1,3,4-oxadiazin-2-one;

3-(3-(pyrimidine-2-carbonylamino)benzyl)-5-(3,4-dimethoxyphenyl)-6-ethyl-3,6-dihydro-1,3,4-oxadiazin-2-one;

3-(2-(pyrimidine-2-carbonylamino)benzyl)-5-(3,4-dimethoxyphenyl)-6-ethyl-3,6-dihydro-1,3,4-oxadiazin-2-one;

3-(4-(pyrimidine-2-carbonylamino)benzyl)-5-(3-methoxy-4-trifluoromethoxyphenyl)-6-ethyl-3,6-dihydro-1,3,4-oxadiazin-2-one;

3-(4-(pyrimidine-2-carbonylamino)benzyl)-5-(3-methoxy-4-difluoromethoxyphenyl)-6-ethyl-3,6-dihydro-1,3,4-oxadiazin-2-one;

3-(4-(pyrimidine-2-carbonylamino)benzyl)-5-(3-methoxy-4-fluoromethoxyphenyl)-6-ethyl-3,6-dihydro-1,3,4-oxadiazin-2-one;

3-(4-(pyrimidine-2-carbonylamino)benzyl)-5-(3-difluoromethoxy-4-methoxyphenyl)-6-ethyl-3,6-dihydro-1,3,4-oxadiazin-2-one;

3-(4-(pyrimidine-2-carbonylamino)benzyl)-5-(3-trifluoromethoxy-4-methoxyphenyl)-6-ethyl-3,6-dihydro-1,3,4-oxadiazin-2-one;

3-(4-(pyrimidine-2-carbonylamino)benzyl)-5-(3-fluoromethoxy-4-methoxyphenyl)-6-ethyl-3,6-dihydro-1,3,4-oxadiazin-2-one;

3-(4-(pyrimidine-2-carbonylamino)benzyl)-5-(3-methoxy-4-ethoxyphenyl)-6-ethyl-3,6-dihydro-1,3,4-oxadiazin-2-one;

3-(4-(pyrimidine-2-carbonylamino)benzyl)-5-(3-ethoxy-4-methoxyphenyl)-6-ethyl-3,6-dihydro-1,3,4-oxadiazin-2-one;

3-(4-(pyrimidine-2-carbonylamino)benzyl)-5-(3-hydroxy-4-methoxyphenyl)-6-ethyl-3,6-dihydro-1,3,4-oxadiazin-2-one;

3-(4-(pyrimidine-2-carbonylamino)benzyl)-5-(4-methylsulfonylphenyl)-6-ethyl-3,6-dihydro-1,3,4-oxadiazin-2-one;

3-(4-(pyrimidine-2-carbonylamino)benzyl)-5-(4-methyleneoxyphenyl)-6-ethyl-3,6-dihydro-1,3,4-oxadiazin-2-one;

3-(4-(pyrimidine-2-carbonylamino)benzyl)-5-(3-cyclopentyloxy-4-methoxyphenyl)-6-ethyl-3,6-dihydro-1,3,4-oxadiazin-2-one;

3-(3-(pyrimidine-2-carbonylamino)benzyl)-5-(3-cyclopentyloxy-4-methoxyphenyl)-6-ethyl-3,6-dihydro-1,3,4-oxadiazin-2-one;

3-(4-(pyrimidine-2-carbonylamino)phenethyl)-5-(3,4-dimethoxyphenyl)-3,6-dihydro-1,3,4-oxadiazin-2-one;

3-(4-(pyrimidine-2-carbonylamino)phenethyl)-5-(3,4-dimethoxyphenyl)-6-ethyl-3,6-dihydro-1,3,4-oxadiazin-2-one;

2-(4-(pyrazole-3-carbonylamino)benzyl)-6-(3,4-dimethoxyphenyl)-2,3,4,5-tetrahydropyridazin-3-one;

2-(3-(pyrazole-3-carbonylamino)benzyl)-6-(3,4-dimethoxyphenyl)-2,3,4,5-tetrahydropyridazin-3-one;

2-(2-(pyrazole-3-carbonylamino)benzyl)-6-(3,4-dimethoxyphenyl)-2,3,4,5-tetrahydropyridazin-3-one;

2-(4-(pyrazole-3-carbonylamino)benzyl)-6-(3,4-dimethoxyphenyl)-5-ethyl-2,3,4,5-tetrahydropyridazin-3-one;

2-(3-(pyrazole-3-carbonylamino)benzyl)-6-(3,4-dimethoxyphenyl)-5-ethyl-2,3,4,5-tetrahydropyridazin-3-one;

2-(2-(pyrazole-3-carbonylamino)benzyl)-6-(3,4-dimethoxyphenyl)-5-ethyl-2,3,4,5-tetrahydropyridazin-3-one;

2-(4-(pyrazole-3-carbonylamino)benzyl)-6-(3-methoxy-4-trifluoromethoxyphenyl)-5-ethyl-2,3,4,5-tetrahydropyridazin-3-one;

2-(4-(pyrazole-3-carbonylamino)benzyl)-6-(3-methoxy-4-difluoromethoxyphenyl)-5-ethyl-2,3,4,5-tetrahydropyridazin-3-one;

2-(4-(pyrazole-3-carbonylamino)benzyl)-6-(3-methoxy-4-fluoromethoxyphenyl)-5-ethyl-2,3,4,5-tetrahydropyridazin-3-one;

2-(4-(pyrazole-3-carbonylamino)benzyl)-6-(3-difluoromethoxy-4-methoxyphenyl)-5-ethyl-2,3,4,5-tetrahydropyridazin-3-one;

2-(4-(pyrazole-3-carbonylamino)benzyl)-6-(3-trifluoromethoxy-4-methoxyphenyl)-5-ethyl-2,3,4,5-tetrahydropyridazin-3-one;

2-(4-(pyrazole-3-carbonylamino)benzyl)-6-(3-fluoromethoxy-4-methoxyphenyl)-5-ethyl-2,3,4,5-tetrahydropyridazin-3-one;

2-(4-(pyrazole-3-carbonylamino)benzyl)-6-(3-methoxy-4-ethoxyphenyl)-5-ethyl-2,3,4,5-tetrahydropyridazin-3-one;

2-(4-(pyrazole-3-carbonylamino)benzyl)-6-(3-ethoxy-4-methoxyphenyl)-5-ethyl-2,3,4,5-tetrahydropyridazin-3-one;

2-(4-(pyrazole-3-carbonylamino)benzyl)-6-(3-hydroxy-4-methoxyphenyl)-5-ethyl-2,3,4,5-tetrahydropyridazin-3-one;

2-(4-(pyrazole-3-carbonylamino)benzyl)-6-(4-methylsulfonylphenyl)-5-ethyl-2,3,4,5-tetrahydropyridazin-3-one;

2-(4-(pyrazole-3-carbonylamino)benzyl)-6-(4-methylenoxyphenyl)-5-ethyl-2,3,4,5-tetrahydropyridazin-3-one;

2-(4-(pyrazole-3-carbonylamino)benzyl)-6-(3-cyclopentyloxy-4-methoxyphenyl)-5-ethyl-2,3,4,5-tetrahydropyridazin-3-one;

2-(3-(pyrazole-3-carbonylamino)benzyl)-6-(3-cyclopentyloxy-4-methoxyphenyl)-5-ethyl-2,3,4,5-tetrahydropyridazin-3-one;

2-(4-(pyrazole-3-carbonylamino)phenethyl)-6-(3,4-dimethoxyphenyl)-2,3,4,5-tetrahydropyridazin-3-one;

2-(4-(pyrazole-3-carbonylamino)phenethyl)-6-(3,4-dimethoxyphenyl)-5-ethyl-2,3,4,5-tetrahydropyridazin-3-one;

3-(4-(pyrazole-3-carbonylamino)benzyl)-5-(3,4-dimethoxyphenyl)-3,6-dihydro-1,3,4-thiadiazin-2-one;

3-(3-(pyrazole-3-carbonylamino)benzyl)-5-(3,4-dimethoxyphenyl)-3,6-dihydro-1,3,4-thiadiazin-2-one;

3-(2-(pyrazole-3-carbonylamino)benzyl)-5-(3,4-dimethoxyphenyl)-3,6-dihydro-1,3,4-thiadiazin-2-one;

3-(4-(pyrazole-3-carbonylamino)benzyl)-5-(3,4-dimethoxyphenyl)-6-ethyl-3,6-dihydro-1,3,4-thiadiazin-2-one;

3-(3-(pyrazole-3-carbonylamino)benzyl)-5-(3,4-dimethoxyphenyl)-6-ethyl-3,6-dihydro-1,3,4-thiadiazin-2-one;

3-(2-(pyrazole-3-carbonylamino)benzyl)-5-(3,4-dimethoxyphenyl)-6-ethyl-3,6-dihydro-1,3,4-thiadiazin-2-one;

3-(4-(pyrazole-3-carbonylamino)benzyl)-5-(3-methoxy-4-trifluoromethoxyphenyl)-6-ethyl-3,6-dihydro-1,3,4-thiadiazin-2-one;

3-(4-(pyrazole-3-carbonylamino)benzyl)-5-(3-methoxy-4-difluoromethoxyphenyl)-6-ethyl-3,6-dihydro-1,3,4-thiadiazin-2-one;

3-(4-(pyrazole-3-carbonylamino)benzyl)-5-(3-methoxy-4-fluoromethoxyphenyl)-6-ethyl-3,6-dihydro-1,3,4-thiadiazin-2-one;

3-(4-(pyrazole-3-carbonylamino)benzyl)-5-(3-difluoromethoxy-4-methoxyphenyl)-6-ethyl-3,6-dihydro-1,3,4-thiadiazin-2-one;

3-(4-(pyrazole-3-carbonylamino)benzyl)-5-(3-trifluoromethoxy-4-methoxyphenyl)-6-ethyl-3,6-dihydro-1,3,4-thiadiazin-2-one;

3-(4-(pyrazole-3-carbonylamino)benzyl)-5-(3-fluoromethoxy-4-methoxyphenyl)-6-ethyl-3,6-dihydro-1,3,4-thiadiazin-2-one;

3-(4-(pyrazole-3-carbonylamino)benzyl)-5-(3-methoxy-4-ethoxyphenyl)-5-ethyl-3,6-dihydro-1,3,4-thiadiazin-2-one;

3-(4-(pyrazole-3-carbonylamino)benzyl)-5-(3-ethoxy-4-methoxyphenyl)-6-ethyl-3,6-dihydro-1,3,4-thiadiazin-2-one;

3-(4-(pyrazole-3-carbonylamino)benzyl)-5-(3-hydroxy-4-methoxyphenyl)-6-ethyl-3,6-dihydro-1,3,4-thiadiazin-2-one;

3-(4-(pyrazole-3-carbonylamino)benzyl)-5-(4-methylsulfonylphenyl)-6-ethyl-3,6-dihydro-1,3,4-thiadiazin-2-one;

3-(4-(pyrazole-3-carbonylamino)benzyl)-5-(4-methyleneoxyphenyl)-6-ethyl-3,6-dihydro-1,3,4-thiadiazin-2-one;

3-(4-(pyrazole-3-carbonylamino)benzyl)-5-(3-cyclopentyloxy-4-methoxyphenyl)-6-ethyl-3,6-dihydro-1,3,4-thiadiazin-2-one;

3-(3-(pyrazole-3-carbonylamino)benzyl)-5-(3-cyclopentyloxy-4-methoxyphenyl)-6-ethyl-3,6-dihydro-1,3,4-thiadiazin-2-one;

3-(4-(pyrazole-3-carbonylamino)phenethyl)-5-(3,4-dimethoxyphenyl)-3,6-dihydro-1,3,4-thiadiazin-2-one;

3-(4-(pyrazole-3-carbonylamino)phenethyl)-5-(3,4-dimethoxyphenyl)-6-ethyl-3,6-dihydro-1,3,4-thiadiazin-2-one;

3-(4-(pyrazole-3-carbonylamino)benzyl)-5-(3,4-dimethoxyphenyl)-3,6-dihydro-1,3,4-oxadiazin-2-one;

3-(3-(pyrazole-3-carbonylamino)benzyl)-5-(3,4-dimethoxyphenyl)-3,6-dihydro-1,3,4-oxadiazin-2-one;

3-(2-(pyrazole-3-carbonylamino)benzyl)-5-(3,4-dimethoxyphenyl)-3,6-dihydro-1,3,4-oxadiazin-2-one;

3-(4-(pyrazole-3-carbonylamino)benzyl)-5-(3,4-dimethoxyphenyl)-6-ethyl-3,6-dihydro-1,3,4-oxadiazin-2-one;

3-(3-(pyrazole-3-carbonylamino)benzyl)-5-(3,4-dimethoxyphenyl)-6-ethyl-3,6-dihydro-1,3,4-oxadiazin-2-one;

3-(2-(pyrazole-3-carbonylamino)benzyl)-5-(3,4-dimethoxyphenyl)-6-ethyl-3,6-dihydro-1,3,4-oxadiazin-2-one;

3-(4-(pyrazole-3-carbonylamino)benzyl)-5-(3-methoxy-4-trifluoromethoxyphenyl)-6-ethyl-3,6-dihydro-1,3,4-oxadiazin-2-one;

3-(4-(pyrazole-3-carbonylamino)benzyl)-5-(3-methoxy-4-difluoromethoxyphenyl)-6-ethyl-3,5-dihydro-1,3,4-oxadiazin-2-one;

3-(4-(pyrazole-3-carbonylamino)benzyl)-5-(3-methoxy-4-fluoromethoxyphenyl)-6-ethyl-3,6-dihydro-1,3,4-oxadiazin-2-one;

3-(4-(pyrazole-3-carbonylamino)benzyl)-5-(3-difluoromethoxy-4-methoxyphenyl)-6-ethyl-3,6-dihydro-1,3,4-oxadiazin-2-one;

3-(4-(pyrazole-3-carbonylamino)benzyl)-5-(3-trifluoromethoxy-4-methoxyphenyl)-6-ethyl-3,6-dihydro-1,3,4-oxadiazin-2-one;

3-(4-(pyrazole-3-carbonylamino)benzyl)-5-(3-fluoromethoxy-4-methoxyphenyl)-6-ethyl-3,6-dihydro-1,3,4-oxadiazin-2-one;

3-(4-(pyrazole-3-carbonylamino)benzyl)-5-(3-methoxy-4-ethoxyphenyl)-6-ethyl-3,6-dihydro-1,3,4-oxadiazin-2-one;

3-(4-(pyrazole-3-carbonylamino)benzyl)-5-(3-ethoxy-4-methoxyphenyl)-6-ethyl-3,6-dihydro-1,3,4-oxadiazin-2-one;

3-(4-(pyrazole-3-carbonylamino)benzyl)-5-(3-hydroxy-4-methoxyphenyl)-6-ethyl-3,6-dihydro-1,3,4-oxadiazin-2-one;

3-(4-(pyrazole-3-carbonylamino)benzyl)-5-(4-methylsulfonylphenyl)-6-ethyl-3,6-dihydro-1,3,4-oxadiazin-2-one;

3-(4-(pyrazole-3-carbonylamino)benzyl)-5-(4-methyleneoxyphenyl)-6-ethyl-3,6-dihydro-1,3,4-oxadiazin-2-one;

3-(4-(pyrazole-3-carbonylamino)benzyl)-5-(3-cyclopentyloxy-4-methoxyphenyl)-6-ethyl-3,6-dihydro-1,3,4-oxadiazin-2-one;

3-(3-(pyrazole-3-carbonylamino)benzyl)-5-(3-cyclopentyloxy-4-methoxyphenyl)-6-ethyl-3,6-dihydro-1,3,4-oxadiazin-2-one;

3-(4-(pyrazole-3-carbonylamino)phenethyl)-5-(3,4-dimethoxyphenyl)-3,6-dihydro-1,3,4-oxadiazin-2-one;

3-(4-(pyrazole-3-carbonylamino)phenethyl)-5-(3,4-dimethoxyphenyl)-6-ethyl-3,6-dihydro-1,3,4-oxadiazin-2-one;

2-(3-nicotinoylaminobenzyl)-6-(3-ethoxy-4-methoxyphenyl)-2,3,4,5-tetrahydropyridazin-3-one, m.p. 204°;

2-(4-isoncotinoylaminobenzyl)-6-(3-ethoxy-4-methoxyphenyl)-2,3,4,5-tetrahydropyridazin-3-one, hydrochloride m.p. 252°;

2-(4-pyrazinecarbonylaminobenzyl)-6-(3-ethoxy-4-methoxyphenyl)-2,3,4,5-tetrahydropyridazin-3-one, m.p. 202°;

2-(4-(isoxazole-5-carbonylamino)benzyl)-6-(3-ethoxy-4-methoxyphenyl)-2,3,4,5-tetrahydropyridazin-3-one, m.p. 192°;

2-(4-nicotinoylaminobenzyl)-6-(3-cyclopentyloxy-4-methoxyphenyl)-2,3,4,5-tetrahydropyridazin-3-one, m.p. 205°

2-(4-nicotinoylaminobenzyl)-6-(3,4-dimethoxyphenyl)-2,3,4,5-tetrahydropyridazin-3-one, hydrochloride m.p. 212°;

d) compounds, disclosed in EP 0 539 806 b1, selected from the group consisting of

5-(3-methoxy-4-difluoromethoxyphenyl)-6-ethyl-3,6-dihydro-1,3,4-thiadiazin-2-one, m.p. 97°;

5-(3-methoxy-4-trifluoromethoxyphenyl)-6-ethyl-3,6-dihydro-1,3,4-thiadiazin-2-one;

5-(3-methoxy-4-trifluoromethoxyphenyl)-6-methyl-3,6-dihydro-1,3,4-thiadiazin-2-one;

5-(3-methoxy-4-difluoromethoxyphenyl)-6-methyl-3,6-dihydro-1,3,4-thiadiazin-2-one;

5-[3-methoxy-4-(1,1,2,2-tetrafluoroethoxy)phenyl]-6-ethyl-3,6-dihydro-1,3,4-thiadiazin-2-one;

5-(3-methoxy-4-chloromethoxyphenyl)-6-ethyl-3,6-dihydro-1,3,4-thiadiazin-2-one;

5-(3-methoxy-4-chloromethoxyphenyl)-6-methyl-3,6-dihydro-1,3,4-thiadiazin-2-one;

5-(3-methoxy-4-pentachloroethoxyphenyl)-6-ethyl-3,6-dihydro-1,3,4-thiadiazin-2-one;

5-(3-methoxy-4-trifluoromethoxyphenyl)-6-propyl-3,6-dihydro-1,3,4-thiadiazin-2-one;

5-(3-methoxy-4-difluoromethoxyphenyl)-6-propyl-3,6-dihydro-1,3,4-thiadiazin-2-one;

5-[3-methoxy-4-(1,1,2,-trifluoroethoxy)phenyl]-6-ethyl-3,6-dihydro-1,3,4-thiadiazin-2-one;

5-[3-methoxy-4-(1,1,2,-trifluoroethoxy)phenyl]-6-methyl-3,6-dihydro-1,3,4-thiadiazin-2-one;

5-(3-methoxy-4-difluoromethoxyphenyl)-3,6-dihydro-1,3,4-thiadiazin-2-one, m.p. 120°;

5-(3-methoxy-4-trifluoromethoxyphenyl)-3,6-dihydro-1,3,4-thiadiazin-2-one;

5-(4-trifluoromethoxyphenyl)-3,6-dihydro-1,3,4-thiadiazin-2-one;

5-[3-methoxy-4-(1,1,2,2-tetrafluoroethoxy)phenyl]-3,6-dihydro-1,3,4-thiadiazin-2-one;

5-(3-methoxy-4-chloromethoxyphenyl)-3,6-dihydro-1,3,4-thiadiazin-2-one;

5-(3-methoxy-4-dichloromethoxyphenyl)-3,6-dihydro-1,3,4-thiadiazin-2-one;

5-(3-methoxy-4-pentachloroethoxyphenyl)-3,6-dihydro-1,3,4-thiadiazin-2-one;

5-(4-difluoromethoxyphenyl)-3,6-dihydro-1,3,4-thiadiazin-2-one;

5-[3-methoxy-4-(1,1,2,2,3-pentafluoropropoxy)phenyl]-6-ethyl-3,6-dihydro-1,3,4-thiadiazin-2-one;

5-[bis-3,4-(difluoromethoxy)phenyl]-3,6-dihydro-1,3,4-thiadiazin-2-one;

5-[bis-3,4-(dichloromethoxy)phenyl]-3,6-dihydro-1,3,4-thiadiazin-2-one;

5-[bis-3,4-(1,2-difluoroethoxy)phenyl]-3,6-dihydro-1,3,4-thiadiazin-2-one;

5-[3-ethoxy-4-(1,1,2,2,-tetrafluoroethoxy)phenyl]-3,6-dihydro-1,3,4-thiadiazin-2-one;

5-[3-methoxy-4-(1,2,2,-trichloroethoxy)phenyl]-3,6-dihydro-1,3,4-thiadiazin-2-one;

5-[4-(2,2,2-trifluoroethoxy)phenyl]-6-ethyl-3,6-dihydro-1,3,4-thiadiazin-2-one, m.p. 102°;

5-[3-methoxy-4-(2,2,2-trifluoroethoxy)phenyl]-6-ethyl-3,6-dihydro-1,3,4-thiadiazin-2-one, m.p. 123-125°;

5-[3-methoxy-4-(2,2,2-trifluoroethoxy)phenyl]-3,6-dihydro-1,3,4-thiadiazin-2-one, m.p. 120°;

5-[3-(2,2,2-trifluoroethoxy)-4-methoxyphenyl]-6-ethyl-3,6-dihydro-1,3,4-thiadiazin-2-one, m.p. 120-121°;

5-(3-difluoromethoxy-4-methoxyphenyl)-6-ethyl-3,6-dihydro-1,3,4-thiadiazin-2-one, m.p. 105°;

e) compounds, disclosed in EP 0 618 201 A1, selected from the group consisting of

3-dimethylaminopropyl-5-(3,4-dimethoxyphenyl)-6-ethyl-3,6-dihydro-1,3,4-thiadiazinon-2-one, m.p. 175°;

3-dimethylaminopropyl-5-(3-methoxy-4-trifluoromethoxyphenyl)-6-ethyl-3,6-dihydro-1,3,4-thiadiazinon-2-one;

3-dimethylaminopropyl-5-(3-methoxy-4-difluoromethoxyphenyl)-6-ethyl-3,6-dihydro-1,3,4-thiadiazinon-2-one;

3-dimethylaminopropyl-5-(3-methoxy-4-fluoromethoxyphenyl)-6-ethyl-3,6-dihydro-1,3,4-thiadiazinon-2-one;

3-dimethylaminopropyl-5-(4-methoxy-3-difluoromethoxyphenyl)-6-ethyl-3,6-dihydro-1,3,4-thiadiazinon-2-one;

3-dimethylaminopropyl-5-[4-methoxy-3-(2,2,2-trifluoroethoxy)phenyl]-6-ethyl-3,6-dihydro-1,3,4-thiadiazinon-2-one;

3-dimethylaminopropyl-5-(4-methoxy-3-fluoromethoxyphenyl)-6-ethyl-3,6-dihydro-1,3,4-thiadiazinon-2-one;

3-dimethylaminopropyl-5-(3-methoxy-4-ethoxyphenyl)-6-ethyl-3,6-dihydro-1,3,4-thiadiazinon-2-one;

3-dimethylaminopropyl-5-(4-methoxy-3-ethoxyphenyl)-6-ethyl-3,6-dihydro-1,3,4-thiadiazinon-2-one;

3-dimethylaminopropyl-5-(3-methoxy-4-hydroxyphenyl)-6-ethyl-3,6-dihydro-1,3,4-thiadiazinon-2one;

3-dimethylaminopropyl-5-(3,4-dimethoxyphenyl)-3,6dihydro-1,3,4-thiadiazinon-2-one;

2-dimethylaminoethyl-5-(3,4-dimethoxyphenyl)-6-ethyl-3,6-dihydro-1,3,4-thiadiazinon-2-one;

2-dimethylaminoethyl-5-(3-methoxy-4-trifluoromethoxyphenyl)-6-ethyl-3,6-dihydro-1,3,4-thiadiazinon-2-one;

2-dimethylaminoethyl-5-(3-methoxy-4-difluoromethoxyphenyl)-6-ethyl-3,6-dihydro-1,3,4-thiadiazinon-2-one;

2-dimethylaminoethyl-5-(3-methoxy-4-fluoromethoxyphenyl)-6-ethyl-3,6-dihydro-1,3,4-thiadiazinon-2-one;

2-dimethylaminoethyl-5-(4-methoxy-3-difluoromethoxyphenyl)-6-ethyl-3,6-dihydro-1,3,4-thiadiazinon-2-one;

2-dimethylaminoethyl-5-(4-methoxy-3-fluoromethoxyphenyl)-6-ethyl-3,6-dihydro-1,3,4-thiadiazinon-2-one;

2-dimethylaminoethyl-5-(3-methoxy-4-ethoxyphenyl)-6-ethyl-3,6-dihydro-1,3,4-thiadiazinon-2-one;

2-dimethylaminoethyl-5-(4-methoxy-3-ethoxyphenyl)-6-ethyl-3,6-dihydro-1,3,4-thiadiazinon-2-one;

2-dimethylaminoethyl-5-(4-methoxy-3-hydroxyphenyl)-6-ethyl-3,6-dihydro-1,3,4-thiadiazinon-2-one;

3-morpholinopropyl-5-[3-methoxy-4-(1,1,2,2,3-pentafluoropropoxy)phenyl]-6-ethyl-3,6-dihydro-1,3,4-thiadiazinon-2-one;

3-dimethylaminopropyl-5-[3,4,-bis-(difluoromethoxy)phenyl]-3,6-dihydro-1,3,4-thiadiazinon-2-one;

3-dimethylaminopropyl-5-[3-methoxy-4-(1,1,2-trifluoroethoxy)phenyl]-3,6-dihydro-1,3,4-thiadiazinon-2-one;

3-dimethylaminopropyl-5-[3,4-bis-(chloromethoxy)phenyl]-3,6-dihydro-1,3,4-thiadiazinon-2-one;

3-morpholinopropyl-5-(3-methoxy-4-fluoromethoxyphenyl)-6-ethyl-3,6-dihydro-1,3,4-thiadiazinon-2-one;

3-morpholinopropyl-5-(3-methoxy-4-trifluoromethoxyphenyl)-6-ethyl-3,6-dihydro-1,3,4-thiadiazinon-2-one;

3-piperidinopropyl-5-(3-methoxy-4-difluoromethoxyphenyl)-6-ethyl-3,6-dihydro-1,3,4-thiadiazinon-2-one;

3-morpholinopropyl-5-(3,4-dimethoxyphenyl)-6-ethyl-3,6-dihydro-1,3,4-thiadiazinon-2-one;

3-piperidinopropyl-5-(3,4-dimethoxyphenyl))-6-ethyl-3,6-dihydro-1,3,4-thiadiazinon-2-one;

3-pyrrolidinopropyl-5-(3,4-dimethoxyphenyl)-6-ethyl-3,6-dihydro-1,3,4-thiadiazinon-2-one;

3-morpholinopropyl-5-(3-methoxy-4-ethoxyphenyl)-6-ethyl-3,6-dihydro-1,3,4-thiadiazinon-2-one;

3-piperidinopropyl-5-(3-methoxy-4-ethoxyphenyl)-6-ethyl-3,6-dihydro-1,3,4-thiadiazinon-2-one;

3-pyrrolidinopropyl-5-(3-methoxy-4-ethoxyphenyl)-6-ethyl-3,6-dihydro-1,3,4-thiadiazinon-2-one;

3-morpholinopropyl-5-(4-methoxy-3-ethoxyphenyl)-6-ethyl-3,6-dihydro-1,3,4-thiadiazinon-2-one;

3-piperidinopropyl-5-(4-methoxy-3-ethoxyphenyl)-6-ethyl-3,6-dihydro-1,3,4-thiadiazinon-2-one;

3-morpholinopropyl-5-(3-methoxy-4-difluoromethoxyphenyl)-6-ethyl-3,6-dihydro-1,3,4-thiadiazinon-2-one;

3-piperidinopropyl-5-(4-methoxy-3-difluoromethoxyphenyl)-6-ethyl-3,6-dihydro-1,3,4-thiadiazinon-2-one;

3-piperidinopropyl-5-[3-(2,2,2-trifluoroethoxy)-4-methoxyphenyl]-6-ethyl-3,6-dihydro-1,3,4-thiadiazinon-2-one;

3-morpholinopropyl-5-[3-(2,2,2-trifluoroethoxy)-4-methoxyphenyl]-6-ethyl-3,6-dihydro-1,3,4-thiadiazinon-2-one;

2-morpholinoethyl-5-(3-methoxy-4-fluoromethoxyphenyl)-6-ethyl-3,6-dihydro-1,3,4-thiadiazinon-2-one;

2-morpholinoethyl-5-(3-methoxy-4-trifluoromethoxyphenyl)-6-ethyl-3,6-dihydro-1,3,4-thiadiazinon-2-one;

f) Compounds, disclosed in DE 196 04 388 A1, selected from the group consisting of

3-(N,N-diethylamino)propyl N-[4-(3,6-dihydro-5-(3,4-dimethoxyphenyl)-2-oxo-2H-1,3,4-thiadiazin-3-ylmethyl)phenyl]carbamate;

2-(N,N-diethylamino)ethyl N-[4-(3,6-dihydro-5-(3,4-dimethoxyphenyl)-2-oxo-2H-1,3,4-thiadiazin-3-ylmethyl)phenyl]carbamate;

2-(N,N-diisopropylamino)ethyl N-[4-(3,6-dihydro-5-(3,4-dimethoxyphenyl)-2-oxo-2H-1,3,4-thiadiazin-3-ylmethyl)phenyl]carbamate, hydrochloride, m.p. 218°;

6-(N,N-dimethylamino)hexyl N-[4-(3,6-dihydro-5-(3,4-dimethoxyphenyl)-2-oxo-2H-1,3,4-thiadiazin-3-ylmethylphenyl]carbamate;

2-(N,N-dimethylamino)-1-methylethyl N-[4-(3,6-dihydro-5-(3,4-dimethoxyphenyl)-2-oxo-2H-1,3,4-thiadiazin-3-ylmethyl)phenyl]carbamate;

1-methyl-4-piperidyl N-[4-(3,6-dihydro-5-(3,4-dimethoxyphenyl)-2-oxo-2H-1,3,4-thiadiazin-3-ylmethyl)phenyl]carbamate, m.p. 183°;

(4-hydroxy-1-piperidyl)ethyl N-[-4-(3,6-dihydro-5-(3,4-dimethoxyphenyl)-2-oxo-2H-1,3,4-thiadiazin-3-ylmethyl)phenyl]carbamate;

2-morpholinoethyl N-[4-(3,6-dihydro-5-(3,4-dimethoxyphenyl)-2-oxo-2H-1,3,4-thiadiazin-3-ylmethyl)phenyl]carbamate;

3-morpholinopropyl N-[4-(3,5-dihydro-5-(3,4-dimethoxyphenyl)-2-oxo-2H-1,3,4-thiadiazin-3-ylmethyl)phenyl]carbamate;

2-thiomorpholinoethyl N-[4-(3,6-dihydro-5-(3,4-dimethoxyphenyl)-2-oxo-2H-1,3,4-thiadiazin-3-ylmethyl)phenyl]carbamate;

3-thiomorpholinopropyl N-[4-(3,6-dihydro-5-(3,4-dimethoxyphenyl)-2-oxo-2H-1,3,4-thiadiazin -3-ylmethylphenyl]carbamate;

2-piperazinoethyl N-[4-(3,6-dihydro-5-(3,4-dimethoxyphenyl)-2-oxo-2H-1,3,4-thiadiazin-3-ylmethyl)phenyl]carbamate;

3-piperazinopropyl N-[4-(3,6-dihydro-5-(3,4-dimethoxyphenyl)-2-oxo-2H-1,3,4-thiadiazin-3-ylmethyl)phenyl]carbamate;

2-pyrrolidinoethyl N-[4-(3,6-dihydro-5-(3,4-dimethoxyphenyl)-2-oxo-2H-1,3,4-thiadiazin-3-ylmethyl)phenyl]carbamate;

3-pyrrolidinopropyl N-[4-(3,6-dihydro-5-(3,4-dimethoxyphenyl)-2-oxo-2H-1,3,4-thiadiazin-3-ylmethyl)phenyl]carbamate;

3-(N,N-diethylamino)propyl N-[3-(3,6-dihydro-5-(3,4-dimethoxyphenyl)-2-oxo-2H-1,3,4-thiadiazin-3-ylmethyl)phenyl]carbamate, hydrochloride, m,p. 161°;

2-(N,N-diethylamino)ethyl N-[3-(3,6-dihydro-5-(3,4-dimethoxyphenyl)-2-oxo-2H-1,3,4-thiadiazin-3-ylmethyl)phenyl]carbamate;

2-(N,N-diisopropylamino)ethyl N-[3-(3,6-dihydro-5-(3,4-dimethoxyphenyl)-2-oxo-2H-1,3,4-thiadiazin-3-ylmethyl)phenyl]carbamate;

6-(N,N-dimethylamino)hexyl N-[3-(3,6-dihydro-5-(3,4-dimethoxyphenyl)-2-oxo-2H-1,3,4-thiadiazin-3-ylmethyl)phenyl]carbamate;

2-(N,N-dimethylamino)-1-methylethyl N-[3-(3,6-dihydro-5-(3,4-dimethoxyphenyl)-2-oxo-2H-1,3,4-thiadiazin-3-ylmethyl)phenyl]carbamate;

1-methyl-4-piperidyl N-[3-(3,6-dihydro-5-(3,4-dimethoxyphenyl)-2-oxo-2H-1,3,4-thiadiazin-3-ylmethyl)phenyl]carbamate;

2-morpholinoethyl N-[3-(3,6-dihydro-5-(3,4-dimethoxyphenyl)-2-oxo-2H-1,3,4-thiadiazin-3-ylmethyl)phenyl]carbamate;

3-morpholinopropyl N-[3-(3,6-dihydro-5-(3,4-dimethoxyphenyl)-2-oxo-2H-1,3,4-thiadiazin-3-ylmethyl)phenyl]carbamate;

2-thiomorpholinoethyl N-[3-(3,6-dihydro-5-(3,4-dimethoxyphenyl)-2-oxo-2-1,3,4-thiadiazin-3-ylmethyl)phenyl]carbamate;

3-thiomorpholinopropyl N-[3-(3,6-dihydro-5-(3,4-dimethoxyphenyl)-2-oxo-2H-1,3,4-thiadiazin-3-ylmethyl)phenyl]carbamate;

2-piperazinoethyl N-[3-(3,6-dihydro-5-(3,4-dimethoxyphenyl)-2-oxo-2H -1,3,4-thiadiazin-3-ylmethyl)phenyl]carbamate;

3-piperazinopropyl N-[3-(3,6-dihydro-5-(3,4-dimethoxyphenyl)-2-oxo-2H-1,3,4-thiadiazin-3-ylmethyl)phenyl]carbamate;

2-pyrrolidinoethyl N-[3-(3,6-dihydro-5-(3,4-dimethoxyphenyl)-2-oxo-2H-1,3,4-thiadiazin-3-ylmethyl)phenyl]carbamate;

3-pyrrolidinopropyl N-[3-(3,6-dihydro-5-(3,4-dimethoxyphenyl)-2-oxo-2H-1,3,4-thiadiazin-3-ylmethyl)phenyl]carbamate;

3-(N,N-diethylamino)propyl N-[4-(3,6-dihydro-5-(3-ethoxy-4-methoxyphenyl)-2-oxo-2H-1,3,4-thiadiazin-3-ylmethyl)phenyl]carbamate;

2-(N,N-diethylamino)ethyl N-[4-(3,6-dihydro-5-(3-ethoxy-4-methoxyphenyl)-2-oxo-2H-1,3,4-thiadiazin-3-ylmethyl)phenyl]carbamate;

2-(N,N-diisopropylamino)ethyl N-[4-(3,6-dihydro-5-(3-ethoxy-4-methoxyphenyl)-2-oxo-2H-1,3,4-thiadiazin-3-ylmethyl)phenyl]carbamate;

6-(N,N-dimethylamino)hexyl N-[4-(3,6-dihydro-5-(3-ethoxy-4-methoxyphenyl)-2-oxo-2H-1,3,4-thiadiazin-3-ylmethyl)phenyl]carbamate;

2-(N,N-dimethylamino)-1-methylethyl N-[4-(3,6-dihydro-5-(3-ethoxy-4-methoxyphenyl)-2-oxo-2H-1,3,4-thiadiazin-3-ylmethyl)phenyl]carbamate;

1-methyl-4-piperidyl N-[4-(3,6-dihydro-5-(3-ethoxy-4-methoxyphenyl)-2-oxo-2H-1,3,4-thiadiazin-3-ylmethyl)phenyl]carbamate, m.p. 153;

2-morpholinoethyl N-[4-(3,6-dihydro-5-(3-ethoxy-4-methoxyphenyl)-2-oxo-2H-1,3,4-thiadiazin-3-ylmethyl)phenyl]carbamate;

3-morpholinopropyl N-[4-(3,6-dihydro-5-(3-ethoxy-4-methoxyphenyl)-2-oxo-2H-1,3,4-thiadiazin-3-ylmethyl)phenyl]carbamate;

2-thiomorpholinoethyl N-[4-(3,6-dihydro-5-(3-ethoxy-4-methoxyphenyl)-2-oxo-2H-1,3,4-thiadiazin-3-ylmethyl)phenyl]carbamate;

3-thiomorpholinopropyl N-[4-(3,6-dihydro-5-(3-ethoxy-4-methoxyphenyl)-2-oxo-2H-1,3,4-thiadiazin-3-ylmethyl)phenyl]carbamate;

2-piperazinoethyl N-[4-(3,6-dihydro-5-(3-ethoxy-4-methoxyphenyl)-2-oxo-2H-1,3,4-thiadiazin-3ylmethyl)phenyl]carbamate;

3-piperazinopropyl N-[4-(3,6-dihydro-5-(3-ethoxy-4-methoxyphenyl)-2-oxo-2H-1,3,4-thiadiazin-3-ylmethyl)phenyl]carbamate;

2-pyrrolidinoethyl N-[4-(3,6-dihydro-5-(3-ethoxy-4-methoxyphenyl)-2-oxo-2H-1,3,4-thiadiazin-3-ylmethyl)phenyl]carbamate;

3-pyrrolidinopropyl N-[4-(3,6-dihydro-5-(3-ethoxy-4-methoxyphenyl)-2-oxo-2H-1,3,4-thiadiazin-3-ylmethyl)phenyl]carbamate;

3-(N,N-diethylamino)propyl N-[4-(3,6-dihydro-5-(3,4-dimethoxyphenyl)-6-ethyl-2-oxo-2H-1,3,4-thiadiazin-3-ylmethyl)phenyl]carbamate;

2-(N,N-diethylamino)ethyl N-[4-(3,6-dihydro-5-(3,4-dimethoxyphenyl)-6-ethyl-2-oxo-2H-1,3,4-thiadiazin-3-ylmethyl)phenyl]carbamate;

2-(N,N-diisopropylamino)ethyl N-[4-(3,6-dihydro-5-(3,4-dimethoxyphenyl)-6-ethyl-2-oxo-2H-1,3,4-thiadiazin-3-ylmethyl)phenyl]carbamate;

6-(N,N-dimethylamino)hexyl N-[4-(3,6-dihydro-5-(3,4-dimethoxyphenyl)-6-ethyl-2-oxo-2H-1,3,4-thiadiazin-3-ylmethyl)phenyl]carbamate;

2-(N,N-dimethylamino)-1-methylethyl N-[4-(3,6-dihydro-5-(3,4-dimethoxyphenyl)-6-ethyl-2-oxo-2H-1,3,4-thiadiazin-3-ylmethyl)phenyl]carbamate;

1-methyl-4-piperidyl N-[4-(3,6-dihydro-5-(3,4-dimethoxyphenyl)-6-ethyl-2-oxo-2H-1,3,4-thiadiazin-3-ylmethyl)phenyl]carbamate;

2-morpholinoethyl N-[4-(3,6-dihydro-5-(3,4-dimethoxyphenyl)-6-ethyl-2-oxo-2H-1,3,4-thiadiazin-3-ylmethyl)phenyl]carbamate;

3-morpholinopropyl N-[4-(3,6-dihydro-5-(3,4-dimethoxyphenyl)-6-ethyl-2-oxo-2H-1,3,4-thiadiazin-3-ylmethyl)phenyl]carbamate;

2-thiomorpholinoethyl N-[4-(3,6-dihydro-5-(3,4-dimethoxyphenyl)-6-ethyl-2-oxo-2H-1,3,4-thiadiazin-3-ylmethyl)phenyl]carbamate;

3-thiomorpholinopropyl N-[4-(3,6-dihydro-5-(3,4-dimethoxyphenyl)-6-ethyl-2-oxo-2H-1,3,4-thiadiazin-3-ylmethyl)phenyl]carbamate;

2-piperazinoethyl N-[4-(3,6-dihydro-5-(3,4dimethoxyphenyl)-6-ethyl-2-oxo-2H-1,3,4-thiadiazin-3-ylmethyl)phenyl]carbamate;

3-piperazinopropyl N-[4-(3,6-dihydro-5-(3,4-dimethoxyphenyl)-6-ethyl-2-oxo-2H-1,3,4-thiadiazin-3-ylmethyl)phenyl]carbamate;

2-pyrrolidinoethyl N-[4-(3,6-dihydro-5-(3,4-dimethoxyphenyl)-6-ethyl-2-oxo-2H-1,3,4-thiadiazin-3-ylmethyl)phenyl]carbamate;

3-pyrrolidinopropyl N-[4-(3,6-dihydro-5-(3,4-dimethoxyphenyl)-6-ethyl-2-oxo-2H-1,3,4-thiadiazin-3-ylmethyl)phenyl]carbamate;

3-(N,N-diethylamino)propyl N-[4-(3,6-dihydro-5-(3-ethoxy-4-methoxyphenyl)-6-ethyl-2oxo-2H-1,3,4-thiadiazin-3-ylmethyl)phenyl]carbamate;

2-(N,N-diethylamino)ethyl N-[4-(3,6-dihydro-5-(3-ethoxy-4-methoxyphenyl)-6-ethyl-2-oxo-2H-1,3,4-thiadiazin-3-ylmethyl)phenyl]carbamate;

2-(N,N-diisopropylamino)ethyl N-[4-(3,6-dihydro-5-(3-ethoxy-4-methoxyphenyl)-6-ethyl-2-oxo-2H-1,3,4-thiadiazin-3-ylmethyl)phenyl]carbamate;

6-(N,N-dimethylamino)hexyl N-[4-(3,6-dihydro-5-(3-ethoxy-4-methoxyphenyl)-6-ethyl-2-oxo-2H-1,3,4-thiadiazin-3-ylmethyl)phenyl]carbamate;

2-(N,N-dimethylamino)-1-methylethyl N-[4-(3,6-dihydro-5-(3-ethoxy-4-methoxyphenyl)-6-ethyl-2-oxo-2H-1,3,4-thiadiazin-3-ylmethyl)phenyl]carbamate;

1-methyl-4-piperidyl N-[4-(3,6-dihydro-5-(3-ethoxy-4-methoxyphenyl)-6-ethyl-2-oxo-2H-1,3,4-thiadiazin-3-ylmethyl)phenyl]carbamate;

2-morpholinoethyl N-[4-(3,6-dihydro-5-(3-ethoxy-4-methoxyphenyl)-6-ethyl-2-oxo-2H-1,3,4-thiadiazin-3-ylmethyl)phenyl]carbamate;

3-morpholinopropyl-2-oxo-2H-1,3,4-thiadiazin-3-ylmethyl)phenyl]carbamate;

2-thiomorpholinoethyl N-[4-(3,6-dihydro-5-(3-ethoxy-4-methoxyphenyl)-6-ethyl-2-oxo-2H-1,3,4-thiadiazin-3-ylmethyl)phenyl]carbamate;

3-thiomorpholinopropyl N-[4-(3,6-dihydro-5-(3-ethoxy-4-methoxyphenyl)-6-ethyl-2-oxo-2H-1,3,4-thiadiazin-3-ylmethyl)phenyl]carbamate;

2-piperazinoethyl N-[4-(3,6-dihydro-5-(3-ethoxy-4-methoxyphenyl)-6-ethyl-2-oxo-2H-1,3,4-thiadiazin-3-ylmethyl)phenyl]carbamate;

3-piperazinopropyl N-[4-(3,6-dihydro-5-(3-ethoxy-4-methoxyphenyl)-6-ethyl-2-oxo-2H-1,3,4-thiadiazin-3-ylmethyl)phenyl]carbamate;

2-pyrrolidinoethyl N-[4-(3,6-dihydro-5-(3-ethoxy-4-methoxyphenyl)-6-ethyl-2-oxo-2H-1,3,4-thiadiazin-3-ylmethyl)phenyl]carbamate;

3-pyrrolidinopropyl N-[4-(3,6-dihydro-5-(3-ethoxy-4-methoxyphenyl)-6-ethyl-2-oxo-2H-1,3,4-thiadiazin-3-ylmethyl)phenyl]carbamate;

3-(N,N-diethylamino)propyl N-[4-(3,6-dihydro-5-(3-cyclopentyloxy-4-methoxyphenyl)-2-oxo-2H-1,3,4-thiadiazin-3-ylmethyl)phenyl]carbamate;

2-(N,N-diethylamino)ethyl N-[4-(3,6-dihydro-5-(3-cyclopentyloxy-4-methoxyphenyl)-2-oxo-2H-1,3,4-thiadiazin-3-ylmethyl)phenyl]carbamate;

2-(N,N-diisopropylamino)ethyl N-[4-(3,6-dihydro-5-(3-cyclopentyloxy-4-methoxyphenyl)-2oxo-2H-1,3,4-thiadiazin-3-ylmethyl)phenyl]carbamate;

6-(N,N-dimethylamino)hexyl N-[4-(3,6-dihydro-5-(3-cyclopentyloxy-4-methoxyphenyl)-2-oxo-2H-1,3,4-thiadiazin-3-ylmethyl)phenyl]carbamate;

2-(N,N-dimethylamino)-1-methylethyl N-[4-(3,6-dihydro-5-(3-cyclopentyloxy-4-methoxyphenyl)-2-oxo-2H-1,3,4-thiadiazin-3-ylmethyl)phenyl]carbamate;

1-methyl-4-piperidyl N-[4-(3,6-dihydro-5-(3-cyclopentyloxy-4-methoxyphenyl)-2-oxo-2H-1,3,4-thiadiazin-3-ylmethyl)phenyl]carbamate;

2-morpholinoethyl N-[4-(3,6-dihydro-5-(3-cyclopentyloxy-4-methoxyphenyl)-2-oxo-2H-1,3,4-thiadiazin-3-ylmethyl)phenyl]carbamate;

3-morpholinopropyl N-[4-(3,6-dihydro-5-(3-cyclopentyloxy-4-methoxyphenyl)-2-oxo-2H-1,3,4-thiadiazin-3-ylmethyl)phenyl]carbamate;

2-thiomorpholinoethyl N-[4-(3,6-dihydro-5-(3-cyclopentyloxy-4-methoxyphenyl)-2-oxo-2H-1,3,4-thiadiazin-3-ylmethyl)phenyl]carbamate;

3-thiomorpholinopropyl N-[4-(3,6-dihydro-5-(3-cyclopentyloxy-4-methoxyphenyl)-2-oxo-2H-1,3,4-thiadiazin-3-ylmethyl)phenyl]carbamate;

2-piperazinoethyl N-[4-(3,6-dihydro-5-(3-cyclopentyloxy-4-methoxyphenyl)-2-oxo-2H-1,3,4-thiadiazin-3-ylmethyl)phenyl]carbamate;

3-piperazinopropyl N-[4-(3,6-dihydro-5-(3-cyclopentyloxy-4-methoxyphenyl)-2-oxo-2H-1,3,4-thiadiazin-3-ylmethyl)phenyl]carbamate;

2-pyrrolidinoethyl N-[4-(3,6-dihydro-5-(3-cyclopentyloxy-4-methoxyphenyl)-2-oxo-2H-1,3,4-thiadiazin-3-ylmethyl)phenyl]carbamate;

3-pyrrolidinopropyl N-[4-(3,6-dihydro-5-(3-cyclopentyloxy-4-methoxyphenyl)-2-oxo-2H-1,3,4-thiadiazin-3-ylmethyl)phenyl]carbamate;

3-(N,N-diethylamino)propyl N-[4-(3,6-dihydro-5-(3-propoxy-4-methoxyphenyl)-2-oxo-2H-1,3,4-thiadiazin-3-ylmethyl)phenyl]carbamate;

2-(N,N-diethylamino)ethyl N-[4-(3,6-dihydro-5-(3-propoxy-4-methoxyphenyl)-2-oxo-2H-1,3,4-thiadiazin-3-ylmethyl)phenyl]carbamate;

2-(N,N-diisopropylamino)ethyl N-[4-(3,6-dihydro-5-(3-propoxy-4-methoxyphenyl)-2-oxo-2H-1,3,4-thiadiazin-3-ylmethyl)phenyl]carbamate;

6-(N,N-dimethylamino)hexyl N-[4-(3,6-dihydro-5-(3-propoxy-4-methoxyphenyl)-2-oxo-2H-1,3,4-thiadiazin-3-ylmethyl)phenyl]carbamate;

2-(N,N-dimethylamino)-1-methylethyl N-[4-(3,6-dihydro-5-(3-propoxy-4-methoxyphenyl)-2-oxo-2H-1,3,4-thiadiazin-3-ylmethyl)phenyl]carbamate;

1-methyl-4-piperidyl N-[4-(3,6-dihydro-5-(3-propoxy-4-methoxyphenyl)-2-oxo-2H-1,3,4-thiadiazin-3-ylmethyl)phenyl]carbamate;

2-morpholinoethyl N-[4-(3,6-dihydro-5-(3-propoxy-4-methoxyphenyl)-2-oxo-2H-1,3,4-thiadiazin-3-ylmethyl)phenyl]carbamate;

3-morpholinopropyl N-[4-(3,6-dihydro-5-(3-propoxy-4-methoxyphenyl)-2-oxo-2H-1,3,4-thiadiazin-3-ylmethyl)phenyl]carbamate;

2-thiomorpholinoethyl N-[4-(3,6-dihydro-5-(3-propoxy-4-methoxyphenyl)-2-oxo-2H-1,3,4-thiadiazin-3-ylmethyl)phenyl]carbamate;

3-thiomorpholinopropyl N-[4-(3,6-dihydro-5-(3-propoxy-4-methoxyphenyl)-2-oxo-2H-1,3,4-thiadiazin-3-ylmethyl)phenyl]carbamate;

2-piperazinoethyl N-[4-(3,6-dihydro-5-(3-propoxy-4-methoxyphenyl)-2-oxo-2H-1,3,4-thiadiazin-3-ylmethyl)phenyl]carbamate;

3-piperazinopropyl N-[4-(3,6-dihydro-5-(3-propoxy-4-methoxyphenyl)-2-oxo-2H-1,3,4-thiadiazin-3-ylmethyl)phenyl]carbamate;

2-pyrrolidinoethyl N-[4-(3,6-dihydro-5-(3-propoxy-4-methoxyphenyl)-2-oxo-2H-1,3,4-thiadiazin-3-ylmethyl)phenyl]carbamate;

3-pyrrolidinopropyl N-[4-(3,6-dihydro-5-(3-propoxy-4-methoxyphenyl)-2-oxo-2H-1,3,4-thiadiazin-3-ylmethyl)phenyl]carbamate;

3-(N,N-diethylamino)propyl N-[4-(3,-dihydro-5-(3-isopropoxy-4-methoxyphenyl)-2-oxo-2H-1,3,4-thiadiazin-3-ylmethyl)phenyl]carbamate;

2-(N,N-diethylamino)ethyl N-[4-(3,6-dihydro-5-(3-isopropoxy-4-methoxyphenyl)-2-oxo-2H-1,3,4-thiadiazin-3-ylmethyl)phenyl]carbamate;

2-(N,N-diisopropylamino)ethyl N-[4-(3,6-dihydro-5-(3-isopropoxy-4-methoxyphenyl)-2-oxo-2H-1,3,4-thiadiazin-3-ylmethyl)phenyl]carbamate;

6-(N,N-dimethylamino)hexyl N-[4-(3,6-dihydro-5-(3-isopropoxy-4-methoxyphenyl)-2-oxo-2H-1,3,4-thiadiazin-3-ylmethyl)phenyl]carbamate;

2-(N,N-dimethylamino)-1-methylethyl N-[4-(3,6-dihydro-5-(3-isopropoxy-4-methoxyphenyl)-2-oxo-2H-1,3,4-thiadiazin-3-ylmethyl)phenyl]carbamate;

1-methyl-4-piperidyl N-[4-(3,6-dihydro-5-(3-isopropoxy-4-methoxyphenyl)-2-oxo-2H-1,3,4-thiadiazin-3-ylmethyl)phenyl]carbamate;

2-morpholinoethyl N-[4-(3,6-dihydro-5-(3-isopropoxy-4-methoxyphenyl)-2-oxo-2H-1,3,4-thiadiazin-3-ylmethyl)phenyl]carbamate;

3-morpholinopropyl N-[4-(3,6-dihydro-5-(3-isopropoxy-4-methoxyphenyl)-2-oxo-2H-1,3,4-thiadiazin-3-ylmethyl)phenyl]carbamate;

2-thiomorpholinoethyl N-[4-(3,6-dihydro-5-(3-isopropoxy-4-methoxyphenyl)-2-oxo-2H-1,3,4-thiadiazin-3-ylmethyl)phenyl]carbamate;

3-thiomorpholinopropyl N-[4-(3,6-dihydro-5-(3-isopropoxy-4-methoxyphenyl)-2-oxo-2H-1,3,4-thiadiazin-3-ylmethyl)phenyl]carbamate;

2-piperazinoethyl N-[4-(3,6-dihydro-5-(3-isopropoxy-4-methoxyphenyl)-2-oxo-2H-1,3,4-thiadiazin-3-ylmethyl)phenyl]carbamate;

3-piperazinopropyl N-[4-(3,6-dihydro-5-(3-isopropoxy-4-methoxyphenyl)-2-oxo-2H-1,3,4-thiadiazin-3-ylmethyl)phenyl]carbamate;

2-pyrrolidinoethyl N-[4-(3,6-dihydro-5-(3-isopropoxy-4-methoxyphenyl)-2-oxo-2H-1,3,4-thiadiazin-3-ylmethyl)phenyl]carbamate;

3-pyrrolidinopropyl N-[4-(3,6-dihydro-5-(3-isopropoxy-4-methoxyphenyl)-2-oxo-2H-1,3,4-thiadiazin-3-ylmethyl)phenyl]carbamate;

3-(N,N-diethylamino)propyl N-[4-(3,6-dihydro-5-(3-fluoromethoxy-4-methoxyphenyl)-2-oxo-2H-1,3,4-thiadiazin-3-ylmethyl)phenyl]carbamate;

2-(N,N-diethylamino)ethyl N-[4-(3,6-dihydro-5-(3-fluoromethoxy-4-methoxyphenyl)-2-oxo-2H-1,3,4-thiadiazin-3-ylmethyl)phenyl]carbamate;

2-(N,N-diisopropylamino)ethyl N-[4-(3,6-dihydro-5-(3-fluoromethoxy-4-methoxyphenyl)-2-oxo-2H-1,3,4-thiadiazin-3-ylmethyl)phenyl]carbamate;

6-(N,N-dimethylamino)hexyl N-[4-(3,6-dihydro-5-(3-fluoromethoxy-4-methoxyphenyl)-2-oxo-2H-1,3,4-thiadiazin-3-ylmethyl)phenyl]carbamate;

2-(N,N-dimethylamino)-1-methylethyl N-[4-(3,6-dihydro-5-(3-fluoromethoxy-4-methoxyphenyl)-2-oxo-2H-1,3,4-thiadiazin-3-ylmethyl)phenyl]carbamate;

1-methyl-4-piperidyl N-[4-(3,6-dihydro-5-(3-fluoromethoxy-4-methoxyphenyl)-2-oxo-2H-1,3,4-thiadiazin-3-ylmethyl)phenyl]carbamate;

2-morpholinoethyl N-[4-(3,6-dihydro-5-(3-fluoromethoxy-4-methoxyphenyl)-2-oxo-2H-1,3,4-thiadiazin-3-ylmethyl)phenyl]carbamate;

3-morpholinopropyl N-[4-(3,6-dihydro-5-(3-fluoromethoxy-4-methoxyphenyl)-2-oxo-2H-1,3,4-thiadiazin-3-ylmethyl)phenyl]carbamate;

2-thiomorpholinoethyl N-[4-(3,6-dihydro-5-(3-fluoromethoxy-4-methoxyphenyl)-2-oxo-2H-1,3,4-thiadiazin-3-ylmethyl)phenyl]carbamate;

3-thiomorpholinopropyl N-[4-(3,6-dihydro-5-(3-fluoromethoxy-4-methoxyphenyl)-2-oxo-2H-1,3,4-thiadiazin-3-ylmethyl)phenyl]carbamate;

2-piperazinoethyl N-[4-(3,6-dihydro-5-(3-fluoromethoxy-4-methoxyphenyl)-2-oxo-2H-1,3,4-thiadiazin-3-ylmethyl)phenyl]carbamate;

3-piperazinopropyl N-[4-(3,6-dihydro-5-(3-fluoromethoxy-4-methoxyphenyl)-2-oxo-2H-1,3,4-thiadiazin-3-ylmethyl)phenyl]carbamate;

2-pyrrolidinoethyl N-[4-(3,6-dihydro-5-(3-fluoromethoxy-4-methoxyphenyl)-2-oxo-2H-1,3,4-thiadiazin-3-ylmethyl)phenyl]carbamate;

3-pyrrolidinopropyl N-[4-(3,6-dihydro-5-(3-fluoromethoxy-4-methoxyphenyl)-2-oxo-2H-1,3,4-thiadiazin-3-ylmethyl)phenyl]carbamate;

N-[4-(3,6-dihydro-5-(3,4-dimethoxyphenyl)-2-oxo-2H-1,3,4-thiadiazin-3-ylmethyl)phenyl]-2-morpholinoacetamide;

N-[4-(3,6-dihydro-5-(3,4-dimethoxyphenyl)-2-oxo-2H-1,3,4-thiadiazin-3-ylmethyl)phenyl]-2-(N,N-diethylamino)acetamide;

N-[4-(3,6-dihydro-5-(3,4-dimethoxyphenyl)-2-oxo-2H-1,3,4-thiadiazin-3-ylmethyl)phenyl]-2-(N,N-diisopropylamino)acetamide;

N-[4-(3,6-dihydro-5-(3,4-dimethoxyphenyl)-2-oxo-2H-1,3,4-thiadiazin-3-ylmethyl)phenyl]-2-thiomorpholinoacetamide;

N-[4-(3,6-dihydro-5-(3,4-dimethoxyphenyl)-2-oxo-2H-1,3,4-thiadiazin-3-ylmethyl)phenyl]-2-piperidinoacetamide;

N-[4-(3,6-dihydro-5-(3,4-dimethoxyphenyl)-2-oxo-2H-1,3,4-thiadiazin-3-ylmethyl)phenyl]-2-(4-hydroxypiperidino)acetamide;

N-[4-(3,6-dihydro-5-(3,4-dimethoxyphenyl)-2-oxo-2H-1,3,4-thiadiazin-3-ylmethyl)phenyl]-2-piperazinoacetamide;

N-[4-(3,6-dihydro-5-(3,4-dimethoxyphenyl)-2-oxo-2H-1,3,4-thiadiazin-3-ylmethyl)phenyl]-2-(4-methylpiperazino)acetamide;

N-[4-(3,6-dihydro-5-(3,4-dimethoxyphenyl)-2-oxo-2H-1,3,4-thiadiazin-3-ylmethyl)phenyl]-2-pyrrolidinoacetamide;

N-[3-(3,6-dihydro-5-(3,4-dimethoxyphenyl)-2-oxo-2H-1,3,4-thiadiazin-3-ylmethyl)phenyl]-2-(4-morpholinyl)acetamide;

N-[3-(3,6-dihydro-5-(3,4-dimethoxyphenyl)-2-oxo-2H-1,3,4-thiadiazin-3-ylmethyl)phenyl]-2-(N,N-diethylamino)acetamide;

N-[3-(3,6-dihydro-5-(3,4-dimethoxyphenyl)-2-oxo-2H-1,3,4-thiadiazin-3-ylmethyl)phenyl]-2-(N,N-diisopropylamino)acetamide;

N-[3-(3,6-dihydro-5-(3,4-dimethoxyphenyl)-2-oxo-2H-1,3,4-thiadiazin-3-ylmethyl)phenyl]-2-thiomorpholinoacetamide;

N-[3-(3,6-dihydro-5-(3,4-dimethoxyphenyl)-2-oxo-2H-1,3,4-thiadiazin-3-ylmethyl)phenyl]-2-piperidinoacetamide;

N-[3-(3,6-dihydro-5-(3,4-dimethoxyphenyl)-2-oxo-2H-1,3,4-thiadiazin-3-ylmethyl)phenyl]-2-(4-hydroxypiperidino)acetamide;

N-[3-(3,6-dihydro-5-(3,4-dimethoxyphenyl)-2-oxo-2H-1,3,4-thiadiazin-3-ylmethyl)phenyl]-2-piperazinoacetamide;

N-[3-(3,6-dihydro-5-(3,4-dimethoxyphenyl)-2-oxo-2H-1,3,4-thiadiazin-3-ylmethyl)phenyl]-2-(4-methylpiperazino)acetamide;

N-[3-(3,6-dihydro-5-(3,4-dimethoxyphenyl)-2-oxo-2H-1,3,4-thiadiazin-3-ylmethyl)phenyl]-2-pyrrolidinoacetamide;

N-[4-(3,6-dihydro-5-(3,4-dimethoxyphenyl)-2-oxo-2H-1,3,4-thiadiazin-3-ylmethyl)phenyl]-3-(4-morpholinyl)propionamide;

N-[4-(3,6-dihydro-5-(3,4-dimethoxyphenyl)-2-oxo-2H-1,3,4-thiadiazin-3-ylmethyl)phenyl]-3-(N,N-diethylamino)propionamide;

N-[4-(3,6-dihydro-5-(3,4-dimethoxyphenyl)-2-oxo-2H-1,3,4-thiadiazin-3-ylmethyl)phenyl]-3-(N,N-diisopropylamino)propionamide;

N-[4-(3,6-dihydro-5-(3,4-dimethoxyphenyl)-2-oxo-2H-1,3,4-thiadiazin-3-ylmethyl)phenyl]-3-thiomorpholinopropionamide;

N-[4-(3,6-dihydro-5-(3,4-dimethoxyphenyl)-2-oxo-2H-1,3,4-thiadiazin-3-ylmethyl)phenyl]-3-piperidinopropionamide;

N-[4-(3,6-dihydro-5-(3,4-dimethoxyphenyl)-2-oxo-2H-1,3,4-thiadiazin-3-ylmethyl)phenyl]-3-(4-hydroxypiperidino)propionamide;

N-[4-(3,6-dihydro-5-(3,4-dimethoxyphenyl)-2-oxo-2H-1,3,4-thiadiazin-3-ylmethyl)phenyl]-3-piperazinopropionamide;

N-[4-(3,6-dihydro-5-(3,4-dimethoxyphenyl)-2-oxo-2H-1,3,4-thiadiazin-3-ylmethyl)phenyl]-3-pyrrolidinopropionamide;

N-[4-(3,6-dihydro-5-(3-ethoxy-4-methoxyphenyl)-2-oxo-2H-1,3,4-thiadiazin-3-ylmethyl)phenyl]-2-(4-morpholinyl)acetamide;

N-[4-(3,6-dihydro-5-(3-ethoxy-4-methoxyphenyl)-2-oxo-2H-1,3,4-thiadiazin-3-ylmethyl)phenyl]-2-(N,N-diethylamino)acetamide;

N-[4-(3,6-dihydro-5-(3-ethoxy-4-methoxyphenyl)-2-oxo-2H-1,3,4-thiadiazin-3-ylmethyl)phenyl]-2-(N,N-diisopropylamino)acetamide;

N-[4-(3,6-dihydro-5-(3-ethoxy-4-methoxyphenyl)-2-oxo-2H-1,3,4-thiadiazin-3-ylmethyl)phenyl]-2-thiomorpholinoacetamide;

N-[4-(3,6-dihydro-5-(3-ethoxy-4-methoxyphenyl)-2-oxo-2H-1,3,4-thiadiazin-3-ylmethyl)phenyl]-2-piperidinoacetamide;

N-[4-(3,6-dihydro-5-(3-ethoxy-4-methoxyphenyl)-2-oxo-2H-1,3,4-thiadiazin-3-ylmethyl)phenyl]-2-(4-hydroxypiperidino)acetamide;

N-[4-(3,6-dihydro-5-(3-ethoxy-4-methoxyphenyl)-2-oxo-2H-1,3,4-thiadiazin-3-ylmethyl)phenyl]-2-piperazinoacetamide;

N-[4-(3,6-dihydro-5-(3-ethoxy-4-methoxyphenyl)-2-oxo-2H-1,3,4-thiadiazin-3-ylmethyl)phenyl]-2-pyrrolidinoacetamide;

N-[4-(3,6-dihydro-5-(3-propoxy-4-methoxyphenyl)-2-oxo-2H-1,3,4-thiadiazin-3-ylmethyl)phenyl]-2-(4-morpholinyl)acetamide;

N-[4-(3,6-dihydro-5-(3-propoxy-4-methoxyphenyl)-2-oxo-2H-1,3,4-thiadiazin-3-ylmethyl)phenyl]-2-(N,N-diethylamino)acetamide;

N-[4-(3,6-dihydro-5-(3-propoxy-4-methoxyphenyl)-2-oxo-2H-1,3,4-thiadiazin-3-ylmethyl)phenyl]-2-(N,N-diisopropylamino)acetamide;

N-[4-(3,6-dihydro-5-(3-propxoy-4-methoxyphenyl)-2-oxo-2H-1,3,4-thiadiazin-3-ylmethyl)phenyl]-2-thiomorpholinoacetamide;

N-[4-(3,6-dihydro-5-(3-propoxy-4-methoxyphenyl)-2-oxo-2H-1,3,4-thiadiazin-3-ylmethyl)phenyl]-2-piperidinoacetamide;

N-[4-(3,6-dihydro-5-(3-propoxy-4-methoxyphenyl)-2-oxo-2H-1,3,4-thiadiazin-3-ylmethyl)phenyl]-2-(4-hydroxypiperidino)acetamide;

N-[4-(3,6-dihydro-5-(3-propoxy-4-methoxyphenyl)-2-oxo-2H-1,3,4-thiadiazin-3-ylmethyl)phenyl]-2-piperazinoacetamide;

N-[4-(3,6-dihydro-5-(3-propoxy-4-methoxyphenyl)-2-oxo-2H-1,3,4-thiadiazin-3-ylmethyl)phenyl]-2-pyrrolidinoacetamide;

N-[4-(3,6-dihydro-5-(3-isopropoxy-4-methoxyphenyl)-2-oxo-2H-1,3,4-thiadiazin-3-ylmethyl)phenyl]-2-(4-morpholinyl)acetamide;

N-[4-(3,6-dihydro-5-(3-isopropoxy-4-methoxyphenyl)-2-oxo-2H-1,3,4-thiadiazin-3-ylmethyl)phenyl]-2-(N,N-diethylamino)acetamide;

N-[4-(3,6-dihydro-5-(3-isopropoxy-4-methoxyphenyl)-2-oxo-2H-1,3,4-thiadiazin-3-ylmethyl)phenyl]-2-(N,N-diisopropylamino)acetamide;

N-[4-(3,6-dihydro-5-(3-isopropoxy-4-methoxyphenyl)-2-oxo-2H-1,3,4-thiadiazin-3-ylmethyl)phenyl]-2-thiomorpholinoacetamide;

N-[4-(3,6-dihydro-5-(3-isopropoxy-4-methoxyphenyl)-2-oxo-2H-1,3,4-thiadiazin-3-ylmethyl)phenyl]-2-piperidinoacetamide;

N-[4-(3,6-dihydro-5-(3-isopropoxy-4-methoxyphenyl)-2-oxo-2H-1,3,4-thiadiazin-3-ylmethyl)phenyl]-2-(4-hydroxypiperidino)acetamide;

N-[4-(3,6-dihydro-5-(3-isopropoxy-4-methoxyphenyl)-2-oxo-2H-1,3,4-thiadiazin-3-ylmethyl)phenyl]-2-piperazinoacetamide;

N-[4-(3,6-dihydro-5-(3-isopropoxy-4-methoxyphenyl)-2-oxo-2H-1,3,4-thiadiazin-3-ylmethyl)phenyl]-2-pyrrolidinoacetamide;

N-[4-(3,6-dihydro-5-(3-cyclopentyloxy-4-methoxyphenyl)-2-oxo-2H-1,3,4-thiadiazin-3-ylmethyl)phenyl]-2-(4-morpholinyl)acetamide;

N-[4-(3,6-dihydro-5-(3-cyclopentyloxy-4-methoxyphenyl)-2-oxo-2H-1,3,4-thiadiazin-3-ylmethyl)phenyl]-2-(N,N-diethylamino)acetamide;

N-[4-(3,6-dihydro-5-(3-cyclopentyloxy-4-methoxyphenyl)-2-oxo-2H-1,3,4-thiadiazin-3-ylmethyl)phenyl]-2-(N,N-diisopropylamino)acetamide;

N-[4-(3,6-dihydro-5-(3-cyclopentyloxy-4-methoxyphenyl)-2-oxo-2H-1,3,4-thiadiazin-3-ylmethyl)phenyl]-2-thiomorpholinoacetamide;

N-[4-(3,6-dihydro-5-(3-cyclopentyloxy-4-methoxyphenyl)-2-oxo-2H-1,3,4-thiadiazin-3-ylmethyl)phenyl]-2-piperidinoacetamide;

N-[4-(3,6-dihydro-5-(3-cyclopentyloxy-4-methoxyphenyl)-2-oxo-2H-1,3,4-thiadiazin-3-ylmethyl)phenyl]-2-(4-hydroxypiperidino)acetamide;

N-[4-(3,6-dihydro-5-(3-cyclopentyloxy-4-methoxyphenyl)-2-oxo-2H-1,3,4-thiadiazin-3-ylmethyl)phenyl]-2-pyrrolidinoacetamide;

N-[4-(3,6-dihydro-5-(3-ethoxy-4-methoxyphenyl)-2-oxo-2H-1,3,4-thiadiazin-3-ylmethyl)phenyl]-3-(4-morpholinyl)propionamide;

N-[4-(3,6-dihydro-5-(3-ethoxy-4-methoxyphenyl)-2-oxo-2H-1,3,4-thiadiazin-3-ylmethyl)phenyl]-3-(N,N-diethylamino)propionamide;

N-[4-(3,6-dihydro-5-(3-ethoxy-4-methoxyphenyl)-2-oxo-2H-1,3,4-thiadiazin-3-ylmethyl)phenyl]-3-(N,N-diisopropylamino)propionamide;

N-[4-(3,6-dihydro-5-(3-ethoxy-4-methoxyphenyl)-2-oxo-2H-1,3,4-thiadiazin-3-ylmethyl)phenyl]-3-thiomorpholinopropionamide;

N-[4-(3,6-dihydro-5-(3-ethoxy-4-methoxyphenyl)-2-oxo-2H-1,3,4-thiadiazin-3-ylmethyl)phenyl]-3-piperidinopropionamide;

N-[4-(3,6-dihydro-5-(3-ethoxy-4-methoxyphenyl)-2-oxo-2H-1,3,4-thiadiazin-3-ylmethyl)phenyl]-3-(4-hydroxypiperidino)propionamide;

N-[4-(3,6-dihydro-5-(3-ethoxy-4-methoxyphenyl)-2-oxo-2H-1,3,4-thiadiazin-3-ylmethyl)phenyl]-3-piperazinopropionamide;

N-[4-(3,6-dihydro-5-(3-ethoxy-4-methoxyphenyl)-2-oxo-2H-1,3,4-thiadiazin-3-ylmethyl)phenyl]-3-pyrrolidinopropionamide;

N-[4-(3,6-dihydro-5-(3-propoxy-4-methoxyphenyl)-2-oxo-2H-1,3,4-thiadiazin-3-ylmethyl)phenyl]-3-(4-morpholinyl)propionamide;

N-[4-(3,6-dihydro-5-(3-propoxy-4-methoxyphenyl)-2-oxo-2H-1,3,4-thiadiazin-3-ylmethyl)phenyl]-3-(N,N-diethylamino)propionamide;

N-[4-(3,6-dihydro-5-(3-propoxy-4-methoxyphenyl)-2-oxo-2H-1,3,4-thiadiazin-3-ylmethyl)phenyl]-3-(N,N-diisopropylamino)propionamide;

N-[4-(3,6-dihydro-5-(3-propoxy-4-methoxyphenyl)-2-oxo-2H-1,3,4-thiadiazin-3-ylmethyl)phenyl]-3-thiomorpholinopropionamide;

N-[4-(3,6-dihydro-5-(3-propoxy-4-methoxyphenyl)-2-oxo-2H-1,3,4-thiadiazin-3-ylmethyl)phenyl]-3-piperidinopropionamide;

N-[4-(3,6-dihydro-5-(3-propoxy-4-methoxyphenyl)-2-oxo-2H-1,3,4-thiadiazin-3-ylmethyl)phenyl]-3-(4-hydroxypiperidino)propionamide;

N-[4-(3,6-dihydro-5-(3-propoxy-4-methoxyphenyl)-2-oxo-2H-1,3,4-thiadiazin-3-ylmethyl)phenyl]-3-piperazinopropionamide;

N-[4-(3,6-dihydro-5-(3-propoxy-4-methoxyphenyl)-2-oxo-2H-1,3,4-thiadiazin-3-ylmethyl)phenyl]-3-pyrrolidinopropionamide.

N-[4-(3,6-dihydro-5-(3-isopropoxy-4-methoxyphenyl)-2-oxo-2H-1,3,4-thiadiazin-3-ylmethyl)phenyl]-3-(4-morpholinyl)propionamide;

N-[4-(3,6-dihydro-5-(3-isopropoxy-4-methoxyphenyl)-2-oxo-2H-1,3,4-thiadiazin-3-ylmethyl)phenyl]-3-(N,N-diethylamino)propionamide;

N-[4-(3,6-dihydro-5-(3-isopropoxy-4-methoxyphenyl)-2-oxo-2H-1,3,4-thiadiazin-3-ylmethyl)phenyl]-3-(N,N-diisopropylamino)propionamide;

N-[4-(3,6-dihydro-5-(3-isopropoxy-4-methoxyphenyl)-2-oxo-2H-1,3,4-thiadiazin-3-ylmethyl)phenyl]-3-thiomorpholinopropionamide;

N-[4-(3,6-dihydro-5-(3-isopropoxy-4-methoxyphenyl)-2-oxo-2H-1,3,4-thiadiazin-3-ylmethyl)phenyl]-3-piperidinopropionamide;

N-[4-(3,6-dihydro-5-(3-isopropoxy-4-methoxyphenyl)-2-oxo-2H-1,3,4-thiadiazin-3-ylmethyl)phenyl]-3-(4-hydroxypiperidino)propionamide;

N-[4-(3,6-dihydro-5-(3-isopropoxy-4-methoxyphenyl)-2-oxo-2H-1,3,4-thiadiazin-3-ylmethyl)phenyl]-3-piperazinopropionamide;

N-[4-(3,6-dihydro-5-(3-isopropoxy-4-methoxyphenyl)-2-oxo-2H-1,3,4-thiadiazin-3-ylmethyl)phenyl]-3-pyrrolidinopropionamide;

N-[4-(3,6-dihydro-5-(3-cyclopentyloxy-4-methoxyphenyl)-2-oxo-2H-1,3,4-thiadiazin-3-ylmethyl)phenyl]-3-(4-morpholinyl)propionamide;

N-[4-(3,6-dihydro-5-(3-cyclopentyloxy-4-methoxyphenyl)-2-oxo-2H-1,3,4-thiadiazin-3-ylmethyl)phenyl]-3-(N,N-diethylamino)propionamide;

N-[4-(3,6-dihydro-5-(3-cyclopentyloxy-4-methoxyphenyl)-2-oxo-2H-1,3,4-thiadiazin-3-ylmethyl)phenyl]-3-(N,N-diisopropylamino)propionamide;

N-[4-(3,6-dihydro-5-(3-cyclopentyloxy-4-methoxyphenyl)-2-oxo-2H-1,3,4-thiadiazin-3-ylmethyl)phenyl]-3-thiomorpholinopropionamide;

N-[4-(3,6-dihydro-5-(3-cyclopentyloxy-4-methoxyphenyl)-2-oxo-2H-1,3,4-thiadiazin-3-ylmethyl)phenyl]-3-piperidinopropionamide;

N-[4-(3,6-dihydro-5-(3-cyclopentyloxy-4-methoxyphenyl)-2-oxo-2H-1,3,4-thiadiazin-3-ylmethyl)phenyl]-3-(4-hydroxypiperidino)propionamide;

N-[4-(3,6-dihydro-5-(3-cyclopentyloxy-4-methoxyphenyl)-2-oxo-2H-1,3,4-thiadiazin-3-ylmethyl)phenyl]-3-piperazinopropionamide;

N-[4-(3,6-dihydro-5-(3-cyclopentyloxy-4-methoxyphenyl)-2-oxo-2H-1,3,4-thiadiazin-3-ylmethyl)phenyl]-3-pyrrolidinopropionamide;

3-(N,N-diethylamino)propyl N-[4-(6-(3,4-dimethoxyphenyl)-3-oxo-2,3,4,5-tetrahydropyridazin-2-ylmethyl)phenyl]carbamate;

3-(N,N-diethylamino)propyl N-[4-(6-(3,4-dimethoxyphenyl)-3-oxo-2,3,4,5-tetrahydropyridazin-2-ylmethyl)phenyl]carbamate;

3-(N,N-diisopropylamino)ethyl N-[4-(6-(3,4-dimethoxyphenyl)-3-oxo-2,3,4,5-tetrahydropyridazin-2-ylmethyl)phenyl]carbamate;

6-(N,N-dimethylamino)hexyl N-[4-(6-(3,4-dimethoxyphenyl)-3-oxo-2,3,4,5-tetrahydropyridazin-2-ylmethyl)phenyl]carbamate;

2-(N,N-dimethylamino)-1-methylethyl N-[4-(6-(3,4-dimethoxyphenyl)-3-oxo-2,3,4,5-tetrahydropyridazin-2-ylmethyl)phenyl]carbamate;

1-methyl-4-piperidyl N-[4-(6-(3,4-dimethoxyphenyl)-3-oxo-2,3,4,5-tetrahydropyridazin-2-ylmethyl)phenyl]carbamate;

(4-hydroxy-1-piperidyl)ethyl N-[4-(6-(3,4-dimethoxyphenyl)-3-oxo-2,3,4,5-tetrahydropyridazin-2-ylmethyl)phenyl]carbamate;

2-morpholinoethyl N-[4-(6-(3,4-dimethoxyphenyl)-3-oxo-2,3,4,5-tetrahydropyridazin-2-ylmethyl)phenyl]carbamate;

3-morpholinopropyl N-[4-(6-(3,4-dimethoxyphenyl)-3-oxo-2,3,4,5-tetrahydropyridazin-2-ylmethyl)phenyl]carbamate;

2-thiomorpholinoethyl N-[4-(6-(3,4-dimethoxyphenyl)-3-oxo-2,3,4,5-tetrahydropyridazin-2-ylmethyl)phenyl]carbamate;

3-thiomorpholinopropyl N-[4-(6-(3,4-dimethoxyphenyl)-3-oxo-2,3,4,5-tetrahydropyridazin-2-ylmethyl)phenyl]carbamate;

2-piperazinoethyl N-[4-(6-(3,4-dimethoxyphenyl)-3-oxo-2,3,4,5-tetrahydropyridazin-2-ylmethyl)phenyl]carbamate;

3-piperazinopropyl N-[4-(6-(3,4-dimethoxyphenyl)-3-oxo-2,3,4,5-tetrahydropyridazin-2-ylmethyl)phenyl]carbamate;

2-pyrrolidinoethyl N-[4-(6-(3,4-dimethoxyphenyl)-3-oxo-2,3,4,5-tetrahydropyridazin-2-ylmethyl)phenyl]carbamate;

3-pyrrolidinopropyl N-[4-(6-(3,4-dimethoxyphenyl)-3-oxo-2,3,4,5-tetrahydropyridazin-2-ylmethyl)phenyl]carbamate;

3-(N,N-diethylamino)propyl N-[4-(6-(3-ethoxy-4-methoxyphenyl)-3-oxo-2,3,4,5-tetrahydropyridazin-2-ylmethyl)phenyl]carbamate;

2-(N,N-diethylamino)ethyl N-[4-(6-(3-ethoxy-4-methoxyphenyl)-3-oxo-2,3,4,5-tetrahydropyridazin-2-ylmethyl)phenyl]carbamate;

2-(N,N-diisopropylamino)ethyl N-[4-(6-(3-ethoxy-4-methoxyphenyl)-3-oxo-2,3,4,5-tetrahydropyridazin-2-ylmethyl)phenyl]carbamate;

6-(N,N-dimethylamino)hexyl N-[4-(6-(3-ethoxy-4-methoxyphenyl)-3-oxo-2,3,4,5-tetrahydropyridazin-2-ylmethyl)phenyl]carbamate;

2-(N,N-dimethylamino)-1-methylethyl N-[4-(6-(3-ethoxy-4-methoxyphenyl)-3-oxo-2,3,4,5-tetrahydropyridazin-2-ylmethyl)phenyl]carbamate;

1-methyl-4-piperidyl N-[4-(6-(3-ethoxy-4-methoxyphenyl)-3-oxo-2,3,4,5-tetrahydropyridazin-2-ylmethyl)phenyl]carbamate;

2-morpholinoethyl N-[4-(6-(3-ethoxy-4-methoxyphenyl)-3-oxo-2,3,4,5-tetrahydropyridazin-2-ylmethyl)phenyl]carbamate;

3-morpholinopropyl N-[4-(6-(3-ethoxy-4-methoxyphenyl)-3-oxo-2,3,4,5-tetrahydropyridazin-2-ylmethyl)phenyl]carbamate;

2-thiomorpholinoethyl N-[4-(6-(3-ethoxy-4-methoxyphenyl)-3-oxo-2,3,4,5-tetrahydropyridazin-2-ylmethyl)phenyl]carbamate;

3-thiomorpholinopropyl N-[4-(6-(3-ethoxy-4-methoxyphenyl)-3-oxo-2,3,4,5-tetrahydropyridazin-2-ylmethyl)phenyl]carbamate;

2-piperazinoethyl N-[4-(6-(3-ethoxy-4-methoxyphenyl)-3-oxo-2,3,4,5-tetrahydropyridazin-2-ylmethyl)phenyl]carbamate;

3-piperazinopropyl N-[4-(6-(3-ethoxy-4-methoxyphenyl)-3-oxo-2,3,4,5-tetrahydropyridazin-2-ylmethyl)phenyl]carbamate;

2-pyrrolidinoethyl N-[4-(6-(3-ethoxy-4-methoxyphenyl)-3-oxo-2,3,4,5-tetrahydropyridazin-2-ylmethyl)phenyl]carbamate;

3-pyrrolidinopropyl N-[4-(6-(3-ethoxy-4-methoxyphenyl)-3-oxo-2,3,4,5-tetrahydropyridazin-2-ylmethyl)phenyl]carbamate.

3-(N,N-diethylamino)propyl N-[4-(6-(3,4-dimethoxyphenyl)-3-oxo-5-ethyl-2,3,4,5-tetrahydropyridazin-2-ylmethyl)phenyl]carbamate;

2-(N,N-diethylamino)ethyl N-[4-(6-(3,4-dimethoxyphenyl)-3-oxo-5-ethyl-2,3,4,5-tetrahydropyridazin-2-ylmethyl)phenyl]carbamate;

2-(N,N-diisopropylamino)ethyl N-[4-(6-(3,4-dimethoxyphenyl)-3-oxo-5-ethyl-2,3,4,5-tetrahydropyridazin-2-ylmethyl)phenyl]carbamate;

6-(N,N-dimethylamino)hexyl N-[4-(6-(3,4-dimethoxyphenyl)-3-oxo-5-ethyl-2,3,4,5-tetrahydropyridazin-2-ylmethyl)phenyl]carbamate;

2-(N,N-dimethylamino)-1-methylethyl N-[4-(6-(3,4-dimethoxyphenyl)-3-oxo-5-ethyl-2,3,4,5-tetrahydropyridazin-2-ylmethyl)phenyl]carbamate;

1-methyl-4-piperidylN-[4-(5-(3,4-dimethoxyphenyl)-3-oxo-5-ethyl-2,3,4,5-tetrahydropyridazin-2-ylmethyl)phenyl]carbamate;

2-morpholinoethyl N-[4-(6-(3,4-dimethoxyphenyl)-3-oxo-5-ethyl-2,3,4,5-tetrahydropyridazin-2-ylmethyl)phenyl]carbamate;

3-morpholinopropylN-[4-(6-(3,4-dimethoxyphenyl)-3-oxo-5-ethyl-2,3,4,5-tetrahydropyridazin-2-ylmethyl)phenyl]carbamate;

2-thiomorpholinoethyl N-[4-(6-(3,4-dimethoxyphenyl)-3-oxo-5-ethyl-2,3,4,5-tetrahydropyridazin-2-ylmethyl)phenyl]carbamate;

3-thiomorpholinopropyl N-[4-(6-(3,4-dimethoxyphenyl)-3-oxo-5-ethyl-2,3,4,5-tetrahydropyridazin-2-ylmethyl)phenyl]carbamate;

2-piperazinoethyl N-[4-(6-(3,4-dimethoxyphenyl)-3-oxo-5-ethyl-2,3,4,5-tetrahydropyridazin-2-ylmethyl)phenyl]carbamate;

3-piperazinopropyl N-[4-(6-(3,4-dimethoxyphenyl)-3-oxo-5-ethyl-2,3,4,5-tetrahydropyridazin-2-ylmethyl)phenyl]carbamate;

2-pyrrolidinoethyl N-[4-(6-(3,4-dimethoxyphenyl)-3-oxo-5-ethyl-2,3,4,5-tetrahydropyridazin-2-ylmethyl)phenyl]carbamate;

3-pyrrolidinopropyl N-[4-(6-(3,4-dimethoxyphenyl)-3-oxo-5-ethyl-2,3,4,5-tetrahydropyridazin-2-ylmethyl)phenyl]carbamate;

3-(N,N-diethylamino)propyl N-[4-(6-(3-ethoxy-4-methoxyphenyl)-3-oxo-5-ethyl-2,3,4,5-tetrahydropyridazin-2-ylmethyl)phenyl]carbamate;

2-(N,N-diethylamino)ethyl N-[4-(6-(3-ethoxy-4-methoxyphenyl)-3-oxo-5-ethyl-2,3,4,5-tetrahydropyridazin-2-ylmethyl)phenyl]carbamate;

2-(N,N-diisopropylamino)ethyl N-[4-(6-(3-ethoxy-4-methoxyphenyl)-3-oxo-5-ethyl-2,3,4,5-tetrahydropyridazin-2-ylmethyl)phenyl]carbamate;

6-(N,N-dimethylamino)hexyl N-[4-(6-(3-ethoxy-4-methoxyphenyl)-3-oxo-5-ethyl-2,3,4,5-tetrahydropyridazin-2-ylmethyl)phenyl]carbamate;

2-(N,N-dimethylamino)-1-methylethyl N-[4-(6-(3-ethoxy-4-methoxyphenyl)-3-oxo-5-ethyl-2,3,4,5-tetrahydropyridazin-2-ylmethyl)phenyl]carbamate;

1-methyl-4-piperidyl N-[4-(6-(3-ethoxy-4-methoxyphenyl)-3-oxo-5-ethyl-2,3,4,5-tetrahydropyridazin-2-ylmethyl)phenyl]carbamate;

2-morpholinoethyl N-[4-(6-(3-ethoxy-4-methoxyphenyl)-3-oxo-5-ethyl-2,3,4,5-tetrahydropyridazin-2-ylmethyl)phenyl]carbamate;

3-morpholinopropyl N-[4-(6-(3-ethoxy-4-methoxyphenyl)-3-oxo-5-ethyl-2,3,4,5-tetrahydropyridazin-2-ylmethyl)phenyl]carbamate;

2-thiomorpholinoethylN-[4-(6-(3-ethoxy-4-methoxyphenyl)-3-oxo-5-ethyl-2,3,4,5-tetrahydropyridazin-2-ylmethyl)phenyl]carbamate;

3-thiomorpholinopropylN-[4-(6-(3-ethoxy-4-methoxyphenyl)-3-oxo-5-ethyl-2,3,4,5-tetrahydropyridazin-2-ylmethyl)phenyl]carbamate;

2-piperazinoethyl N-[4-(6-(3-ethoxy-4-methoxyphenyl)-3-oxo-5-ethyl-2,3,4,5-tetrahydropyridazin-2-ylmethyl)phenyl]carbamate;

3-piperazinopropyl N-[4-(6-(3-ethoxy-4-methoxyphenyl)-3-oxo-5-ethyl-2,3,4,5-tetrahydropyridazin-2-ylmethyl)phenyl]carbamate;

2-pyrrolidinoethyl N-[4-(6-(3-ethoxy-4-methoxyphenyl)-3-oxo-5-ethyl-2,3,4,5-tetrahydropyridazin-2-ylmethyl)phenyl]carbamate;

3-pyrrolidinopropyl N-[4-(6-(3-ethoxy-4-methoxyphenyl)-3-oxo-5-ethyl-2,3,4,5-tetrahydropyridazin-2-ylmethyl)phenyl]carbamate;

3-(N,N-diethylamino)propyl N-[4-(6-(3-cyclopentyloxy-4-methoxyphenyl)-3-oxo-2,3,4,5-tetrahydropyridazin-2-ylmethyl)phenyl]carbamate;

2-(N,N-diethylamino)ethyl N-[4-(6-(3-cyclopentyloxy-4-methoxyphenyl)-3-oxo-2,3,4,5-tetrahydropyridazin-2-ylmethyl)phenyl]carbamate;

2-(N,N-diisopropylamino)ethyl N-[4-(6-(3-cyclopentyloxy-4-methoxyphenyl)-3-oxo-2,3,4,5-tetrahydropyridazin-2-ylmethyl)phenyl]carbamate;

6-(N,N-dimethylamino)hexyl N-[4-(6-(3-cyclopentyloxy-4-methoxyphenyl)-3-oxo-2,3,4,5-tetrahydropyridazin-2-ylmethyl)phenyl]carbamate;

2-(N,N-dimethylamino)-1-methylethyl N-[4-(6-(3-cyclopentyloxy-4-methoxyphenyl)-3-oxo-2,3,4,5-tetrahydropyridazin-2-ylmethyl)phenyl]carbamate;

1-methyl-4-piperidylN-[4-(6-(3-cyclopentyloxy-4-methoxyphenyl)-3-oxo-2,3,4,5-tetrahydropyridazin-2-ylmethyl)phenyl]carbamate;

2-morpholinoethyl N-[4-(6-(3-cyclopentyloxy-4-methoxyphenyl)-3-oxo-2,3,4,5-tetrahydropyridazin-2-ylmethyl)phenyl]carbamate;

3-morpholinopropyl N-[4-(6-(3-cyclopentyloxy-4-methoxyphenyl)-3-oxo-2,3,4,5-tetrahydropyridazin-2-ylmethyl)phenyl]carbamate;

2-thiomorpholinoethyl N-[4-(6-(3-cyclopentyloxy-4-methoxyphenyl)-3-oxo-2,3,4,5-tetrahydropyridazin-2-ylmethyl)phenyl]carbamate;

3-thiomorpholinopropyl N-[4-(6-(3-cyclopentyloxy-4-methoxyphenyl)-3-oxo-2,3,4,5-tetrahydropyridazin-2-ylmethyl)phenyl]carbamate;

2-piperazinoethyl N-[4-(6-(3-cyclopentyloxy-4-methoxyphenyl)-3-oxo-2,3,4,5-tetrahydropyridazin-2-ylmethyl)phenyl]carbamate;

3-piperazinopropyl N-[4-(6-(3-cyclopentyloxy-4-methoxyphenyl)-3-oxo-2,3,4,5-tetrahydropyridazin-2-ylmethyl)phenyl]carbamate;

2-pyrrolidinoethyl N-[4-(6-(3-cyclopentyloxy-4-methoxyphenyl)-3-oxo-2,3,4,5-tetrahydropyridazin-2-ylmethyl)phenyl]carbamate;

3-pyrrolidinopropyl N-[4-(6-(3-cyclopentyloxy-4-methoxyphenyl)-3-oxo-2,3,4,5-tetrahydropyridazin-2-ylmethyl)phenyl]carbamate;

3-(N,N-diethylamino)propyl N-[4-(6-(3-propoxy-4-methoxyphenyl)-3-oxo-2,3,4,5-tetrahydropyridazin-2-ylmethyl)phenyl]carbamate;

2-(N,N-diethylamino)ethyl N-[4-(6-(3-propoxy-4-methoxyphenyl)-3-oxo-2,3,4,5-tetrahydropyridazin-2-ylmethyl)phenyl]carbamate;

2-(N,N-diisopropylamino)ethyl N-[4-(6-(3-propoxy-4-methoxyphenyl)-3-oxo-2,3,4,5-tetrahydropyridazin-2-ylmethyl)phenyl]carbamate;

6-(N,N-dimethylamino)hexyl N-[4-(6-(3-propoxy-4-methoxyphenyl)-3-oxo-2,3,4,5-tetrahydropyridazin-2-ylmethyl)phenyl]carbamate;

2-(N,N-dimethylamino)-1-methylethyl N-[4-(6-(3-propoxy-4-methoxyphenyl)-3-oxo-2,3,4,5-tetrahydropyridazin-2-ylmethyl)phenyl]carbamate;

1-methyl-4-piperidyl N-[4-(6-(3-propoxy-4-methoxyphenyl)-3-oxo-2,3,4,5-tetrahydropyridazin-2-ylmethyl)phenyl]carbamate;

2-morpholinoethyl N-[4-(6-(3-propoxy-4-methoxyphenyl)-3-oxo-2,3,4,5-tetrahydropyridazin-2-ylmethyl)phenyl]carbamate;

3-morpholinopropyl N-[4-(6-(3-propoxy-4-methoxyphenyl)-3-oxo-2,3,4,5-tetrahydropyridazin-2-ylmethyl)phenyl]carbamate;

2-thiomorpholinoethyl N-[4-(6-(3-propoxy-4-methoxyphenyl)-3-oxo-2,3,4,5-tetrahydropyridazin-2-ylmethyl)phenyl]carbamate;

3-thiomorpholinopropyl N-[4-(6-(3-propoxy-4-methoxyphenyl)-3-oxo-2,3,4,5-tetrahydropyridazin-2-ylmethyl)phenyl]carbamate;

2-piperazinoethyl N-[4-(6-(3-propoxy-4-methoxyphenyl)-3-oxo-2,3,4,5-tetrahydropyridazin-2-ylmethyl)phenyl]carbamate;

3-piperazinopropyl N-[4-(6-(3-propoxy-4-methoxyphenyl)-3-oxo-2,3,4,5-tetrahydropyridazin-2-ylmethyl)phenyl]carbamate;

2-pyrrolidinoethyl N-[4-(6-(3-propoxy-4-methoxyphenyl)-3-oxo-2,3,4,5-tetrahydropyridazin-2-ylmethyl)phenyl]carbamate;

3-pyrrolidinopropyl N-[4-(6-(3-propoxy-4-methoxyphenyl)-3-oxo-2,3,4,5-tetrahydropyridazin-2-ylmethyl)phenyl]carbamate;

3-(N,N-diethylamino)propyl N-[4-(6-(3-isopropoxy-4-methoxyphenyl)-3-oxo-2,3,4,5-tetrahydropyridazin-2-ylmethyl)phenyl]carbamate;

2-(N,N-diethylamino)ethyl N-[4-(6-(3-isopropoxy-4-methoxyphenyl)-3-oxo-2,3,4,5-tetrahydropyridazin-2-ylmethyl)phenyl]carbamate;

2-(N,N-diisopropylamino)ethyl N-[4-(6-(3-isopropoxy-4-methoxyphenyl)-3-oxo-2,3,4,5-tetrahydropyridazin-2-ylmethyl)phenyl]carbamate;

6-(N,N-dimethylamino)hexyl N-[4-(6-(3-isopropoxy-4-methoxyphenyl)-3-oxo-2,3,4,5-tetrahydropyridazin-2-ylmethyl)phenyl]carbamate;

2-(N,N-dimethylamino)-1-methylethyl N-[4-(6-(3-isopropoxy-4-methoxyphenyl)-3-oxo-2,3,4,5-tetrahydropyridazin-2-ylmethyl)phenyl]carbamate;

1-methyl-4-piperidyl N-[4-(6-(3-isopropoxy-4-methoxyphenyl)-3oxo-2,3,4,5-tetrahydropyridazin-2-ylmethyl)phenyl]carbamate;

2-morpholinoethyl N-[4-(6-(3-isopropoxy-4-methoxyphenyl)-3-oxo-2,3,4,5-tetrahydropyridazin-2-ylmethyl)phenyl]carbamate;

3-morpholinopropyl N-[4-(6-(3-isopropoxy-4-methoxyphenyl)-3-oxo-2,3,4,5-tetrahydropyridazin-2-ylmethyl)phenyl]carbamate;

2-thiomorpholinoethyl N-[4-(6-(3-isopropoxy-4-methoxyphenyl)-3-oxo-2,3,4,5-tetrahydropyridazin-2-ylmethyl)phenyl)carbamate;

3-thiomorpholinopropyl N-[4-(6-(3-isopropoxy-4-methoxyphenyl)-3-oxo-2,3,4,5-tetrahydropyridazin-2-ylmethyl)phenyl]carbamate;

2-piperazinoethyl N-[4-(6-(3-isopropoxy-4-methoxyphenyl)-3-oxo-2,3,4,5-tetrahydropyridazin-2-ylmethyl)phenyl]carbamate;

3-piperazinopropyl N-[4-(6-(3-isopropoxy-4-methoxyphenyl)-3-oxo-2,3,4,5-tetrahydropyridazin-2-ylmethyl)phenyl]carbamate;

2-pyrrolidinoethyl N-[4-(6-(3-isopropoxy-4-methoxyphenyl)-3-oxo-2,3,4,5-tetrahydropyridazin-2-ylmethyl)phenyl]carbamate;

3-pyrrolidinopropyl N-[4-(6-(3-isopropoxy-4-methoxyphenyl)-3-oxo-2,3,4,5-tetrahydropyridazin-2-ylmethyl)phenyl]carbamate;

3-(N,N-diethylamino)propyl N-[4-(6-(3-fluoromethoxy-4-methoxyphenyl)-3-oxo-2,3,4,5-tetrahydropyridazin-2-ylmethyl)phenyl]carbamate;

2-(N,N-diethylamino)ethyl N-[4-(6-(3-fluoromethoxy-4-methoxyphenyl)-3-oxo-2,3,4,5-tetrahydropyridazin-2-ylmethyl)phenyl]carbamate;

2-(N,N-diisopropylamino)ethyl N-[4-(6-(3-fluoromethoxy-4-methoxyphenyl)-3-oxo-2,3,4,5-tetrahydropyridazin-2-ylmethyl)phenyl]carbamate;

6-(N,N-dimethylamino)hexyl N-[4-(6-(3-fluoromethoxy-4-methoxyphenyl)-3-oxo-2,3,4,5-tetrahydropyridazin-2-ylmethyl)phenyl]carbamate;

2-(N,N-dimethylamino)-1-methylethyl N-[4-(6-(3-fluoromethoxy-4-methoxyphenyl)-3-oxo-2,3,4,5-tetrahydropyridazin-2-ylmethyl)phenyl]carbamate;

1-methyl-4-piperidyl N-[4-(6-(3-fluoromethoxy-4-methoxyphenyl)-3-oxo-2,3,4,5-tetrahydropyridazin-2-ylmethyl)phenyl]carbamate;

2-morpholinoethyl N-[4-(6-(3-fluoromethoxy-4-methoxyphenyl)-3-oxo-2,3,4,5-tetrahydropyridazin-2-ylmethyl)phenyl]carbamate;

3-morpholinopropyl N-[4-(6-(3-fluoromethoxy-4-methoxyphenyl)-3-oxo-2,3,4,5-tetrahydropyridazin-2-ylmethyl)phenyl]carbamate;

2-thiomorpholinoethyl N-[4-(6-(3-fluoromethoxy-4-methoxyphenyl)-3-oxo-2,3,4,5-tetrahydropyridazin-2-ylmethyl)phenyl]carbamate;

3-thiomorpholinopropyl N-[4-(6-(3-fluoromethoxy-4-methoxyphenyl)-3-oxo-2,3,4,5-tetrahydropyridazin-2-ylmethyl)phenyl]carbamate;

2-piperazinoethyl N-[4-(6-(3-fluoromethoxy-4-methoxyphenyl)-3-oxo-2,3,4,5-tetrahydropyridazin-2-ylmethyl)phenyl]carbamate;

3-piperazinopropyl N-[4-(6-(3-fluoromethoxy-4-methoxyphenyl)-3-oxo-2,3,4,5-tetrahydropyridazin-2-ylmethyl)phenyl]carbamate;

2-pyrrolidinoethyl N-[4-(6-(3-fluoromethoxy-4-methoxyphenyl)-3-oxo-2,3,4,5-tetrahydropyridazin-2-ylmethyl)phenyl]carbamate;

3-pyrrolidinopropyl N-(8 4-(6-(3-fluoromethoxy-4-methoxyphenyl)-3-oxo-2,3,4,5-tetrahydropyridazin-2-ylmethyl)phenyl]carbamate.

N-[4-(6-(3-ethoxy-4-methoxyphenyl)-3-oxo-2,3,4,5-tetrahydropyridazin-2-ylmethyl)phenyl]-2-(4-morpholinyl)acetamide, m.p. 150°;

N-[4-(6-(3-ethoxy-4-methoxyphenyl)-3-oxo-2,3,4,5-tetrahydropyridazin-2-ylmethyl)phenyl]-2-(N,N-diethylamino)acetamide;

N-[4-(6-(3-ethoxy-4-methoxyphenyl)-3-oxo-2,3,4,5-tetrahydropyridazin-2-ylmethyl)phenyl]-2-(N,N-diisopropylamino)acetamide;

N-[4-(6-(3-ethoxy-4-methoxyphenyl)-3-oxo-2,3,4,5-tetrahydropyridazin-2-ylmethyl)phenyl]-2-thiomorpholinoacetamide;

N-[4-(6-(3-ethoxy-4-methoxyphenyl)-3-oxo-2,3,4,5-tetrahydropyridazin-2-ylmethyl)phenyl]-2-piperidinoacetamide;

N-[4-(6-(3-ethoxy-4-methoxyphenyl)-3-oxo-2,3,4,5-tetrahydropyridazin-2-ylmethyl)phenyl]-2-(4-hydroxypiperidino)acetamide;

N-[4-(6-(3-ethoxy-4-methoxyphenyl)-3-oxo-2,3,4,5-tetrahydropyridazin-2-ylmethyl)phenyl]-2-piperazinoacetamide;

N-[4-(6-(3-ethoxy-4-methoxyphenyl)-3-oxo-2,3,4,5-tetrahydropyridazin-2-ylmethyl)phenyl]-2-pyrrolidinoacetamide;

N-[4-(6-(3,4-dimethoxyphenyl)-3-oxo-2,3,4,5-tetrahydropyridazin-2-ylmethyl)phenyl]-2-(4-morpholinyl)acetamide;

N-[4-(6-(3,4-dimethoxyphenyl)-3-oxo-2,3,4,5-tetrahydropyridazin-2-ylmethyl)phenyl]-2-(N,N-diethylamino)acetamide;

N-[4-(6-(3,4-dimethoxyphenyl)-3-oxo-2,3,4,5-tetrahydropyridazin-2-ylmethyl)phenyl]-2-(N,N-diisopropylamino)acetamide;

N-[4-(6-(3,4-dimethoxyphenyl)-3-oxo-2,3,4,5-tetrahydropyridazin-2-ylmethyl)phenyl]-2-thiomorpholinoacetamide;

N-[4-(6-(3,4-dimethoxyphenyl)-3-oxo-2,3,4,5-tetrahydropyridazin-2-ylmethyl)phenyl]-2-piperidinoacetamide;

N-[4-(6-(3,4-dimethoxyphenyl)-3-oxo-2,3,4,5-tetrahydropyridazin-2-ylmethyl)phenyl]-2-(4-hydroxypiperidino)acetamide;

N-[4-(6-(3,4-dimethoxyphenyl)-3-oxo-2,3,4,5-tetrahydropyridazin-2-ylmethyl)phenyl]-2-piperazinoacetamide;

N-[4-(6-(3,4-dimethoxyphenyl)-3-oxo-2,3,4,5-tetrahydropyridazin-2-ylmethyl)phenyl]-2-pyrrolidinoacetamide;

N-[4-(6-(3,4-dimethoxyphenyl)-3-oxo-2,3,4,5-tetrahydropyridazin-2-ylmethyl)phenyl]-3-(4-morpholinyl)propionamide;

N-[4-(6-(3,4-dimethoxyphenyl)-3-oxo-2,3,4,5-tetrahydropyridazin-2-ylmethyl)phenyl]-3-(N,N-diethylamino)propionamide;

N-[4-(6-(3,4-dimethoxyphenyl)-3-oxo-2,3,4,5-tetrahydropyridazin-2-ylmethyl)phenyl]-3-(N,N-diisopropylamino)propionamide;

N-[4-(6-(3,4-dimethoxyphenyl)-3-oxo-2,3,4,5-tetrahydropyridazin-2-ylmethyl)phenyl]-3-thiomorpholinopropionamide;

N-[4-(3,4-dimethoxyphenyl)-3-oxo-2,3,4,5-tetrahydropyridazin-2-ylmethyl)phenyl]-3-piperidinopropionamide;

N-[4-(6-(3,4-dimethoxyphenyl)-3-oxo-2,3,4,5-tetrahydropyridazin-2-ylmethyl)phenyl]-3-(4-hydroxypiperidino)propionamide;

N-[4-(6-(3,4-dimethoxyphenyl)-3-oxo-2,3,4,5-tetrahydropyridazin-2-ylmethyl)phenyl]-3-piperazinopropionamide;

N-[4-(6-(3,4-dimethoxyphenyl)-3-oxo-2,3,4,5-tetrahydropyridazin-2-ylmethyl)phenyl]-3-pyrrolidinopropionamide;

N-[4-(6-(3-propoxy-4-methoxyphenyl)-3-oxo-2,3,4,5-tetrahydropyridazin-2-ylmethyl)phenyl]-2-(4-morpholinyl)acetamide;

N-[4-(6-(3-propoxy-4-methoxyphenyl)-3-oxo-2,3,4,5-tetrahydropyridazin-2-ylmethyl)phenyl]-2-(N,N-diethylamino)acetamide;

N-[4-(6-(3-propoxy-4-methoxyphenyl)-3-oxo-2,3,4,5-tetrahydropyridazin-2-ylmethyl)phenyl]-2-(N,N-diisopropylamino)acetamide;

N-[4-(6-(3-propoxy-4-methoxyphenyl)-3-oxo-2,3,4,5-tetrahydropyridazin-2-ylmethyl)phenyl]-2-thiomorpholinoacetamide;

N-[4-(6-(3-propoxy-4-methoxyphenyl)-3-oxo-2,3,4,5-tetrahydropyridazin-2-ylmethyl)phenyl]-2-piperidinoacetamide;

N-[4-(6-(3-propoxy-4-methoxyphenyl)-3-oxo-2,3,4,5-tetrahydropyridazin-2-ylmethyl)phenyl]-2-(4-hydroxypiperidino)acetamide;

N-[4-(6-(3-propoxy-4-methoxyphenyl)-3-oxo-2,3,4,5-tetrahydropyridazin-2-ylmethyl)-2-piperazinoacetamide;

N-[4-(6-(3-propoxy-4-methoxyphenyl)-3-oxo-2,3,4,5-tetrahydropyridazin-2-ylmethyl)phenyl]-2-pyrrolidinoacetamide;

N-[4-(6-(3-isopropoxy-4-methoxyphenyl)-3-oxo-2,3,4,5-tetrahydropyridazin-2-ylmethyl)phenyl]-2-(4-morpholinyl)acetamide;

N-[4-(6-(3-isopropoxy-4-methoxyphenyl)-3-oxo-2,3,4,5-tetrahydropyridazin-2-ylmethyl)phenyl]-2-(N,N-diethylamino)acetamide;

N-[4-(6-(3-isopropoxy-4-methoxyphenyl)-3-oxo-2,3,4,5-tetrahydropyridazin-2-ylmethyl)phenyl]-2-(N,N-diisopropylamino)acetamide;

N-[4-(6-(3-isopropoxy-4-methoxyphenyl)-3-oxo-2,3,4,5-tetrahydropyridazin-2-ylmethyl)phenyl]-2-thiomorpholinoacetamide;

N-[4-(6-(3-isopropoxy-4-methoxyphenyl)-3-oxo-2,3,4,5-tetrahydropyridazin-2-ylmethyl)phenyl]-2-piperidinoacetamide;

N-[4-(6-(3-isopropoxy-4-methoxyphenyl)-3-oxo-2,3,4,5-tetrahydropyridazin-2-ylmethyl)phenyl]-2-(4-hydroxypiperidino)acetamide;

N-[4-(6-(3-isopropoxy-4-methoxyphenyl)-3-oxo-2,3,4,5-tetrahydropyridazin-2-ylmethyl)-2-piperazinoacetamide;

N-[4-(6-(3-isopropoxy-4-methoxyphenyl)-3-oxo-2,3,4,5-tetrahydropyridazin-2-ylmethyl)phenyl]-2-pyrrolidinoacetamide;

N-[4-(6-(3-cyclopentyloxy-4-methoxyphenyl)-3-oxo-2,3,4,5-tetrahydropyridazin-2-ylmethyl)phenyl]-2-(4-morpholinyl)acetamide;

N-[4-(6-(3-cyclopentyloxy-4-methoxyphenyl)-3-oxo-2,3,4,5-tetrahydropyridazin-2-ylmethyl)phenyl]-2-(N,N-diethylamino)acetamide;

N-[4-(6-(3-cyclopentyloxy-4-methoxyphenyl)-3-oxo-2,3,4,5-tetrahydropyridazin-2-ylmethyl)phenyl]-2-(N,N-diisopropylamino)acetamide;

N-[4-(6-(3-cyclopentyloxy-4-methoxyphenyl)-3-oxo-2,3,4,5-tetrahydropyridazin-2-ylmethyl)phenyl]-2-thiomorpholinoacetamide;

N-[4-(6-(3-cyclopentyloxy-4-methoxyphenyl)-3-oxo-2,3,4,5-tetrahydropyridazin-2-ylmethyl)phenyl]-2-piperidinoacetamide;

N-[4-(6-(3-cyclopentyloxy-4-methoxyphenyl)-3-oxo-2,3,4,5-tetrahydropyridazin-2-ylmethyl)phenyl]-2-(4-hydroxypiperidino)acetamide;

N-[4-(6-(3-cyclopentyloxy-4-methoxyphenyl)-3-oxo-2,3,4,5-tetrahydropyridazin-2-ylmethyl)-2-piperazinoacetamide;

N-[4-(6-(3-cyclopentyloxy-4-methoxyphenyl)-3-oxo-2,3,4,5-tetrahydropyridazin-2-ylmethyl)phenyl]-2-pyrrolidinoacetamide;

N-[4-(6-(3-ethoxy-4-methoxyphenyl)-3-oxo-2,3,4,5-tetrahydropyridazin-2-ylmethyl)phenyl]-3-(4-morpholinyl)propionamide;

N-[4-(6-(3-ethoxy-4-methoxyphenyl)-3-oxo-2,3,4,5-tetrahydropyridazin-2-ylmethyl)phenyl]-3-(N,N-diethylamino)propionamide;

N-[4-(6-(3-ethoxy-4-methoxyphenyl)-3-oxo-2,3,4,5-tetrahydropyridazin-2-ylmethyl)phenyl]-3-(N,N-diisopropylamino)propionamide;

N-[4-(6-(3-ethoxy-4-methoxyphenyl)-3-oxo-2,3,4,5-tetrahydropyridazin-2-ylmethyl)phenyl]-3-thiomorpholinoacetamide;

N-[4-(6-(3-ethoxy-4-methoxyphenyl)-3-oxo-2,3,4,5-tetrahydropyridazin-2-ylmethyl)phenyl]-3-piperidinopropionamide;

N-[4-(6-(3-ethoxy-4-methoxyphenyl)-3-oxo-2,3,4,5-tetrahydropyridazin-2-ylmethyl)phenyl]-3-(4-hydroxypiperidino)propionamide;

N-[4-(6-(3-ethoxy-4-methoxyphenyl)-3-oxo-2,3,4,5-tetrahydropyridazin-2-ylmethyl)phenyl]-3-piperazinopropionamide;

N-[4-(6-(3-ethoxy-4-methoxyphenyl)-3-oxo-2,3,4,5-tetrahydropyridazin-2-ylmethyl)phenyl]-3-pyrrolidinopropionamide;

N-[4-(6-(3-propoxy-4-methoxyphenyl)-3-oxo-2,3,4,5-tetrahydropyridazin-2-ylmethyl)phenyl]-3-(4-morpholinyl)propionamide;

N-[4-(6-(3-propoxy-4-methoxyphenyl)-3-oxo-2,3,4,5-tetrahydropyridazin-2-ylmethyl)phenyl]-3-(N,N-diethylamino)propionamide;

N-[4-(6-(3-propoxy-4-methoxyphenyl)-3-oxo-2,3,4,5-tetrahydropyridazin-2-ylmethyl)phenyl]-3-(N,N-diisopropylamino)propionamide;

N-[4-(6-(3-propoxy-4-methoxyphenyl)-3-oxo-2,3,4,5-tetrahydropyridazin-2-ylmethyl)phenyl]-3-thiomorpholinopropionamide;

N-[4-(6-(3-propoxy-4-methoxyphenyl)-3-oxo-2,3,4,5-tetrahydropyridazin-2-ylmethyl)phenyl]-3-piperidinopropionamide;

N-[4-(6-(3-propoxy-4-methoxyphenyl)-3-oxo-2,3,4,5-tetrahydropyridazin-2-ylmethyl)phenyl]-3-(4-hydroxypiperidino)propionamide;

N-[4-(6-(3-propoxy-4-methoxyphenyl)-3-oxo-2,3,4,5-tetrahydropyridazin-2-ylmethyl)phenyl]-3-piperazinopropionamide;

N-[4-(6-(3-propoxy-4-methoxyphenyl)-3-oxo-2,3,4,5-tetrahydropyridazin-2-ylmethyl)phenyl]-3-pyrrolidinopropionamide;

N-[4-(6-(3-isopropoxy-4-methoxyphenyl)-3-oxo-2,3,4,5-tetrahydropyridazin-2-ylmethyl)phenyl]-3-(4-morpholinyl)propionamide;

N-[4-(6-(3-isopropoxy-4-methoxyphenyl)-3-oxo-2,3,4,5-tetrahydropyridazin-2-ylmethyl)phenyl]-3-(N,N-diethylamino)propionamide;

N-[4-(6-(3-isopropoxy-4-methoxyphenyl)-3-oxo-2,3,4,5-tetrahydropyridazin-2-ylmethyl)phenyl]-3-(N,N-diisopropylamino)propionamide;

N-[4-(6-(3-isopropoxy-4-methoxyphenyl)-3-oxo-2,3,4,5-tetrahydropyridazin-2-ylmethyl)phenyl]-3-thiomorpholinopropionamide;

N-[4-(6-(3-isopropoxy-4-methoxyphenyl)-3-oxo-2,3,4,5-tetrahydropyridazin-2-ylmethyl)phenyl]-3-piperidinopropionamide;

N-[4-(6-(3-isopropoxy-4-methoxyphenyl)-3-oxo-2,3,4,5-tetrahydropyridazin-2-ylmethyl)phenyl]-3-(4-hydroxypiperidino)propionamide;

N-[4-(6-(3-isopropoxy-4-methoxyphenyl)-3-oxo-2,3,4,5-tetrahydropyridazin-2-ylmethyl)phenyl]-3-piperazinopropionamide;

N-[4-(6-(3-isopropoxy-4-methoxyphenyl)-3-oxo-2,3,4,5-tetrahydropyridazin-2-ylmethyl)phenyl]-3-pyrrolidinopropionamide;

N-[4-(6-(3-cyclopentyloxy-4-methoxyphenyl)-3-oxo-2,3,4,5-tetrahydropyridazin-2-ylmethyl)phenyl]-3-(4-morpholinyl)propionamide;

N-[4-(6-(3-cyclopentyloxy-4-methoxyphenyl)-3-oxo-2,3,4,5-tetrahydropyridazin-2-ylmethyl)phenyl]-3-(N,N-diethylamino)propionamide;

N-[4-(6-(3-cyclopentyloxy-4-methoxyphenyl)-3-oxo-2,3,4,5-tetrahydropyridazin-2-ylmethyl)phenyl]-3-(N,N-diisopropylamino)propionamide;

N-[4-(6-(3-cyclopentyloxy-4-methoxyphenyl)-3-oxo-2,3,4,5-tetrahydropyridazin-2-ylmethyl)phenyl]-3-thiomorpholinopropionamide;

N-[4-(6-(3-cyclopentyloxy-4-methoxyphenyl)-3-oxo-2,3,4,5-tetrahydropyridazin-2-ylmethyl)phenyl]-3-piperidinopropionamide;

N-[4-(6-(3-cyclopentyloxy-4-methoxyphenyl)-3-oxo-2,3,4,5-tetrahydropyridazin-2-ylmethyl)phenyl]-3-(4-hydroxypiperidino)propionamide;

N-[4-(6-(3-cyclopentyloxy-4-methoxyphenyl)-3-oxo-2,3,4,5-tetrahydropyridazin-2-ylmethyl)phenyl]-3-piperazinopropionamide;

N-[4-(6-(3-cyclopentyloxy-4-methoxyphenyl)-3-oxo-2,3,4,5-tetrahydropyridazin-2-ylmethyl)phenyl]-3-pyrrolidinopropionamide;

3-(N,N-diethylamino)propyl N-[4-(3,6-dihydro-5-(3,4-dimethoxyphenyl)-2-oxo-2H-1,3,4-thiadiazin-3-ylmethyl)phenyl]carbamate;

and/or physiologically acceptable salts thereof for the preparation of a medicament for the treatment of osteoporosis, tumours, tumour metastases, atherosclerosis, rheumatoid arthritis, multiple sclerosis, diabetes mellitus, ulcerative collitis and AIDS.

The invention relates, in particular, to the use of

a) compounds, disclosed in EP 0 738 715 A2, selected from the group consisting of

2-3-ethoxycarbonylaminobenzyl)-6-(3,4-dimethoxyphenyl)-2,3,4,5-tetrahydropyridazin-3-one;

2-3-methylsulfonamidobenzyl)-6-(3,4-dimethoxyphenyl)-2,3,4,5-tetrahydropyridazin-3-one;

2-3-acetamidobenzyl)-6-(3,4-dimethoxyphenyl)-2,3,4,5-tetrahydropyridazin-3-one;

2-4-trifluoroacetamidobenzyl)-6-(3,4-dimethoxyphenyl)-5-ethyl-2,3,4,5-tetrahydropyridazin-3-one;

2-4-ethoxycarbonylaminobenzyl)-6-(3,4-dimethoxyphenyl)-5-ethyl-2,3,4,5-tetrahydropyridazin-3-one;

2-(4-methoxycarbonylaminobenzyl)-6-(3,4-dimethoxyphenyl)-5-ethyl-2,3,4,5-tetrahydropyridazin-3-one;

2-(4-butyrylaminobenzyl)-6-(3,4-dimethoxyphenyl)-5-ethyl-2,3,4,5-tetrahydropyridazin-3-one;

b) compounds, disclosed in EP 0 723 962 B1, selected from the group consisting of

3-(4-nitrobenzyl)-5-(3,4-dimethoxyphenyl)-6-ethyl-3,6-dihydro-1,3,4-thiadiazin-2-one;

3-(4-aminobenzyl)-5-(3,4-dimethoxyphenyl)-6-ethyl-3,6-dihydro-1,3,4-thiadiazin-2-one;

3-(4-trifluoroacetamidobenzyl)-5-(3,4-dimethoxyphenyl)-6-ethyl-3,6-dihydro-1,3,4-thiadiazin-2-one;

3-(4-acetamidobenzyl)-5-(3,4-dimethoxyphenyl)-3,6-dihydro-1,3,4-thiadiazin-2-one;

3-(4-methoxybenzyl)-5-(3,4-dimethoxyphenyl)-6-ethyl-3,6-dihydro-1,3,4-thiadiazin-2-one;

3-(2,6-dichlorobenzyl)-5-(3,4-dimethoxyphenyl)-6-ethyl-3,6-dihydro-1,3,4-thiadiazin-2-one;

c) compounds, disclosed in EP 0 763 534 A1, selected from the group consisting of

3-4-picolinoylaminobenzyl)-5-(3,4-dimethoxyphenyl)-6-ethyl-3,6-dihydro-1,3,4-thiadiazin-2-one;

3-4-picolinoylaminobenzyl)-5-(3,4-dimethoxyphenyl)-3,6-dihydro-1,3,4-thiadiazin-2-one;

3-4-nicotinoylaminobenzyl)-5-(3,4-dimethoxyphenyl)-3,6-dihydro-1,3,4-thiadiazin-2-one;

3-4-isonicotinoylaminobenzyl)-5-(3,4-dimethoxyphenyl)-3,6-dihydro-1,3,4-thiadiazin-2-one;

3-4-nicotinoylaminobenzyl)-5-(3,4-dimethoxyphenyl)-6-ethyl-3,6-dihydro-1,3,4-oxadiazin-2-one;

2-4-nicotinoylaminobenzyl)-6-(3,4-dimethoxyphenyl)-5-ethyl-2,3,4,5-tetrahydropyridazin-3-one;

d) Compounds, disclosed in EP 0 539 306 B1 selected from the group consisting of

5-(3-methoxy-4-difluoromethoxyphenyl)-6-ethyl-3,6-dihydro-1,3,4-thiadiazin-2-one;

5-(3-methoxy-4-difluoromethoxyphenyl)-3,6-dihydro-1,3,4-thiadiazin-2-one;

e) compounds, disclosed in EP 0 618 201 A1, selected from the group consisting of

3-dimethylaminopropyl-5-(3,4-dimethoxyphenyl)-6-ethyl-3,6-dihydro-1,3,4-thiadiazinon-2-one;

3-dimethylaminopropyl-5-(3,4-dimethoxyphenyl)-3,6-dihydro-1,3,4-thiadiazinon-2-one;

3-dimethylaminoethyl-5-(3,4-dimethoxyphenyl)-6-ethyl-3,6-dihydro-1,3,4-thiadiazinon-2-one;

3-piperidinopropyl-5-(3-methoxy-4-difluoromethoxyphenyl)-6-ethyl-3,6-dihydro-1,3,4-thiadiazinon-2-one;

3-morpholinopropyl-5-(3-methoxy-4-difluoromethoxyphenyl)-6-ethyl-3,6-dihydro-1,3,4-thiadiazinon-2-one;

f) compounds, disclosed in DE 196 04 388 A1, selected from the group consisting of

3-(N,N-diethylamino)propyl N-[4-(3,6-dihydro-5-(3,4-dimethoxyphenyl)-2-oxo-2H-1,3,4-thiadiazin-3-ylmethyl)phenyl]carbamate;

3-(N,N-diethylamino)propyl N-[4-(3,6-dihydro-5-(3-ethoxy-4-methoxyphenyl)-2-oxo-2H-1,3,4-thiadiazin-3-ylmethyl)phenyl]carbamate;

3-(N,N-diethylamino)propyl N-[4-(6-(3,4-dimethoxyphenyl)-3-oxo-2,3,4,5-tetrahydropyridazin-2-ylmethyl)phenyl]carbamate;

3-(N,N-diethylamino)propyl N-[4-(6-(3-ethoxy-4-methoxyphenyl)-3-oxo-2,3,4,5-tetrahydropyridazin-2-ylmethyl)phenyl]carbamate;

N-[4-(6-(3-ethoxy-4-methoxyphenyl)-3-oxo-2,3,4,5-tetrahydropyridazin-2-ylmethyl)phenyl]-2-(4-methylpiperazino)acetamide;

N-[4-(6-(3-cyclopentyloxy-4-methoxyphenyl)-3-oxo-2,3,4,5-tetrahydropyridazin-2-ylmethyl)phenyl]-2-(4-methylpiperazino)acetamide;

N-[4-(3,6-dihydro-5-(3-ethoxy-4-methoxyphenyl)-2-oxo-2H-1,3,4-thiadiazin-3-ylmethyl)phenyl]-2-(4-methylpiperazino)acetamide;

and/or physiologically acceptable salts thereof for the preparation of a medicament for the treatment of osteoporosis, tumours, tumour metastases, atherosclerosis, rheumatoid arthritis, multiple sclerosis, diabetes mellitus, ulcerative collitis and AIDS.

1-5. (cancelled).

6. Method for the treatment of tumours and tumour metastases using compounds selected from the group consisting of
a) compounds of the formula I, disclosed in EP 0 738 715 A2, 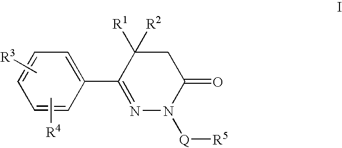
in which
R¹ and R² are each, independently of one another, H or A,
R³ and R⁴ are each, independently of one another, —OH, —OR¹⁰, —S—R¹⁰, —SO—R¹⁰, —SO₂—R¹⁰, Hal, methylenedioxy, —NO₂, —NH₂, —NHR¹⁰ or —NR¹⁰R¹¹,
R⁵ is a phenyl radical which is unsubstituted or monosubstituted or disubstituted by R⁶ and/or R⁷,
Q is absent or is alkylene having 1-6 carbon atoms,
R⁵ and R⁷ are each, independently of one another, —NH₂, —NR⁸R⁹, —NHR¹⁰, —NR¹⁰R¹¹, —NO₂, Hal, —CN, —OA, —COOH or —COOA,
R⁸ and R⁹ are each, independently of one another, H, acyl having 1-8 carbon atoms, which may be substituted by 1-5 F and/or Cl atoms, or are —COOA, —SO-A, —SO₂A, —CONH₂, —CONHA, —CONA₂, —CO—COOH, —CO—COOA, —CO—CONH₂, —CO—CONHA or —CO—CONA₂,
A is alkyl having from 1 to 6 carbon atoms, which may be substituted by 1-5 F and/or Cl atoms,
R¹⁰ and R¹¹ are each, independently of one another, A, cycloalkyl having 3-7 carbon atoms, methylenecycloalkyl having 4-8 carbon atoms or alkenyl having 2-8 carbon atoms,
and
Hal is F, Cl, Br or l,
b) compounds of the formula I, disclosed in EP 0 723 962 B1, 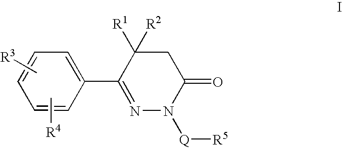
in which
R¹and R² are each, independently of one another, H or A,
R³ and R⁴ are each, independently of one another, —OH, —OR¹⁰, —S—R¹⁰, —SO—R¹⁰, —SO₂—R¹⁰, Hal, methylenedioxy, —NO₂, —NH₂, —NH¹⁰R¹¹,
R⁵ is a phenyl radical which is unsubstituted or monosubstituted or disubstituted by R⁶ and/or R⁷,
Q is absent or is alkylene having 1-6 carbon atoms,
R⁶ and R⁷ are each, independently of one another, —NH₂, —NR⁸R⁹, —NHR¹⁰, —NR¹⁰R¹¹, —NO₂, Hal, —CN, —OA, —COOH or —COOA,
R⁸ and R⁹ are each, independently of one another, H, acyl having 1-8 carbon atoms, which may be substituted by 1-5 F and/or Cl atoms, or are —COOA, —SO-A, —SO₂A, —CONH₂, —CONHA, —CONA₂, —CO—COOH, —CO—COOA, —CO—CONH₂, —CO—CONHA or —CO—CONA₂,
A is alkyl having from 1 to 6 carbon atoms, which may be substituted by 1-5 F and/or Cl atoms,
R¹⁰ and R¹¹ are each, independently of one another, A, cycloalkyl having 3-7 carbon atoms, methylenecycloalkyl having 4-8 carbon atoms or alkenyl having 2-8 carbon atoms,
and
Hal is F, Cl, Br or l,
c) compounds of the formula I, disclosed in EP 0 763 534 A1, 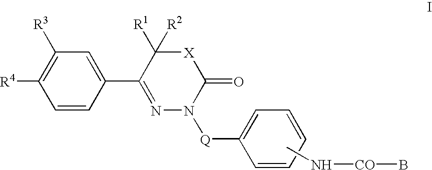
in which
B is an aromatic heterocyclic radical having from 1 to 4 N, O and/or S atoms, bonded via N or C, which may be unsubstituted or monosubstituted, disubstituted or trisubstituted by Hal, A and/or OA, and may also be fused to a benzene or pyridine ring,
Q is absent or is alkylene having 1-6 carbon atoms,
X is CH₂, S or O,
R¹ and R² are each, independently of one another, H or A,
R³ and R⁴ are each, independently of one another, —OH, —OR⁵, —S—R⁵, —SO—R⁵, —SO₂—R⁵, Hal, methylenedioxy, —NO₂, —NH₂, —NHR⁵ or —NR⁵R⁶,
R⁵ and R⁶ are each, independently of one another, A, cycloalkyl having 3-7 carbon atoms, methylenecycloalkyl having 4-8 carbon atoms or alkenyl having 2-8 carbon atoms,
A is alkyl having from 1 to 10 carbon atoms, which may be substituted by from 1 to 5 F and/or Cl atoms, and
Hal is F, Cl, Br or l,
d) compounds of the formula I, disclosed in EP 0 539 806 B1, 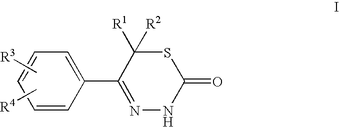
in which
R¹ and R² are each, independently of one another, H or A,
R³ is H, OA or O—CₘH₂ₘ₊₁₋ₙXₙ,
R⁴ is —O—CₘH₂ₘ₊₁₋ₙXₙ,
X is F or Cl,
A is alkyl having 1-6 carbon atoms,
m is 1, 2, 3, 4, 5 or 6, and
n is 1, 2, 3, 4, 5, 6, 7, 8, 9, 10, 11, 12 or 13,
e) compounds of the formula I, disclosed in EP 0 618 201 A1, 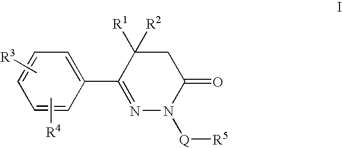
in which
R¹ and R² are each, independently of one another, H or A,
R³ and R⁴ are each, independently of one another, OH, OA, SA, SOA, SO₂A, Hal, methylenedioxy, cycloalkoxy having 3-7 carbon atoms or O—CₘH₂ₘ₊₁₋ₖFₖ,
R⁵ is —NR⁶R⁷ or 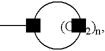
where one CH₂ group may also be replaced by oxygen,
R⁶ and R⁷ are each, independently of one another, H or A,
Q is alkylene having 1-6 carbon atoms,
A is alkyl having 1-6 carbon atoms,
Hal is F, Cl, Br or l,
m is 1, 2, 3, 4, 5 or 6,
n is 3, 4, 5 or 6,
k is 1, 2, 3, 4, 5, 6, 7, 8, 9, 10, 11, 12 or 13,
f) compounds of the formula I, disclosed in DE 196 04 388 A1, 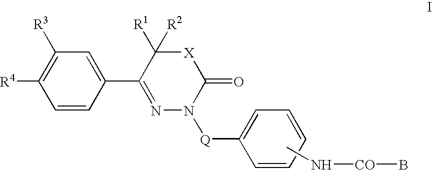
in which
R¹ and R² are each, independently of one another, H or A,
R³ and R⁴ are each, independently of one another, —OH, —OA, —S-A, —SO-A, —SO₂-A, Hal, methylenedioxy, —NO₂, —NH₂, —NHA or —NAA′,
A and A′ are each, independently of one another, alkyl having from 1 to 10 carbon atoms, which may be substituted by from 1 to 5 F and/or Cl atoms, or are cycloalkyl having 3-7 carbon atoms or methylenecycloalkyl having 4-8 carbon atoms,
B is —Y—R⁵ or —O—Y—R⁵,
Q is absent or is alkylene having 1-4 carbon atoms,
Y is absent or is alkylene having 1-10 carbon atoms,
X is CH₂ or S,
R⁵ is NH₂, NHA, NAA′ or a saturated 3-8-membered heterocyclic radical having at least one N atom which is unsubstituted or monosubstituted by A or OH and in which, in addition, further CH₂ groups may be replaced by NH, NA, S or O,
Hal is F, Cl, Br or l,
and physiologically acceptable salts thereof.

7. Method for the treatment of osteoporosis, atherosclerosis, rheumatoid arthritis, multiple sclerosis, diabetes mellitus, ulcerative colitis and AIDS using compounds selected from the group consisting of
a) compounds of the formula I, disclosed in EP 0 539 806 B1, 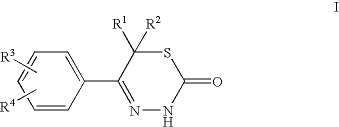
in which
R¹ and R² are each, independently of one another, H or A,
R³ is H, OA or O—CₘH₂ₘ₊₁₋ₙXₙ,
R⁴ is —O—CₘH₂ₘ₊₁₋ₙXₙ,
X is F or Cl,
A is alkyl having 1-6 carbon atoms,
m is 1, 2, 3, 4, 5 or 6, and
n is 1, 2, 3, 4, 5, 6, 7, 8, 9, 10, 11, 12 or 13,
b) compounds of the formula I, disclosed in EP 0 618 201 A1, 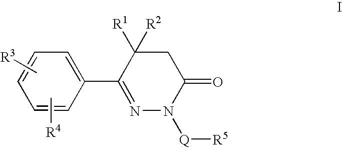
in which
R¹ and R² are each, independently of one another, H or A,
R³ and R⁴ are each, independently of one another, OH, OA, SA, SOA, SO₂A, Hal, methylenedioxy, cycloalkoxy having 3-7 carbon atoms or O—CₘH₂ₘ₊₁₋ₖFₖ,
R⁵ is —NR⁶R⁷ or 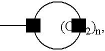
where one CH₂ group may also be replaced by oxygen,
R⁶ and R⁷ are each, independently of one another, H or A,
Q is alkylene having 1-6 carbon atoms,
A is alkyl having 1-6 carbon atoms,
Hal is F, Cl, Br or l,
m is 1, 2, 3, 4, 5 or 6,
n is 3, 4, 5 or 6,
k is 1, 2, 3, 4, 5, 6, 7, 8, 9, 10, 11, 12 or 13,
and physiologically acceptable salts thereof.

8. Method according to claim 6 using compounds selected from the group consisting of
a) compounds, disclosed in EP 0 738 715 A2, being
2-(4-ethoxycarbonylaminobenzyl)-6-(3,4-dimethoxyphenyl)-2,3,4,5-tetrahydropyridazin-3-one;
2-(3-methylsulfonamidobenzyl)-6-(3,4-dimethoxyphenyl)-2,3,4,5-tetrahydropyridazin-3-one;
2-(3-acetamidobenzyl)-6-(3,4-dimethoxyphenyl)-2,3,4,5-tetrahydropyridazin-3-one;
2-(4-trifluoroacetamidobenzyl)-6-(3,4-dimethoxyphenyl)-5-ethyl-2,3,4,5-tetrahydropyridazin-3-one;
2-(4-ethoxycarbonylaminobenzyl)-6-(3,4-dimethoxyphenyl)-5-ethyl-2,3,4,5-tetrahydropyridazin-3-one;
2-(4-methoxycarbonylaminobenzyl)-6-(3,4-dimethoxyphenyl)-5-ethyl-2,3,4,5-tetrahydropyridazin-3-one;
2-(4-butyrylaminobenzyl)-6-(3,4-dimethoxyphenyl)-5-ethyl-2,3,4,5-tetrahydropyridazin-3-one;
b) compounds, disclosed in EP 0 723 962 B1, being
3-(4-nitrobenzyl)-5-(3,4-dimethoxyphenyl)-6-ethyl-3,6-dihydro-1,3,4-thiadiazin-2-one;
3-(4-aminobenzyl)-5-(3,4-dimethoxyphenyl)-6-ethyl-3,6-dihydro-1,3,4-thiadiazin-2-one;
3-(4-trifluoroacetamidobenzyl)-5-(3,4-dimethoxyphenyl)-6-ethyl-3,6-dihydro-1,3,4-thiadiazin-2-one;
3-(4-acetamidobenzyl)-5-(3,4-dimethoxyphenyl)-3,6-dihydro-1,3,4-thiadiazin-2-one;
3-(4-methoxybenzyl)-5-(3,4-dimethoxyphenyl)-6-ethyl-3,6-dihydro-1,3,4-thiadiazin-2-one;
3-(2,6-dichlorobenzyl)-5-(3,4-dimethoxyphenyl)-6-ethyl-3,6-dihydro-1,3,4-thiadiazin-2-one;
c) compounds, disclosed in EP 0 763 534 A1, being
3-(4-picolinoylaminobenzyl)-5-(3,4-dimethoxyphenyl)-6-ethyl-3,6-dihydro-1,3,4-thiadiazin-2-one;
3-(4-picolinoylaminobenzyl)-5-(3,4-dimethoxyphenyl)-3,6-dihydro-1,3,4-thiadiazin-2-one;
3-(4-nicotinoylaminobenzyl)-5-(3,4-dimethoxyphenyl)-3,6-dihydro-1,3,4-thiadiazin-2-one;
3-(4-isonicotinoylaminobenzyl)-5-(3,4-dimethoxyphenyl)-3,6-dihydro-1,3,4-thiadiazin-2-one;
3-(4-nicotinoylaminobenzyl)-5-(3,4-dimethoxyphenyl)-6-ethyl-3,6-dihydro-1,3,4-oxadiazin-2-one;
2-(4-nicotinoylaminobenzyl)-6-(3,4-dimethoxyphenyl)-5-ethyl-2,3,4,5-tetrahydropyridazin-3-one;
d) compounds, disclosed in EP 0 539 806 B1, being
5-(3-methoxy-4-difluoromethoxyphenyl)-6-ethyl-3,6-dihydro-1,3,4-thiadiazin-2-one;
5-(3-methoxy-4-difluoromethoxyphenyl)-3,6-dihydro-1,3,4-thiadiazin-2-one;
e) compounds, disclosed in EP 0 618 201 A1, being
3-dimethylaminopropyl-5-(3,4-dimethoxyphenyl)-6-ethyl-3,6-dihydro-1,3,4-thiadiazinon-2-one;
3-dimethylaminopropyl-5-(3,4-dimethoxyphenyl)-3,6-dihydro-1,3,4-thiadiazinon-2-one;
3-dimethylaminoethyl-5-(3,4-dimethoxyphenyl)-6-ethyl-3,6-dihydro-1,3,4-thiadiazinon-2-one;
3-piperidinopropyl-5-(3-methoxy-4-difluoromethoxyphenyl)-6-ethyl-3,6-dihydro-1,3,4-thiadiazinon-2-one;
3-morpholinopropyl-5-(3-methoxy-4-difluoromethoxyphenyl)-6-ethyl-3,6-dihydro-1,3,4-thiadiazinon-2-one;
f) compounds, disclosed in DE 196 04 388 A1, being
3-(N,N-diethylamino)propyl N-[4-(3,6-dihydro-5-(3,4-dimethoxyphenyl)-2-oxo-2H-1,3,4-thiadiazin-3-ylmethyl)phenyl]carbamate;
3-(N,N-diethylamino)propyl N-[4-(3,6-dihydro-5-(3-ethoxy-4-methoxyphenyl)-2-oxo-2H-1,3,4-thiadiazin-3-ylmethyl)phenyl]carbamate;
3-(N,N-diethylamino)propyl N-[4-(6-(3,4-dimethoxyphenyl)-3-oxo-2,3,4,5-tetrahydropyridazin-2-ylmethyl)phenyl]carbamate;
3-(N,N-diethylamino)propyl N-[4-(6-(3-ethoxy-4-methoxyphenyl)-3-oxo-2,3,4,5-tetrahydropyridazin-2-ylmethyl)phenyl]carbamate;
N-[4-(6-(3-ethoxy-4-methoxyphenyl)-3-oxo-2,3,4,5-tetrahydropyridazin-2-ylmethyl)phenyl]-2-(4-methylpiperazino)acetamide;
N-[4-(6-(3-cyclopentyloxy-4-methoxyphenyl)-3-oxo-2,3,4,5-tetrahydropyridazin-2-ylmethyl)phenyl]-2-(4-methylpiperazino)acetamide;
N-[4-(3,6-dihydro-5-(3-ethoxy-4-methoxyphenyl)-2-oxo-2H-1,3,4-thiadiazin-3-ylmethyl)phenyl]-2-(4-methylpiperazino)acetamide;
and physiologically acceptable salts thereof.

9. Method according to claim 7 using compounds selected from the group consisting of
a) compounds, disclosed in EP 0 539 806 B1, being
5-(3-methoxy-4-difluoromethoxyphenyl)-6-ethyl-3,6-dihydro-1,3,4-thiadiazin-2-one;
5-(3-methoxy-4-difluoromethoxyphenyl)-3,6-dihydro-1,3,4-thiadiazin-2-one;
b) compounds, disclosed in EP 0 618 201 A1, being
3-dimethylaminopropyl-5-(3,4-dimethoxyphenyl)-6-ethyl-3,6-dihydro-1,3,4-thiadiazinon-2-one;
3-dimethylaminopropyl-5-(3,4-dimethoxyphenyl)-3,6-dihydro-1,3,4-thiadiazinon-2-one;
3-dimethylaminoethyl-5-(3,4-dimethoxyphenyl)-6-ethyl-3,6-dihydro-1,3,4-thiadiazinon-2-one;
3-piperidinopropyl-5-(3-methoxy-4-difluoromethoxyphenyl)-6-ethyl-3,6-dihydro-1,3,4-thiadiazinon-2-one;
3-morpholinopropyl-5-(3-methoxy-4-difluoromethoxyphenyl)-6-ethyl-3,6-dihydro-1,3,4-thiadiazinon-2-one;
and physiologically acceptable salts thereof.

10. Method according to claim 6 for the treatment of neoplastic damage.

11. Method according to claim 6 for the treatment of pre-cancerogenic damage.

12. Method according to claim 6 for regulating apoptosis in human cells.

13. Method according to claim 7 for the treatment of neoplastic damage.

14. Method according to claim 7 for the treatment of pre-cancerogenic damage.

15. Method according to claim 7 for regulating apoptosis in human cells.
